(19) United States
(12) Patent Application Publication
(10) Pub. No.: US 2012/0190659 A1
(43) Pub. Date: Jul. 26, 2012

(54) ANGIOGENESIS INHIBITORS

(75) Inventors: Elias James Corey, Cambridge, MA (US); Barbara Czakó, Cambridge, MA (US); László Kürti, Cambridge, MA (US); Akiko Mammoto, Brookline, MA (US); Donald E. Ingber, Boston, MA (US)

(73) Assignees: Children's Medical Center Corporation, Boston, MA (US); President and Fellows of Harvard College, Cambridge, MA (US)

(21) Appl. No.: 13/265,589

(22) PCT Filed: Apr. 16, 2010
(86) PCT No.: PCT/US10/01121

§ 371 Date: Apr. 11, 2012

Related U.S. Application Data
(60) Provisional application No. 61/214,327, filed on Apr. 22, 2009.

(51) Int. Cl. A61K 31/58; C07J 43/00; A61P 27/02; C07J 41/00

(52) U.S. Cl. 514/176; 540/106; 540/95; 540/97

(57) ABSTRACT

Compounds of Structural Formula I or pharmaceutically acceptable salts thereof, are effective inhibitors of angiogenesis:

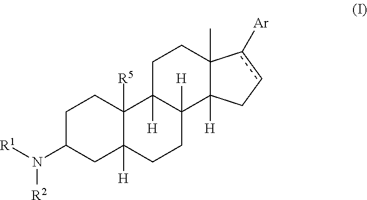

### PRIORITY INFORMATION

The present application claims priority under 35 U.S.C. §119(e) to U.S. provisional patent application, U.S. Ser. No. 61/214,327, filed Apr. 22, 2009, the entire contents of which are hereby incorporated by reference.

### GOVERNMENT SUPPORT

This invention was made with U.S. Government support under RO1 CA55833 and PO1 CA45548, awarded by the National Institutes of Health. The U.S. Government has certain rights in the invention.

### BACKGROUND OF THE INVENTION

Angiogenesis is a physiological process involving the sprouting of blood vessels from pre-existing blood vessels, characterized by endothelial cell proliferation and the proliferation and migration of tube forming cells. Angiogenesis is a normal process in growth and development, as well as in wound healing. Angiogenesis can be an aberrant and undesired process with detrimental consequences, such as the growth of solid tumors and metastasis, and hemangiomas. Aberrant angiogenesis can lead to certain pathological conditions such as death, blindness, and disfigurement.

Angiogenesis inhibitors can be used to treat various ‘angiogenesis-dependent’ diseases which result from enhanced or aberrant capillary growth, including age-related macular degeneration (hereinafter “ARMD”), diabetic retinopathy, psoriasis, atherosclerosis, arthritis and cancer, among others. ARMD is a degenerative condition of the macula (the central retina) which can cause vision loss in those 50 or older. Its prevalence increases with age. ARMD is caused by hardening of the arteries that nourish the retina. This deprives the sensitive retinal tissue of oxygen and nutrients the retina needs to function and thrive. As a result, the central vision deteriorates. About 10% of patients who suffer from macular degeneration have wet ARMD. This type occurs when new vessels form through angiogenesis to improve the blood supply to oxygen-deprived retinal tissue. However, the new vessels are very delicate and break easily, causing bleeding and damage to surrounding tissue. Angiogenesis inhibitors can be used to inhibit this damaging formation of new blood vessels.

Diabetic retinopathy can also be treated with the angiogenesis inhibitors. Diabetic retinopathy is a complication of diabetes and a leading cause of blindness. It occurs when diabetes damages the tiny blood vessels inside the retina. At this earliest stage of diabetic retinopathy, microaneurysms occur. These are small areas of balloon-like swelling in the retina's tiny blood vessels. As the disease progresses, some blood vessels that nourish the retina are blocked, depriving several areas of the retina with their blood supply. These areas of the retina send signals to the body to grow new blood vessels for nourishment. At this advanced stage, the signals sent by the retina for nourishment trigger the growth of new blood vessels. This condition is called proliferative retinopathy. These new blood vessels are abnormal and fragile. They grow along the retina and along the surface of the clear, vitreous gel that fills the inside of the eye.

By themselves, these blood vessels do not cause symptoms or vision loss. However, they have thin, fragile walls. If they leak blood, severe vision loss and even blindness can result. Fluid can leak into the center of the macula, the part of the eye where sharp, straight-ahead vision occurs. The fluid makes the macula swell, blurring vision. This condition is called macular edema. Angiogenesis inhibitors can be used to inhibit the formation of these abnormal and fragile blood vessels.

Psoriasis can also be treated with angiogenesis inhibitors. Psoriasis is a chronic skin disease occurring in approximately 3% of the population worldwide. It is characterized by excessive growth of the epidermal keratinocytes, inflammatory cell accumulation and excessive dermal angiogenesis. Alterations in the blood vessel formation of the skin are a prominent feature of psoriasis. Thus, angiogenesis inhibitors can be used to treat subject with this disease.

It is believed that angiogenesis inhibitors can be used to treat (therapeutically or prophylactically) atherosclerotic plaque formation, intimal hyperplasia and vascular restenosis. This is based on a number of studies which support the utility of inhibiting VEGF signaling to reduce restenosis: a) Shojima and Walsh, “The Role of Vascular Endothelial Growth Factor in Restenosis,” Circulation 110: 2283-2286 (2004); (b) Moulton et al., “Angiogenesis inhibitors endostatin or TNP-470 reduce intimal neovascularization and plaque growth in apolipoprotein E-deficient mice,” Circulation 99: 1726-1732 (1999); (c) Khurana et al., “Angiogenesis-dependent and independent phases of intimal hyperplasia,” Circulation 110: 2436-2443 (2004); (d) Ohtani et al., “Blockade of vascular endothelial growth factor suppresses experimental restenosis after intraluminal injury by inhibiting recruitment of monocyte lineage cells,” Circulation 110: 2444-2452 (2004).

Rheumatoid arthritis can also be treated with angiogenesis inhibitors. The expansion of the synovial lining of joints in rheumatoid arthritis (RA) and the subsequent invasion by the pannus of underlying cartilage and bone necessitate an increase in the vascular supply to the synovium, to cope with the increased requirement for oxygen and nutrients. The formation of new blood vessels is a key event in the formation and maintenance of the pannus in RA. This pannus is highly vascularized. Disruption of the formation of new blood vessels not only prevents delivery of nutrients to the inflammatory site, but could also lead to vessel regression and possibly reversal of disease (Angiogenesis inhibition suppresses collagen arthritis. Peacock D J, Banquerigo M L, Brahn E. J Exp Med. 1992 Apr. 1; 175(4):1135-8). The disclosed angiogenesis inhibitors can be used to disrupt the formation of new blood vessels in subjects with this disease.

Angiogenesis performs a critical role in the development of cancer. Solid tumors smaller than 1 to 2 cubic millimeters are not vascularized. Once they reach the critical volume of 2 cubic millimeters, oxygen and nutrients have difficulty diffusing to the cells in the center of the tumor, causing a state of cellular hypoxia that marks the onset of tumor angiogenesis. New blood vessel development is an important process in tumor progression. It favors the transition from hyperplasia to neoplasia, i.e., the passage from a state of cellular multiplication to a state of uncontrolled proliferation characteristic of tumor cells. Neovascularization also influences the dissemination of cancer cells throughout the entire body eventually leading to metastasis formation. The vascularization level of a solid tumor is thought to be an excellent indicator of its metastatic potential.

Angiogenesis inhibitors deprive malignant tissue of its oxygen and nutrient supply; in addition, it is unable to eliminate metabolic wastes. This in turn inhibits tumor progression and metastatic progression that accompanies most advanced cancers. Angiogenesis inhibitors can be used for these purposes as a treatment for cancers.

There is a need for new angiogenesis inhibitors for treating the aforementioned conditions and other angiogenesis-related diseases or disorders.

### SUMMARY OF THE INVENTION

New chemical moieties have been discovered that inhibit capillary cell growth, migration, and capillary tube formation in vitro, which have been found to be indicators of anti-angiogenic activity in animals and humans. In addition, they have been shown to directly inhibit angiogenesis in vivo in the living retina. These compounds are small, easily synthesized, and do not appear to exhibit significant toxicity in vitro or in vivo. They have been found to inhibit the angiogenic effects of VEGF. Multiple compounds exhibit an ability to inhibit cell sensitivity to many angiogenic factors. Further details are provided in the biological examples described herein.

One embodiment of the invention provides compounds represented by Structural Formula (I):

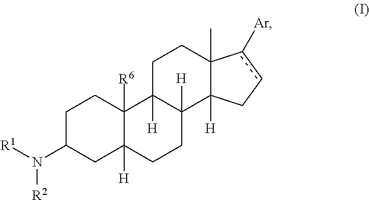

or a pharmaceutically acceptable salt thereof.

Ar is a heterocyclyl or heteroaryl, wherein each is monocyclic, bicyclic, or polycyclic, and wherein each is optionally substituted by one to three groups represented by R³, or aryl (e.g., phenyl) or cycloalkyl, wherein aryl and cycloalkyl represented by Ar are substituted with —[(CH₂)₀₋₆]—N(R⁴)₂ and optionally substituted by one or two groups represented by R³.

[symbol] is a single or double bond. In certain embodiments, [symbol] is a single bond. In certain embodiments, [symbol] is a double bond.

R¹ and R² are each independently hydrogen, a nitrogen-protecting group, (C₁-C₁₀)alkyl, (C₂-C₁₀)alkenyl, (C₂-C₁₀)alkynyl, aryl(C₀-C₆)alkyl, heteroaryl(C₀-C₆)alkyl, cycloalkyl(C₀-C₆)alkyl, heterocyclyl(C₀-C₆)alkyl, or heteroaryl(C₀-C₆)alkyl, each optionally substituted with one or more groups represented by R³. In certain embodiments, R¹ and R² are each independently hydrogen or (C₁-C₆)alkyl. In certain embodiments, R¹ and R² are each independently hydrogen or methyl. In certain embodiments, R¹ and R² are both methyl.

R¹ and R², along with the nitrogen to which they are attached, may form a monocyclic heterocyclyl moiety, optionally substituted by one or more groups selected from halogen, hydroxy, (C₁-C₆)alkyl, halo(C₁-C₆)alkyl, hydroxy(C₁-C₆)alkyl, (C₁-C₆)alkoxy, halo(C₁-C₆)alkoxy, —OC(O)R⁴, —C(O)R⁴, —C(O)OR⁴, —OC(═O)N(R⁴)₂, and oxo.

Each R³ is independently selected from halogen, nitro, cyano, hydroxy, (C₁-C₃)alkyl, halo(C₁-C₃)alkyl, hydroxy(C₁-C₃)alkyl, (C₁-C₃)alkoxy, halo(C₁-C₃)alkoxy, —(CH₂)_{y}—N(R⁴)₂, —(CH₂)_{y}—NR⁴CON(R⁴)₂, —(CH₂)_{y}—CON(R⁴)₂, —(CH₂)_{y}—N(R⁴)COR⁴, —(CH₂)_{y}—CO₂R⁴, —(CH₂)_{y}—OC(O)R⁴, —(CH₂)_{y}—SO₂N(R⁴)₂, —(CH₂)_{y}—SO₂R⁵, —(CH₂)_{y}—NR⁴CO₂R⁴, —(CH₂)_{y}—NR⁴SO₂R⁵, and —(CH₂)_{y}—OC(═O)N(R⁴)₂.

Each R⁴ is independently selected from hydrogen and (C₁-C₆)alkyl optionally substituted with halogen, hydroxyl, or (C₁-C₆)alkoxy.

Each R⁵ is independently selected from hydrogen, (C₁-C₆)alkyl and (C₁-C₆)alkoxy, wherein the alkyl or alkoxy is optionally substituted with halogen, hydroxyl, or (C₁-C₆)alkoxy;

R⁶ is hydrogen or C₁₋₆alkyl. Preferably, R⁶ is hydrogen or methyl. In certain embodiments, R⁶ is hydrogen. In other embodiments, R⁶ is methyl.

y is 0, 1, 2 or 3. In certain embodiments, the compounds of the invention inhibit angiogenesis and may be useful in the treatment of disease associated with aberrant or undesired angiogenesis.

In certain embodiments, the stereochemistry of the compound of Structural Formula (I) is as shown in Structural Formula (I′):

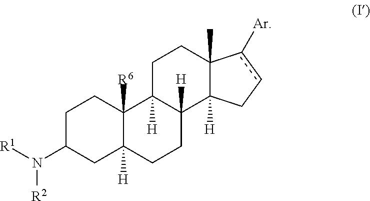

Another embodiment of the invention is method of inhibiting angiogenesis in a mammalian subject in need thereof, comprising administering to the subject an effective amount of an angiogenesis inhibitor disclosed herein.

Another embodiment of the invention is a pharmaceutical composition comprising an angiogenesis inhibitor disclosed herein and a pharmaceutically acceptable carrier or diluent.

Another embodiment of the invention is an angiogenesis inhibitor disclosed herein for use in medicinal therapy.

Another embodiment of the invention is the use of an angiogenesis inhibitor disclosed herein for the manufacture of a medicament for inhibiting angiogenesis in a mammalian subject in need of such treatment.

Another embodiment of the invention is an angiogenesis inhibitor disclosed herein for inhibiting angiogenesis in a mammalian subject in need of such treatment.

Another embodiment of the invention is a method of treating an angiogenesis-related disease or disorder in a mammalian subject, comprising administering to the subject an effective amount of an angiogenesis inhibitor disclosed herein.

All references cited herein, including patents, published patent applications, and publications, are incorporated by reference in their entirety.

### BRIEF DESCRIPTION OF THE DRAWINGS

FIG. 1 is a picture of a culture of the human umbilical vascular endothelial (HUVE) cells in 5% FBS/EGM2 media containing EJC-1, EJC-2, and EJC-3 at concentrations of 50 nM, 200 nM, 1000 nM, and 2000 nM. No morphological change is seen on the cells over the 24 hour culture period.

FIG. 2 is a picture of a culture of the human umbilical vascular endothelial (HUVE) cells in 5% FBS/EGM2 media containing EJC-4, EJC-5, and EJC-6 at concentrations of 50 nM, 200 nM, 1000 nM, and 2000 nM. No morphological change is seen on the cells over the 24 hour culture period.

FIG. 3A is a graph showing EJC-1 to EJC-2 inhibit 5-bromo-2′-deoxyuridine (BrdU) incorporation at 1 μM. A decrease in BrdU incorporation indicates that EJC-1 and EJC-2 inhibit the growth of HUVE cells.

FIG. 3B is a graph showing the effect of EJC-1 and EJC-2 on BrdU incorporation at 0, 200, 1000, and 2000 nM. A decrease in BrdU incorporation indicates that EJC-1 and EJC-2 inhibit the growth of HUVE cells.

FIG. 4 is a bar graph that shows the effects of EJC-10 on the growth of HUVE cells in the absence of growth factors (GF) or in the presence of VEGF, bFGF, and PDGF. The IC₅₀ of EJC-10 was 57.6 nM in a media absent an exogenous growth factor. The IC₅₀ of EJC-10 was 16.7, 64.07, and 79.48 nM in the presence of VEGF, bFGF, and PDGF, respectively.

FIG. 5 is a bar graph that shows the effects of EJC-14 on the growth of HUVE cells in the absence of GF or in the presence of VEGF, bFGF, and PDGF. EJC-14 inhibited HUVE cell growth induced by VEGF but not by bFGF and PDGF.

FIG. 6 is a bar graph that shows the effects of EJC-16 to EJC-20 on the growth of HUVE cells in the absence of GF or in the presence of VEGF. EJC-16 to EJC-20 inhibit cell growth induced by VEGF by half at 1000 nM.

FIG. 7A is a bar graph that shows the effects of EJC-1 to EJC-6 on the migration of HUVE cells in the presence of 1 μM of the test compound and 20 ng/ml of VEGF using transwell migration assay. EJC-1 and EJC-2 inhibit HUVE cell migration at 1 μM.

FIG. 7B is a bar graph that shows the effects of EJC-1 and EJC-2 on the migration of HUVE cells in the presence of 200, 1000, and 2000 nM of the test compound and 20 ng/ml of VEGF using transwell migration assay. EJC-1 inhibit HUVE cell migration at 2000 nM. EJC-2 inhibit HUVE migration at 1000 nM.

FIG. 8 is a bar graph that shows the effects of EJC-7 to EJC-10 on the migration of HUVE cells in the presence of 50 nM of the test compound and 20 ng/ml of VEGF using transwell migration assay. EJC-8 and EJC-10 inhibit HUVE cell migration at 50 nM.

FIG. 9 is a plot used for calculating the cell migration inhibition IC₅₀ of 70.7 nM for EJC-10.

FIG. 10 is a bar graph that shows the effects of EJC-11 to EJC-15 on the migration of HUVE cells in the presence of 50 nM of the test compound and 20 ng/ml of VEGF using transwell migration assay. EJC-12 and EJC-14 inhibit HUVE cell migration at 50 nM.

FIG. 11 is a bar graph that shows the effects of EJC-16 to EJC-20 on the migration of HUVE cells in the presence of either 50 or 200 nM of the test compound and 20 ng/ml of VEGF using transwell migration assay. EJC-16 to EJC-20 inhibit HUVE cell migration at 200 nM.

FIG. 12 is a bar graph that shows the effects of EJC-7 to EJC-12 on mean tube length of the tube formation by HUVE cells in vitro. EJC-9 and EJC-10 at 50 nM inhibit the tube formation when cultured with VEGF in the basal EBM2 medium.

FIG. 13 is a bar graph showing that injection of a single dose of 500 pmol of EJC-10 in the eye of a newborn mouse at postnatal day 6 (p6) decreases the density of the vessel formed in the retina of the mouse as compared to the retina of the control mouse.

### DETAILED DESCRIPTION OF THE INVENTION

The invention is directed to novel compounds which may be angiogenesis inhibitors and their use in treating disorders for which a beneficial clinical effect can be achieved by inhibiting angiogenesis. Macular degeneration is an example of a disorder of this type. Cancer is another example.

A compound of the present invention is represented by Structural Formula (I) or a pharmaceutically acceptable salt thereof. Alternatively, the compound is represented by Structural Formula (II) or (III):

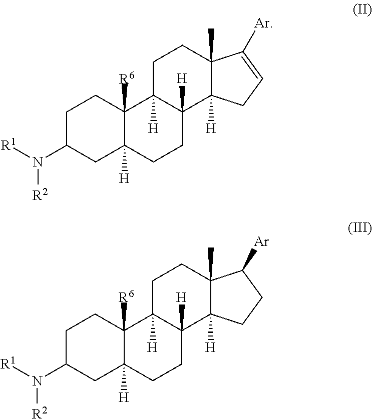

or a pharmaceutically acceptable salt thereof. A first set of possibilities for the variables in Structural Formula (II) or (III) is as defined above for Structural Formula (I). Alternatively, a second set of possibilities for the variables for Structural Formulae (II) and (III) are defined as follows:

R¹ and R² are each independently hydrogen; or (C₁-C₆)alkyl, optionally substituted with one or more groups represented by R³; or

R¹ and R², along with the nitrogen to which they are attached, form a monocyclic heterocyclyl, optionally substituted by one or more groups selected from the group consisting of halogen, hydroxy, (C₁-C₆)alkyl, halo(C₁-C₆)alkyl, hydroxy(C₁-C₆)alkyl, (C₁-C₆)alkoxy, halo(C₁-C₆)alkoxy, —OC(O)R⁴, —C(O)R⁴, —C(O)OR⁴, —OC(═O)N(R⁴)₂, and oxo; and

Ar is selected from the group consisting of pyrrolidinyl, pyrrolyl, pyrazolyl, triazolyl, tetrazolyl, imidazolyl, piperidinyl, piperazinyl, pyridinyl, pyridazinyl, pyrimidinyl, pyrazinyl, triazinyl, indolyl, isoindolyl, indolinyl, benzoimidazolyl, purinyl, benzotriazolyl, quinolinyl, tetrahydroquinolinyl, isoquinolinyl, or tetrahydroisoquinolinyl, wherein the heterocyclyl or heteroaryl is optionally substituted by one to three groups represented by R³. Preferably, Ar is pyridinyl, quinolinyl, tetrahydroquinolinyl, isoquinolinyl or tetrahydroisoquinolinyl, each optionally substituted by one to three groups represented by R³, or wherein Ar is -Ph-(CH₂)ₓN(R⁴)₂; Ph is phenyl which in addition to (CH₂)ₓN(R⁴)₂ is optionally substituted with one or two groups represented by R³; and x is an integer from 0 to 3; and

R³-R⁶ and y are as defined above for structural Formula (I).

A third set of values for the variables in Structural Formulas (II) and (III) are defined as follows:

R¹ and R² are independently hydrogen or (C₁-C₆)alkyl, hydroxy(C₁-C₆)alkyl, or (C₁-C₆)alkoxy(C₁-C₆)alkyl; or

R¹ and R² taken together with the nitrogen atom to which they are attached are a (C₃-C₇)heterocyclyl, optionally substituted with (C₁-C₆)alkyl, oxo, hydroxyl, or —C(O)(C₁-C₆)alkyl; and

each R³ is independently (C₁-C₆)alkyl, hydroxy, (C₁-C₆)alkoxy, halo(C₁-C₆)alkyl, halo(C₁-C₆)alkoxy, or hydroxy(C₁-C₆)alkyl; and

R⁶ and Ar are as defined above in the second set of values for Structural Formulae (II) and (III).

For the three sets of possibilities for Structural Formulae (II) and (III), R¹ and R² are preferably methyl.

In certain embodiments, R⁶ is hydrogen. In certain embodiments, R⁶ is methyl. In certain embodiments, R⁶ is ethyl. In certain embodiments, R⁶ is propyl. In certain embodiments, R⁶ is butyl.

In another alternative, the compound of the invention is represented by a structural formula selected from Structural Formulae (IV)-(XXIV):

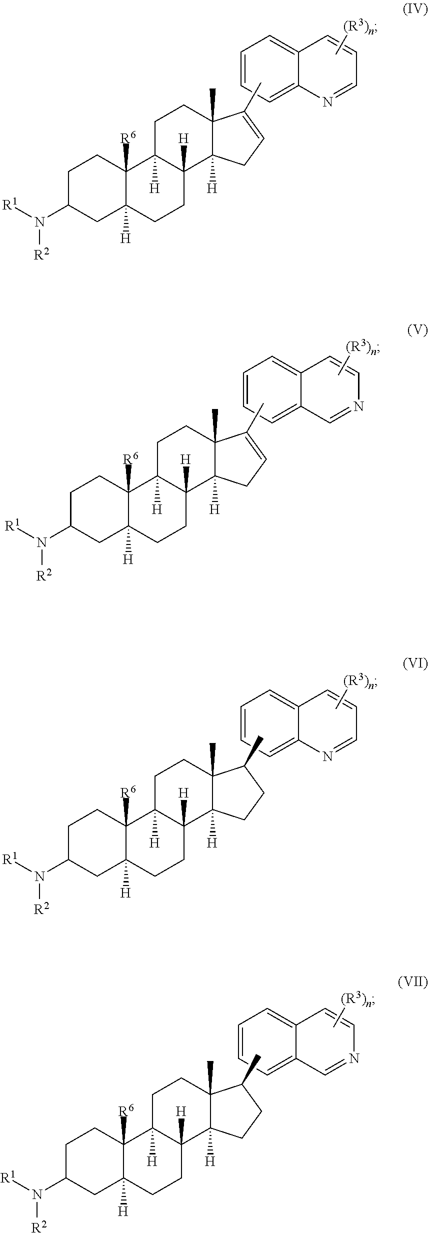

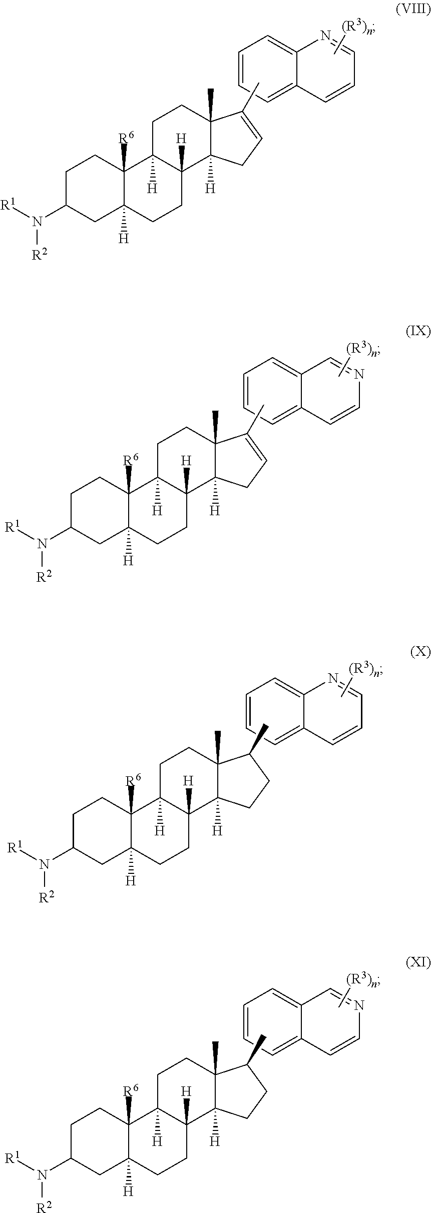

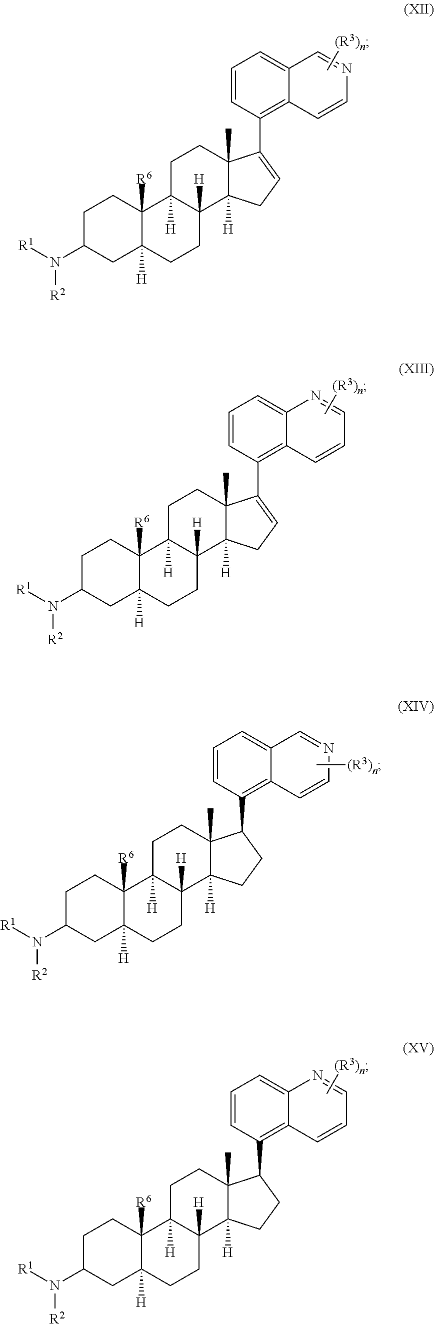

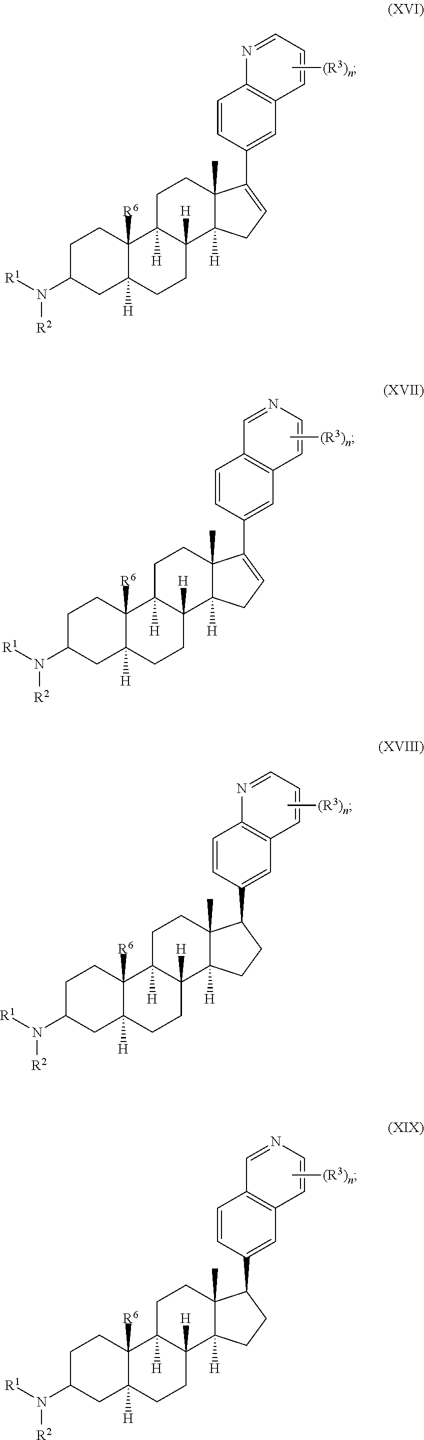

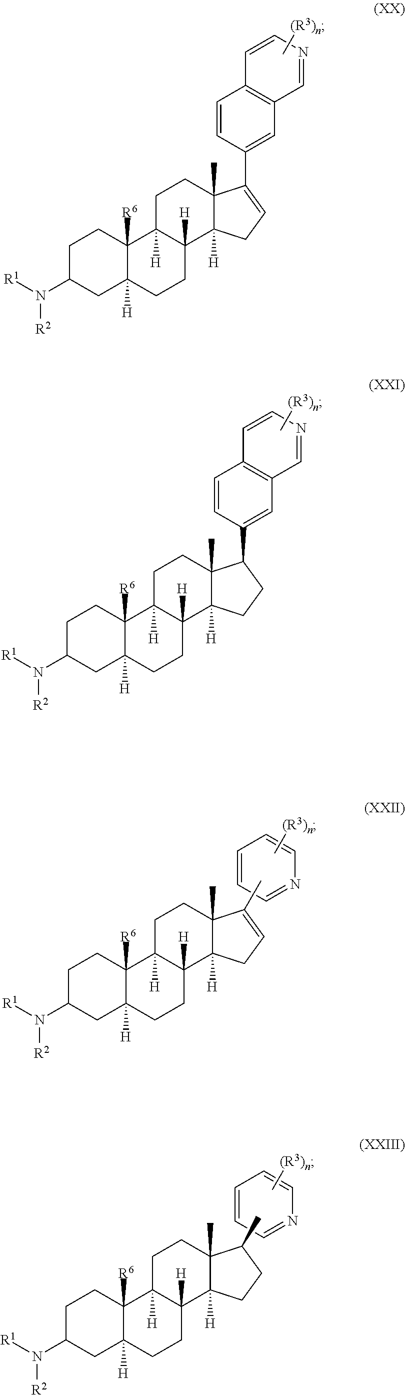

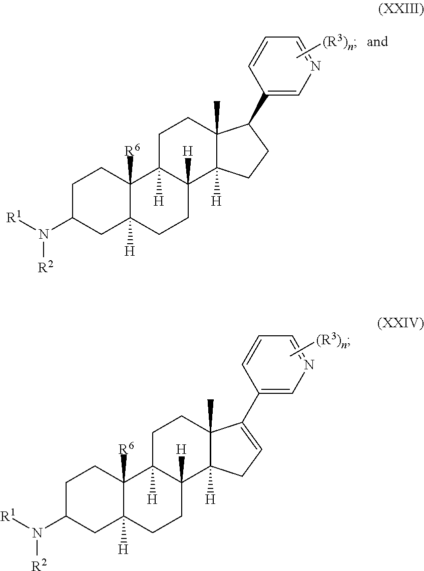

or a pharmaceutically acceptable salt thereof “n” in Structural Formulae (IV)-(XXIV) is an integer from 0-3, and the remainder of the variables in Structural Formulae (IV)-(XXIV) are as described in any one of the three sets of possibilities for the variables in Structural Formulae (II) and (III).

A second set of possibilities for the variables in Structural Formulae (IV)-(XXIV) are as follows:

R¹ and R² are independently hydrogen, (C₁-C₃)alkyl, hydroxy(C₁-C₃)alkyl, or (C₁-C₃)alkoxy(C₁-C₃)alkyl; or

R¹ and R² taken together with the nitrogen atom to which they are attached are a (C₃-C₇)heterocyclyl, optionally substituted with (C₁-C₃)alkyl, oxo, hydroxyl, or —C(O)(C₁-C₃)alkyl;

each R³ is independently (C₁-C₃)alkyl, hydroxy, (C₁-C₃)alkoxy, halo(C₁-C₃)alkyl, halo(C₁-C₃)alkoxy or hydroxy(C₁-C₃)alkyl;

R⁶ is hydrogen or methyl; and

n is an integer from 0 to 3.

A third set of values for the variables in Structural Formulas (IV)-(XXIV) are as follows:

R¹ and R² are methyl;

each R³ is independently (C₁-C₃)alkyl, hydroxy, (C₁-C₃)alkoxy, halo(C₁-C₃)alkyl, halo(C₁-C₃)alkoxy or hydroxy(C₁-C₃)alkyl;

R⁶ is hydrogen or methyl; and

n is 0 or 1.

In another alternative, the invention provides a compound represented by a structural formula selected from Structural Formulae (XXV)-(XXVI):

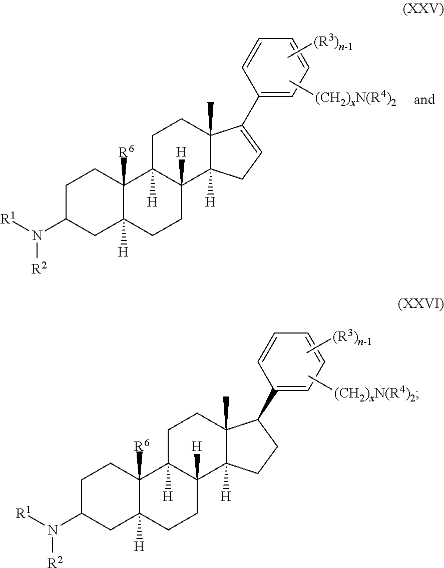

or a pharmaceutically acceptable salt thereof, wherein —(CH₂)ₓN(R⁴)₂ is meta or para to the ring carbon atom that is bonded to the cyclopentane or cyclopentene ring; n is 1, 2 or 3; and values for R¹⁻⁴, R⁶, and x are as described in any one of the three sets of values for the variables in Structural Formulas (II) and (III). Another set of values for the variables in Structural Formulas (XXV) and (XXVI) is defined as follows:

R¹ and R² are independently hydrogen or (C₁-C₃)alkyl, (C₁-C₃)hydroxyalkyl, or (C₁-C₃)alkoxy(C₁-C₃)alkyl; or

R¹ and R² taken together with the nitrogen atom to which they are attached are a (C₃-C₇)heterocyclyl, optionally substituted with (C₁-C₃)alkyl, oxo, hydroxyl, or —C(O)(C₁-C₃)alkyl;

each R³ is independently (C₁-C₃)alkyl, hydroxy, (C₁-C₃)alkoxy, (C₁-C₃)haloalkyl, (C₁-C₃)haloalkoxy, or (C₁-C₃)hydroxyalkyl;

each R⁴ is independently selected from hydrogen and (C₁-C₅)alkyl, optionally substituted with halogen, hydroxyl, or (C₁-C₃)alkoxy;

R⁶ is hydrogen or methyl;

(CH₂)_{X}N(R⁴)₂ is meta or para to the ring carbon atom that is bonded to the cyclopentane or cyclopentene ring;

n is 1, 2 or 3; and

x is an integer from 0 to 3.

A second set of values for the variables in Structural Formulas (XXV) and (XXVI) is defined as follows:

R¹ and R² are each methyl;

each R³ is independently (C₁-C₃)alkyl, hydroxy, (C₁-C₃)alkoxy, halo(C₁-C₃)alkyl, halo(C₁-C₃)alkoxy, or hydroxy(C₁-C₃)alkyl;

each R⁴ is independently hydrogen or (C₁-C₃)alkyl;

R⁶ is hydrogen or methyl;

(CH₂)_{X}N(R⁴)₂ is meta or para to the ring carbon atom that is bonded to the cyclopentane or cyclopentene ring;

n is 1 or 2; and

x is 1.

Specific examples of angiogenesis inhibitors of the invention are shown below.

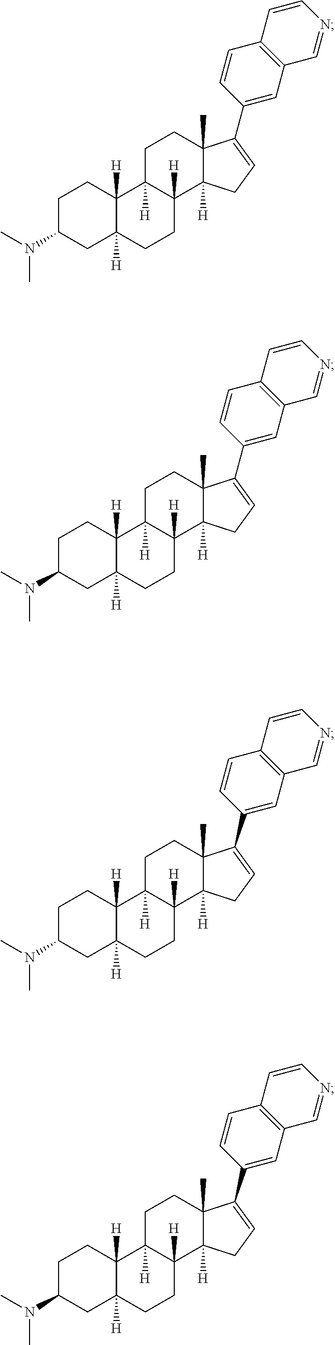

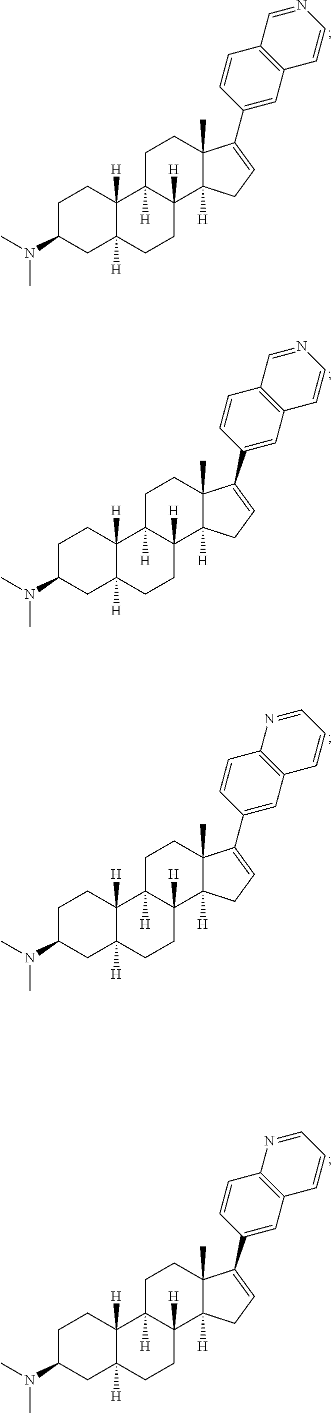

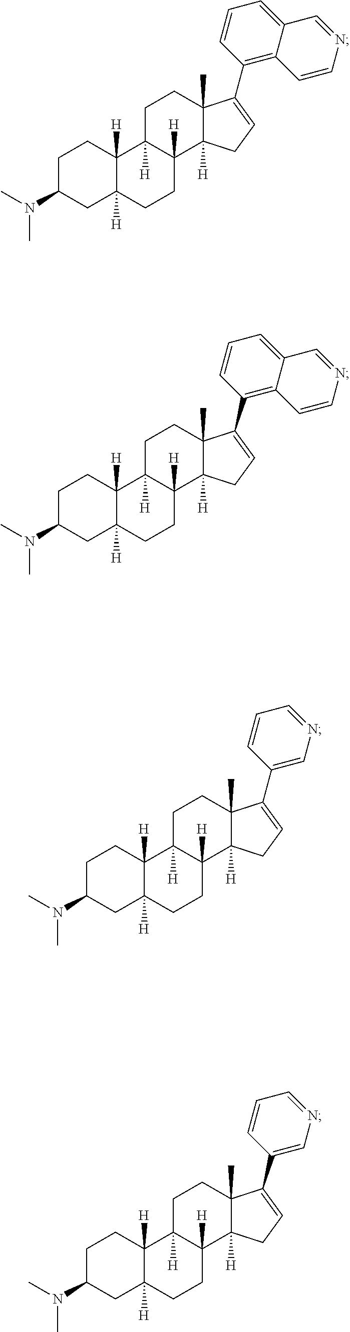

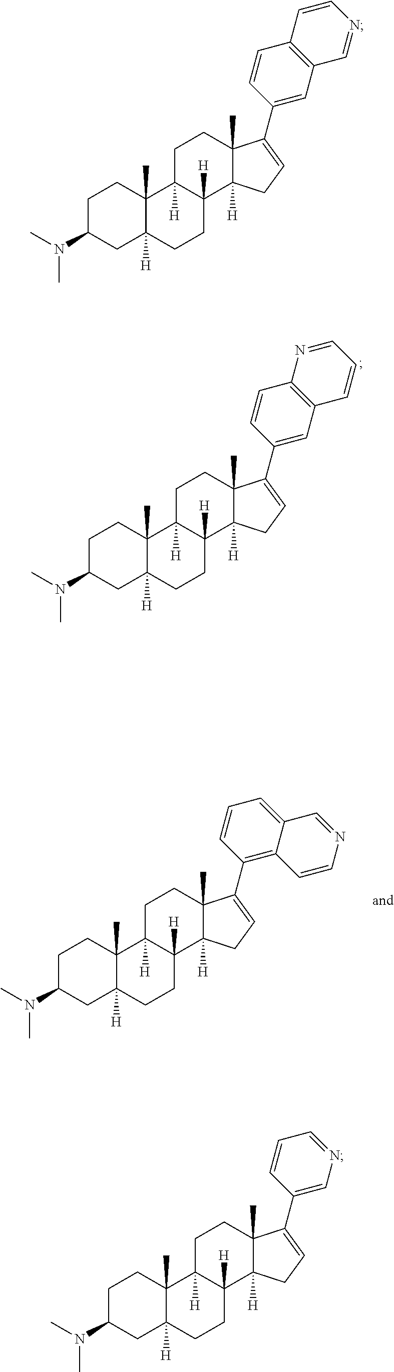

or a pharmaceutically acceptable salt thereof.

Alternative examples of angiogenesis inhibitors of the invention include compounds in which the 3β-dimethylamino group is replaced by a 3β-pyrrolidino or 3β-morpholino group:

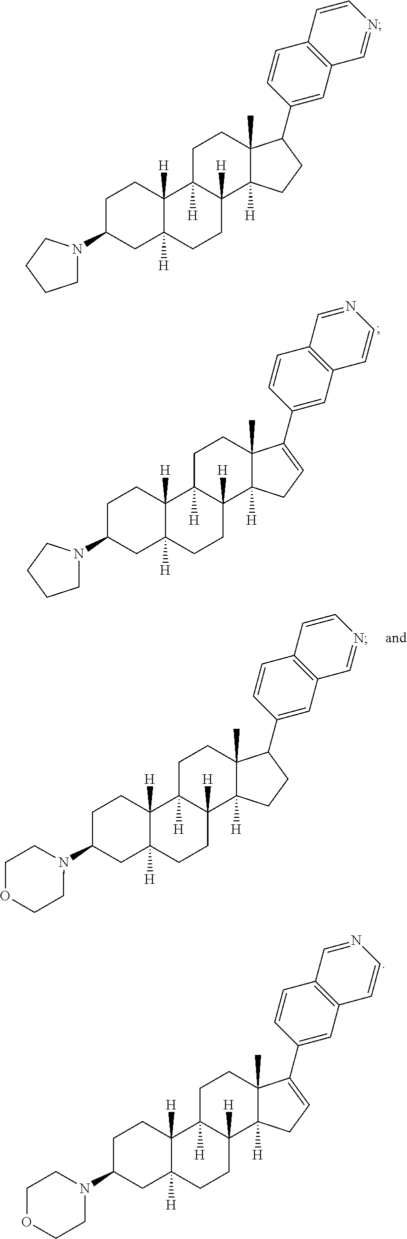

or a pharmaceutically acceptable salt thereof.

The invention also provides compounds of the Structural Formula:

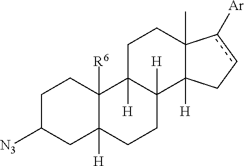

wherein R⁶ and Ar are as defined herein. The azido compound may be an angiogenesis inhibitor itself or an intermediate to the synthesis of a compounds described herein.

The term “angiogenesis,” as used herein, refers to the sprouting of blood vessels from pre-existing blood vessels, characterized by endothelial cell proliferation and the proliferation and migration of tube forming cells. Angiogenesis can be triggered by certain pathological conditions, such as the growth of solid tumors and metastasis. Angiogenesis can be a good and necessary process, for example, in wound healing, or it can be an aberrant and undesired process with detrimental consequences, such as the growth of solid tumors and metastasis, and hemangiomas. Aberrant angiogenesis can lead to certain pathological conditions such as death, blindness, and disfigurement.

As used herein, the term “angiogenic-related disease or disorder” refers to diseases or disorders that are the direct result of aberrant blood vessel proliferation (e.g., diabetic retinopathy and hemangiomas) or undesired or pathological blood vessel proliferation (e.g., in the case cancer and tumor growth). The term also refer to diseases or disorders whose pathological progression is dependent on a good blood supply and thus blood vessel proliferation. Examples include abnormal vascular proliferation, ascites formation, psoriasis, age-related macular degeneration, thyroid hyperplasia, preeclampsia, rheumatoid arthritis and osteo-arthritis, Alzheimer's disease, obesity, pleural effusion, atherosclerosis, endometriosis, diabetic/other retinopathies, ocular neovascularizations such as neovascular glauocoma and corneal neovascularization. The term “angiogenesis-related disease or disorder” and “angiogenic disease or disorder” are used interchangeably herein.

A subject is in need of treatment to inhibit angiogenesis when the subject has a disease or condition for which a beneficial therapeutic or prophylactic effect can be achieved by inhibiting angiogenesis either systemically or locally in the subject. Examples of subjects of this type are those who are being treated to inhibit angiogenesis for the purpose of treating macular degeneration (e.g., wet macular degeneration), diabetic retinopathy, rheumatoid arthritis, psoriasis, restenosis or cancer. The invention provides a method of treating an angiogenesis-related disease or disorder in a mammalian subject, comprising administering to the subject an effective amount an angiogenesis inhibitor disclosed herein. Examples of cancers which can be treated by inhibiting angiogenesis include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Because increased bone marrow (BM) angiogenesis has been demonstrated in hematologic malignancies, it is believed that the disclosed angiogenesis inhibitors will be effective in treating multiple myeloma, chronic myeloid leukemia, acute myeloid or lymphocytic leukemia, chronic lymphocytic leukemia (CLL) as well as myelodysplastic syndromes. See Vacca et al. “Bone Marrow Angiogenesis and Progression in Multiple Myeloma Br J Haematol, 87:503-8 (1994); Aguayo et al., “Angiogenesis in Acute and Chronic Leukaemia and Myelodysplastic Syndromes. Blood 96:2240-5 (2000); Hussong et al., “Evidence of Increased Angiogenesis in Patients with Acute Myeloid Leukaemia, Blood 95:309-13 (2000); Perez-Atayde et al., “Spectrum of Tumor Angiogenesis in the Bone Marrow of Children with Acute Lymphoblastic Leukemia, Am J Pathol 150:815-21 (1997); Molica et al., “Prognostic Value of Enhanced Bone Marrow Angiogenesis in Early B-Cell Chronic Lymphocytic Leukemia, Blood 100:3344-51 (2002); and Cheson et al., “National Institute-Sponsored Working Group Guidelines for Chronic Lymphocytic Leukemia: Revised Guidelines for Diagnosis and Treatment, Blood 87:4990-7 (1996).

Other diseases and conditions which can be treated with the disclosed angiogenesis inhibitors include corneal graft rejection; neovascular glaucoma; retrolental fibroplasias; epidemic keratoconjunctivitis; Vitamin A deficiency; contact lens overwear; atopic keratitis; superior limbic keratitis; pterygium keratitis sicca; sjogrens; acne; rosacea; wartsphylectenulosis; lipid degeneration; chemical burns; Terrien's marginal degeneration; mariginal keratolysis; rheumatoid arthritis; polyarteritis; Wegener's sarcoidosis; scleritis; Stevens-Johnson disease; pemphigoid; radial keratotomy; corneal graph rejection; sickle cell anemia; sarcoid; pseudoxanthoma elasticum; Paget's disease; vein occlusion; carotid obstructive disease; chronic uveitis/vitritis; Eales' disease; Behcet's disease; infections causing a retinitis or choroiditis; presumed ocular histoplasmosis; Best's disease; myopia; optic pits; Stargardt's disease; pars planitis; chronic retinal detachment; hyperviscosity syndromes; diseases associated with rubeosis (neovasculariation of the angle); osteoarthritis; ulcerative colitis; Crohn's disease; BartonellosisOsler-Weber-Rendu disease; hereditary hemorrhagic telangiectasia; pulmonary hemangiomatosis; pre-eclampsia; fibrosis of the liver and of the kidney; developmental abnormalities (organogenesis); skin disclolorations (e.g., hemangioma, nevus flammeus, or nevus simplex); hypertrophic scars, i.e., keloids; wound granulation; vascular adhesions; cat scratch disease (Rochele ninalia quintosa); keratoconjunctivitis; gingivitis; epulis; tonsillitis; obesity; laryngitis; tracheitis; bronchiolitis; pulmonary edema; neurodermitis; thyroiditis; thyroid enlargement; glomerulonephritis; gastritis; inflammatory bone and cartilage destruction; thromboembolic disease; Alzheimer's disease; obesity; endometriosis; and Buerger's disease.

“Treatment” or “treating” refers to both therapeutic and prophylactic treatment.

An “effective amount” is the quantity of an angiogenesis inhibitor in which a beneficial clinical outcome (prophylactic or therapeutic) is achieved when the compound is administered to a subject in need of treatment. For the treatment of rheumatoid arthritis or psoriasis, a “beneficial clinical outcome” includes a reduction in the severity of the symptoms associated with the disease (e.g., pain and inflammation), and/or a delay in the onset of the symptoms associated with the disease compared with the absence of the treatment. For the treatment of cancer, a beneficial clinical outcome includes a reduction in tumor mass, a reduction in the rate of tumor growth, a reduction in metastasis, a reduction in the severity of the symptoms associated with the cancer and/or an increase in the longevity of the subject compared with the absence of the treatment. For ocular diseases, a “beneficial clinical outcome” includes a reduction in the formation of abnormal blood vessels in the eye and the leakage and symptoms associated therewith, including loss of vision. The precise amount of angiogenesis inhibitor administered to a subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. Effective amounts of the disclosed compounds typically range between about 0.1 mg/kg body weight per day and about 1000 mg/kg body weight per day, and preferably between 1 mg/kg body weight per day and 100 mg/kg body weight per day.

The angiogenesis inhibitors described herein, and the pharmaceutically acceptable salts, thereof can be used in pharmaceutical preparations in combination with a pharmaceutically acceptable carrier or diluent. Suitable pharmaceutically acceptable carriers include inert solid fillers or diluents and sterile aqueous or organic solutions. The angiogenesis inhibitor will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

The angiogenesis inhibitors disclosed herein are suitable for oral administration because they are of low molecular weight and are water soluble. For oral administration, the angiogenesis inhibitor or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch, alginic acid; a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parenteral administration the disclosed angiogenesis, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms. Aqueous solutions with up to 20% hydroxypropyl ∃-cyclodextrin are commonly used.

In addition to the formulations described previously, the compounds may also be formulated as a long acting formulation, such as a depot preparation. Such long acting formulations may be administered by implantation, or, for example, subcutaneously by intramuscular injection. Depot formulations may be prepared from synthetic hydrogels such as those disclosed in U.S. Pat. Nos. 5,410,016; 6,177,095; and 6,632,457; the entire teachings of which are incorporated herein by reference. In certain applications, long acting formulations are implanted locally at the site of manifestation of the disease, for example, near, on or proximal to the affected organ or tissue.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., Rabinowitz, J D and Zaffaroni, A C, U.S. Pat. No. 6,803,031, assigned to Alexza Molecular Delivery Corporation, which is incorporated herein by reference.

For treating ocular diseases, such as macular degeneration, methods, of administration such that the angiogenesis inhibitor will contact an ocular cell are preferred. As such, the angiogenesis inhibitor can be appropriately formulated and administered in the form of an injection, eye lotion, eye drops, ointment, implant and the like. The angiogenesis inhibitor can be applied, for example, systemically, topically, subconjunctivally, intraocularly, retrobulbarly, periocularly, subretinally, or suprachoroidally.

Topical formulations are well known to those of skill in the art. Such formulations are suitable in the context of the present invention for application to the skin. The use of patches, corneal shields (see, e.g., U.S. Pat. No. 5,185,152, which is incorporated herein by reference), and ophthalmic solutions (see, e.g., U.S. Pat. No. 5,710,182, which is incorporated herein by reference) and ointments, e.g., eye drops, is also within the skill in the art. The expression vector can also be administered non-invasively using a needleless injection device, such as the Biojector 2000 Needle-Free Injection Management System™ available from Bioject, Inc.

The angiogenesis inhibitor can be administered from a device that allows controlled or sustained release, such as an ocular sponge, meshwork, mechanical reservoir, or mechanical implant. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493; each of which is incorporated herein by reference), such as an implantable device, e.g., a mechanical reservoir, an intraocular device or an extraocular device with an intraocular conduit, or an implant or a device comprised of a polymeric composition are particularly useful for ocular administration of the angiogenesis inhibitor. The angiogenesis inhibitor can also be administered in the form of sustained-release formulations (see, e.g., U.S. Pat. No. 5,378,475; incorporated herein by reference) comprising, for example, gelatin, chondroitin sulfate, a polyphosphoester, such as bis-2-hydroxyethyl-terephthalate (BHET), or a polylactic-glycolic acid.

Alternatively, the angiogenesis inhibitor can be administered using invasive procedures, such as, for instance, intravitreal injection or subretinal injection optionally preceded by a vitrectomy. Subretinal injections can be administered to different compartments of the eye, i.e., the anterior chamber. While intraocular injection is preferred, injectable compositions can also be administered intramuscularly, intravenously, and intraperitoneally. Pharmaceutically acceptable carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986)).

Preferably disclosed angiogenesis inhibitors or pharmaceutical formulations containing these compounds are in unit dosage form for administration to a mammalian subject. As used herein the terms “subject” and “patient” may be used interchangeably, and means a mammal in need of treatment, e.g., companion animals (e.g., dogs, cats, and the like), farm animals (e.g., cows, pigs, horses, sheep, goats, and the like) and laboratory animals (e.g., rats, mice, guinea pigs, and the like). Typically, the subject is a human in need of treatment.

The unit dosage form can be any unit dosage form known in the art including, for example, a capsule, an IV bag, a tablet, or a vial. The quantity of the angiogenesis inhibitor in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal, and intranasal.

In one embodiment, the method of the present invention is a mono-therapy where the pharmaceutical compositions of the invention are administered alone. Accordingly, in this embodiment, the compound of the invention is the only pharmaceutically active ingredient in the pharmaceutical compositions or the only pharmaceutically active ingredient administered to the subject.

In another embodiment, the method of the invention is a co-therapy with one or more of other therapeutically active drugs or therapies known in the art for treating the desired diseases or indications. In one example, one or more other anti-proliferative or anticancer therapies are combined with the compounds of the invention. In another example, the compounds disclosed herein are co-administered with one or more of other anticancer drugs known in the art. Anticancer therapies that may be used in combination with the compound of the invention include surgery, radiotherapy (including, but not limited to, gamma-radiation, neutron beam radiotherapy, electron beam radiotherapy, proton therapy, brachytherapy, and systemic radioactive isotopes) and endocrine therapy. Anticancer agents that may be used in combination with the compounds of the invention include biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs (e.g., taxol and analogs thereof).

Examples of anti-cancer agents which can be co-administered with the disclosed angiogenesis inhibitors include abarelix, alitretinoin, allopurinol, altretamine, amifostine, anakinra, anastrozole, arsenic trioxide, asparaginase, azacitidine, BCG Live, bevacuzimab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, ctinomycin D, dalteparin sodium, darbepoetin alfa, dasatinib, daunorubicin, daunomycin, decitabine, denileukin, dexrazoxane, docetaxel, doxorubicin, dromostanlone propionate, eculizumab, Elliott's B Solution, epirubicin, epoetin alfa, erlotinib, estramustine, etoposide, exemestane, fentanyl citrate, Filgrastim, floxuridine (intraarterial), fludarabine, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, Interferon alfa-2b, irinotecan, lapatinib ditosylate, lenalidomide, letrozole, leucovorin, Leuprolide Acetate, levamisole, lomustine, CCNU, meclorethamine, nitrogen mustard, megestrol acetate, melphalan, L-PAM, mercaptopurine, 6-MP, mesna, methotrexate, methoxsalen, mitomycin C, mitotane, mitoxantrone, nandrolone phenpropionate, nelarabine, Nofetumomab, Oprelvekin, oprelvekin, oxaliplatin, paclitaxel, palifermin, pamidronate, panitumumab, pegademase, pegaspargase, Pegfilgrastim, Peginterferon alfa-2b, pemetrexed disodium, pentostatin, pipobroman, plicamycin, mithramycin, porfimer sodium, procarbazine, quinacrine, Rasburicase, Rituximab, Sargramostim, sorafenib, streptozocin, sunitinib, talc, tamoxifen, temozolomide, teniposide, VM-26, testolactone, thalidomide, thioguanine, 6-TG, thiotepa, topotecan, toremifene, Tositumomab, trastuzumab, tretinoin, ATRA, Uracil Mustard, valrubicin, vinblastine, vincristine, vinorelbine, vorinostat, and zoledronate.

In another example, the compounds disclosed herein are co-administered with one or more of other anti-angiogenesis drugs known in the art. Anti-angiogenesis agents that can be co-administered with the compounds of the invention include Dalteparin, Suramin, ABT-510, Combretastatin A4 Phosphate, Lenalidomide, LY317615 (Enzastaurin), Soy Isoflavone (Genistein; Soy Protein Isolate), Thalidomide, AMG-706, Anti-VEGF Antibody (Bevacizumab; Avastin™), AZD2171, Bay 43-9006 (Sorafenib tosylate), PI-88, PTK787/ZK 222584 (Vatalanib), SU11248 (Sunitinib malate), VEGF-Trap, XL184, ZD6474, ATN-161, EMD 121974 (Cilenigtide), Celecoxib, Angiostatin, Endostatin, Regranex, Apligraf, Paclitaxel, tetracyclines, clarithromycin, lasix, captopril, aspirin, Vitamin D3 analogs, retinoids, Imiquomod, Interferon alfa2a, Minocycline, copper peptide containing dressings, Lucentis™, ATG002, Pegaptanib Sodium, Tryptophanyl-tRNA synthetase, squalamine lactate, anecortave acetate, AdPEDF, AG-013958, JSM6427, TG100801, Veglin, ascorbic acid ethers (and their analogs), and Pamidronate.

When the compounds of the invention are combined with other anticancer drugs, they can be administered contemperaneously. As used herein, “administered contemporaneously” means that two substances are administered to a subject such that they are both biologically active in the subject at the same time. The exact details of the administration will depend on the pharmacokinetics of the two substances in the presence of each other, and can include administering one substance within a period of time of one another, e.g., 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances. Alternatively, the two agents can be administered separately, such that only one is biologically active in the subject at the same time.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene)derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fern), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “alkyl” means a straight or branched hydrocarbon radical having 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like. “Alkenyl” means an alkyl group with at least one double bond; and “alkynyl” means an alkyl group with at least one triple bond.

The term “cycloalkyl” means a monocyclic, bicyclic or tricyclic, saturated hydrocarbon ring having 3-10 ring carbon atoms and includes, for example, cyclopropyl, cyclobutyl, cyclopentyl, cyclohexyl, cycloheptyl, cyclooctyl, bicyclo[2.2.2]octyl, bicyclo[2.2.1]heptyl, spiro[4.4]nonane, adamantyl and the like. The term “amino,” as used herein, refers to a primary (—NH₂), secondary (—NHRₓ, tertiary (—NRₓR_{y}), or quaternary (—N⁺RₓR_{y}R_{z}) amine, where Rₓ, R_{y}, and R_{z} are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The term “aryl” means a carbocyclic aromatic radical having 6-14 ring atoms such as a phenyl group, a naphthyl group, an indanyl group or a tetrahydronaphthalene group. When substituted, an aryl group can be optionally substituted with 1-4 substituents. Exemplary substituents include alkyl, alkoxy, alkylthio, alkylsulfonyl, halogen, trifluoromethyl, dialkylamino, nitro, cyano, CO₂H, CONH₂, N-monoalkyl-substituted amido and N,N-dialkyl-substituted amido. Alternative substituents for an aryl group include the groups represented by R³.

The terms “halo” and “halogen” as used herein refer to an atom selected from fluorine, chlorine, bromine, and iodine.

The term “heteroaryl” means a 5-12-membered monocyclic or bicyclic heteroaromatic radical containing 0-4 heteroatoms selected from N, O, and S. A heteroaryl may optionally be fused to an saturated or unsaturated non-aromatic ring. Examples of heteroaryls include, for example, 2- or 3-thienyl, 2- or 3-furanyl, 2- or 3-pyrrolyl, 2-, 3-, or 4-pyridyl, 2-pyrazinyl, 2-, 4-, or 5-pyrimidinyl, 3- or 4-pyridazinyl, 1H-indol-6-yl, 1H-indol-5-yl, 1H-benzimidazol-6-yl, 1H-benzimidazol-5-yl, 2-, 4-, 5-, 6-, 7- or 8-quinazolinyl, 2-, 3-, 5-, 6-, 7- or 8-quinoxalinyl, 2-, 3-, 4-, 5-, 6-, 7- or 8-quinolinyl, 1-, 3-, 4-, 5-, 6-, 7- or 8-isoquinolinyl, 2-, 4-, or 5-thiazolyl, 2-, 3-, 4-, or 5-pyrazolyl, 2-, 3-, 4-, or 5-imidazolyl. When substituted, a heteroaryl can be optionally substituted with 1 to 4 substituents. Exemplary substituents include alkyl, alkoxy, alkylthio, alkylsulfonyl, halogen, trifluoromethyl, dialkylamino, nitro, cyano, CO₂H, CONH₂, N-monoalkyl-substituted amido and N,N-dialkyl-substituted amido, or by oxo to form an N-oxide. Alternative substituents for a heteroaryl group include the groups represented by R³.

The term “heterocyclyl” means a 4-, 5-, 6- and 7-membered saturated or partially unsaturated heterocyclic ring containing 1 to 4 heteroatoms independently selected from N, O, and S. Exemplary heterocyclyls include pyrrolidine, pyrrolidin-2-one, 1-methylpyrrolidin-2-one, piperidine, piperidin-2-one, 2-pyridone, 4-pyridone, piperazine, 1-(2,2,2-trifluoroethyl)piperazine, piperazin-2-one, 5,6-dihydropyrimidin-4-one, pyrimidin-4-one, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, oxazolidin-2-one, imidazolidin-2-one, imidazolidine-2,4-dione, tetrahydropyrimidin-2(1H)-one, morpholine, N-methylmorpholine, morpholin-3-one, 1,3-oxazinan-2-one, thiomorpholine, thiomorpholine 1,1-dioxide, tetrahydro-1,2,5-thiaoxazole 1,1-dioxide, tetrahydro-2H-1,2-thiazine 1,1-dioxide, hexahydro-1,2,6-thiadiazine 1,1-dioxide, tetrahydro-1,2,5-thiadiazole 1,1-dioxide, and isothiazolidine 1,1-dioxide. When substituted, a heterocyclyl can be optionally substituted with 1-4 substituents. Exemplary substituents include alkyl, haloalkyl and oxo. Alternative substituents for a heterocyclyl include the groups represented by R³.

Certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the compound has at least one chiral center, it is to be understood that the name or structure encompasses one enantiomer of compound free from the corresponding optical isomer, a racemic mixture of the compound and mixtures enriched in one enantiomer relative to its corresponding optical isomer.

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

When a disclosed compound has multiple chiral centers and the configuration at only some of the chiral centers is depicted by name or structure, it is to be understood that the name or structure at the chiral center(s) where the configuration is not designated, includes one configuration, equal amounts of both configurations or unequal amounts of both configurations.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Pharmaceutically acceptable basic/cationic salts include, the sodium, potassium, calcium, magnesium, diethanolamine, n-methyl-D-glucamine, L-lysine, L-arginine, ammonium, ethanolamine, piperazine and triethanolamine salts.

The invention is illustrated by the following examples which are not intended to be limiting in any way.

### EXEMPLIFICATION

### Examples of Synthetic Methods

Representative methods for the synthesis of Example Compounds and requisite intermediates are shown below. Intermediates whose synthesis is not described were commercially available or prepared by literature methods.

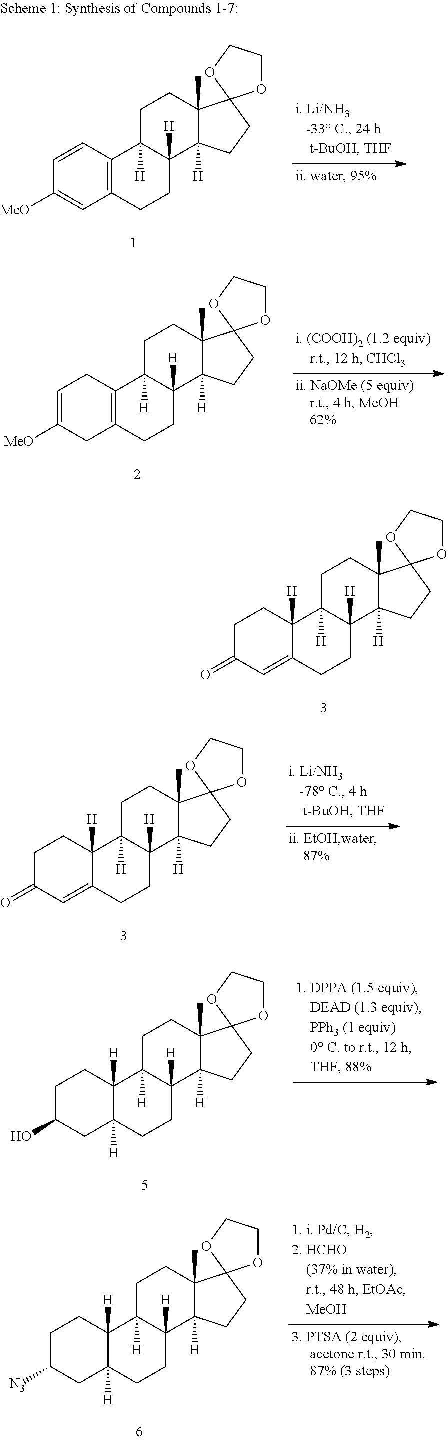

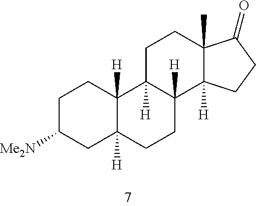

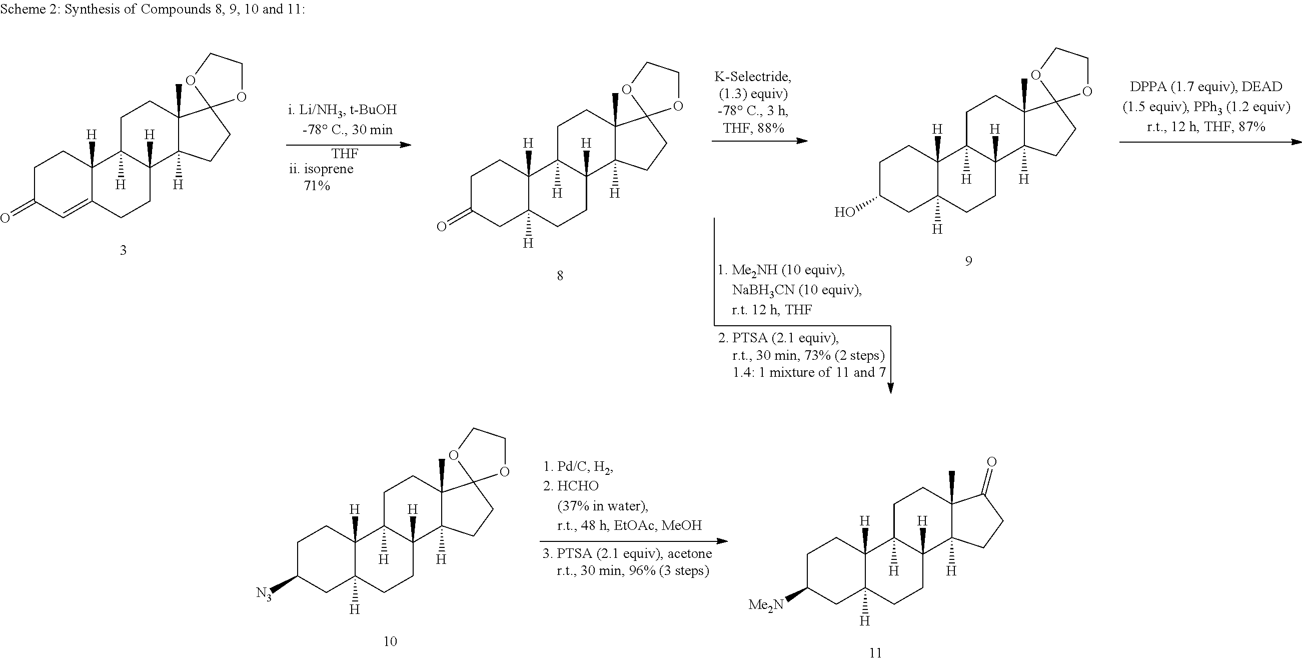

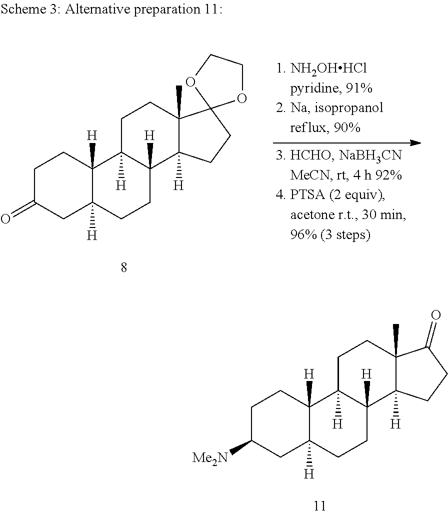

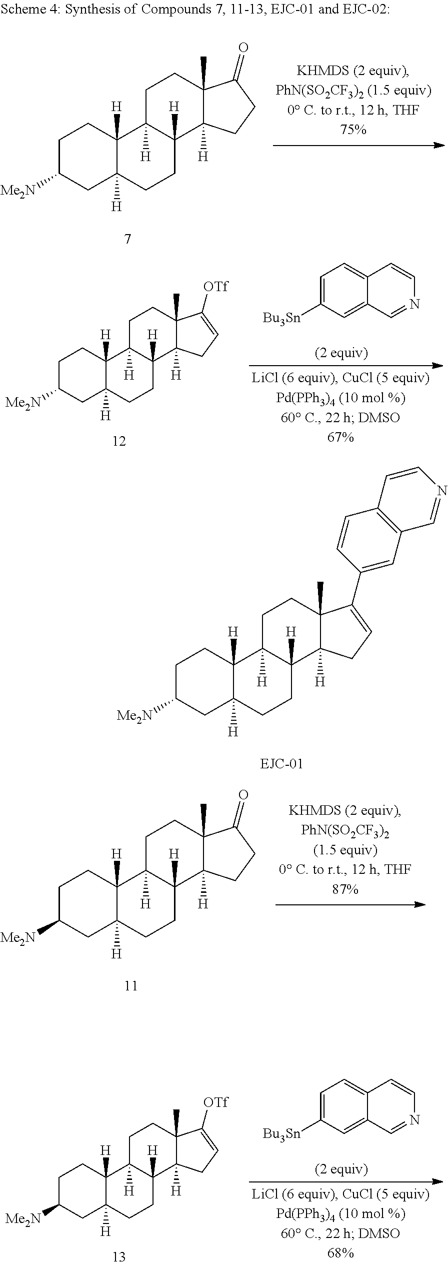

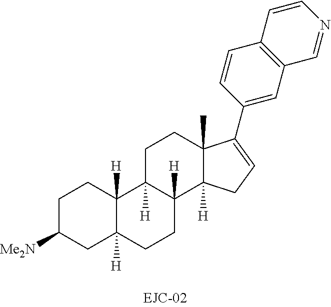

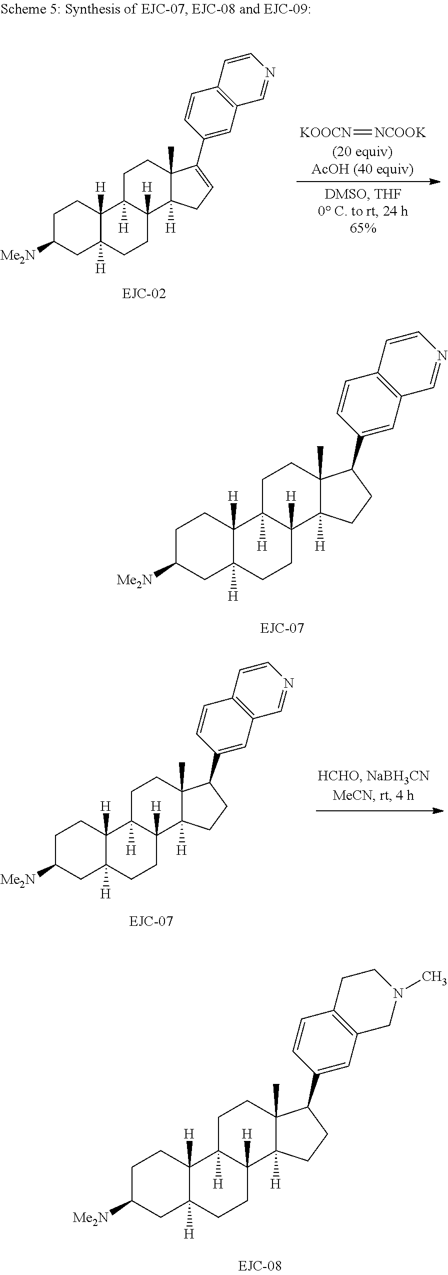

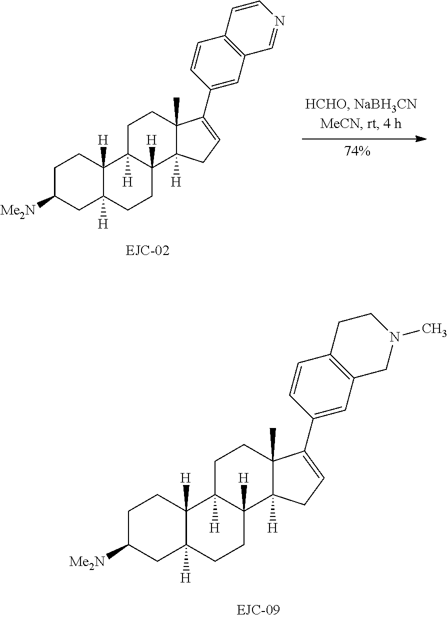

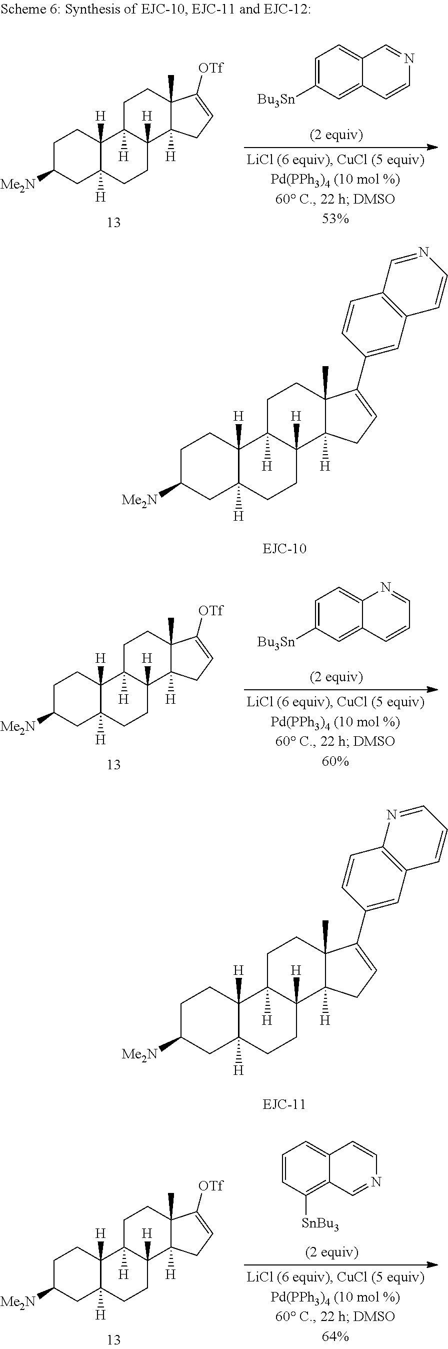

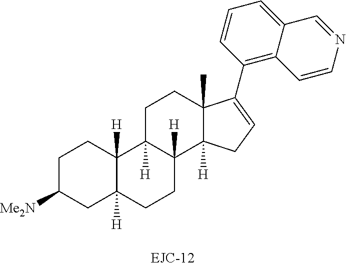

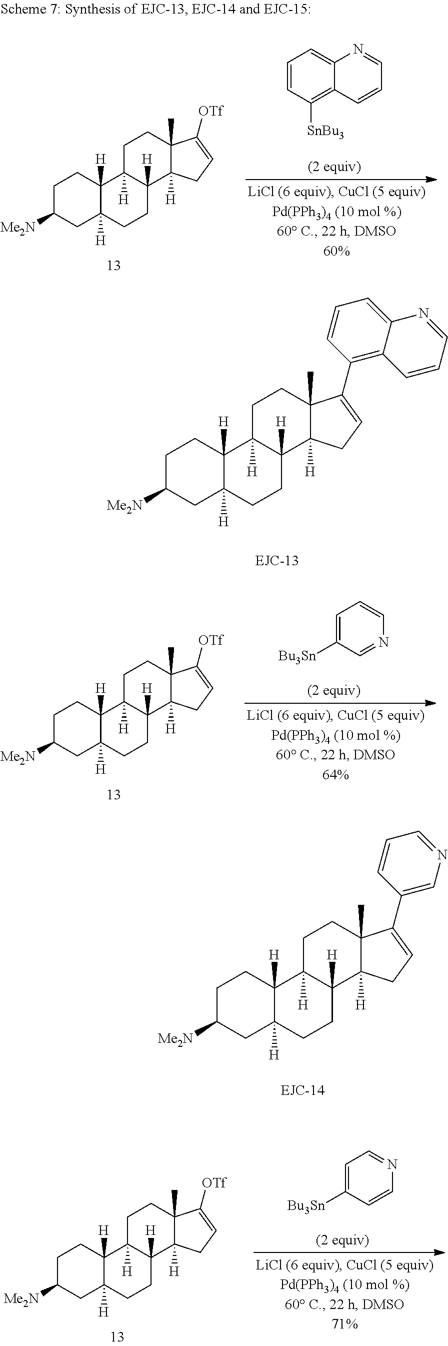

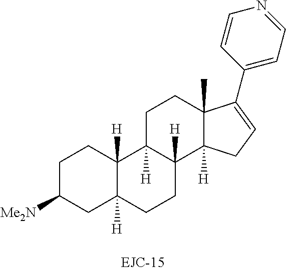

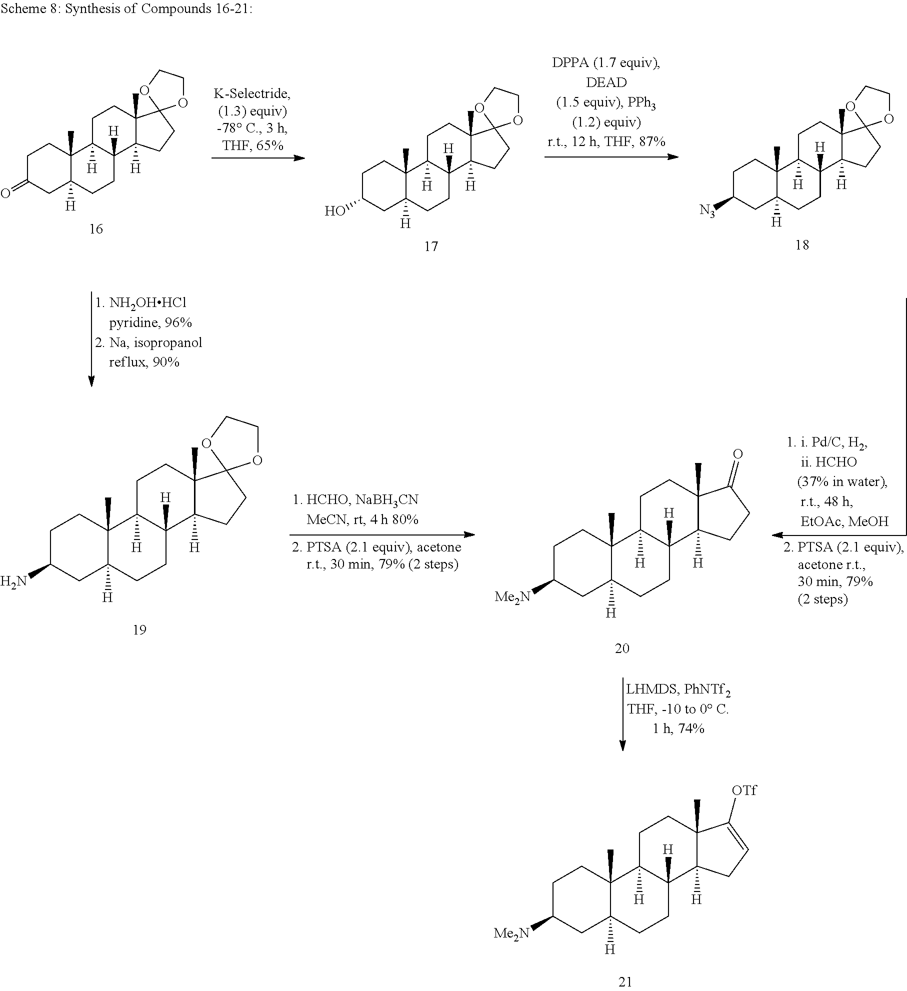

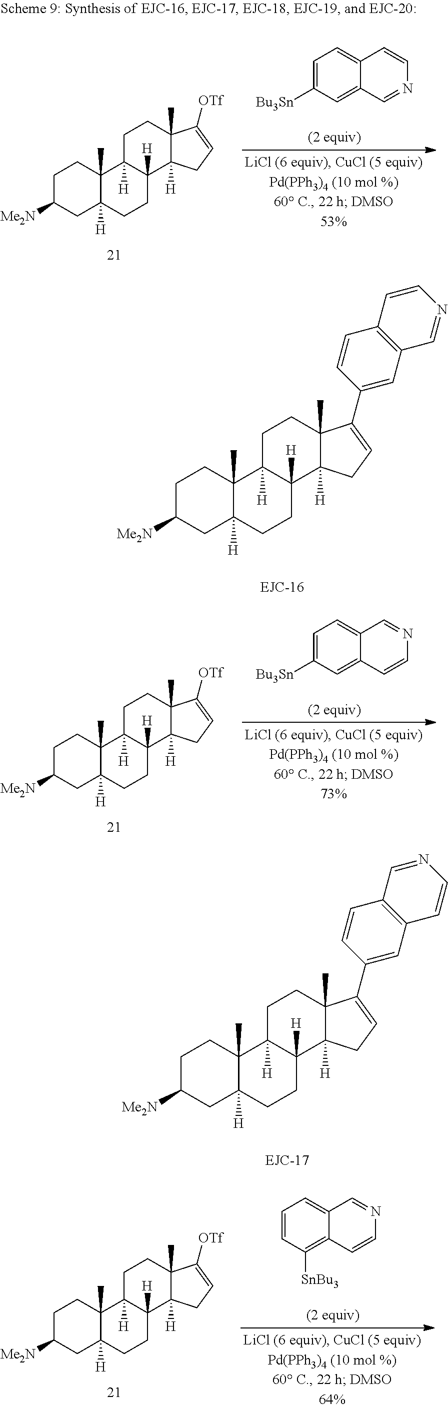

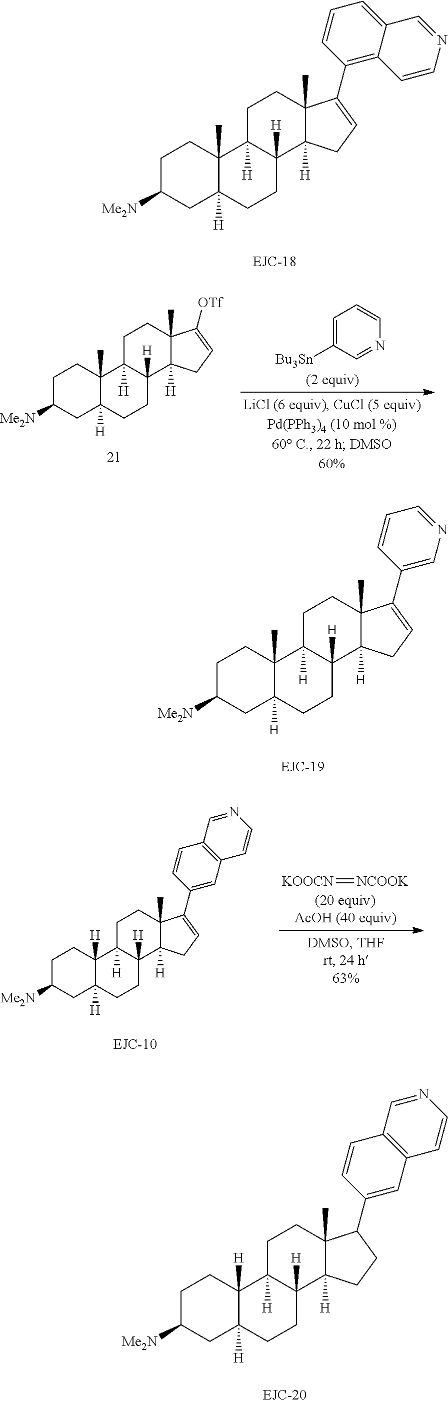

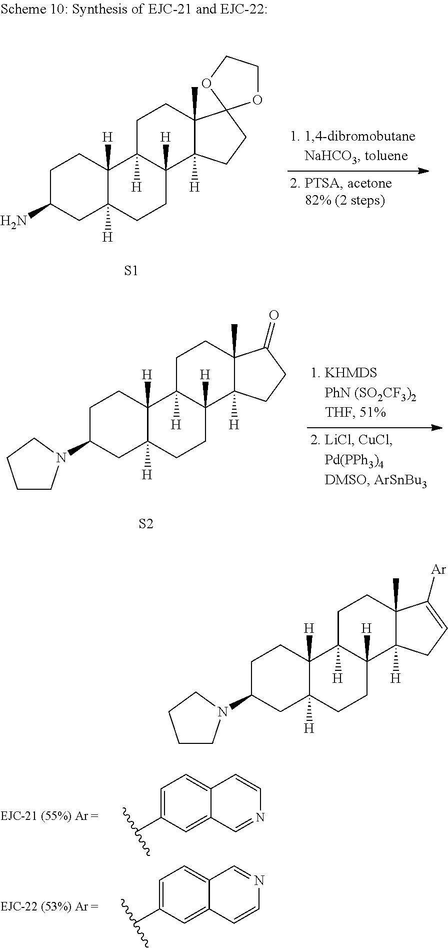

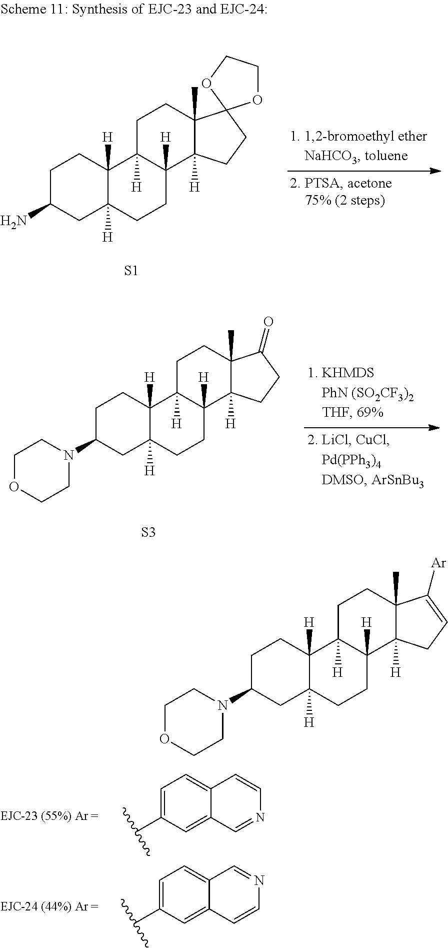

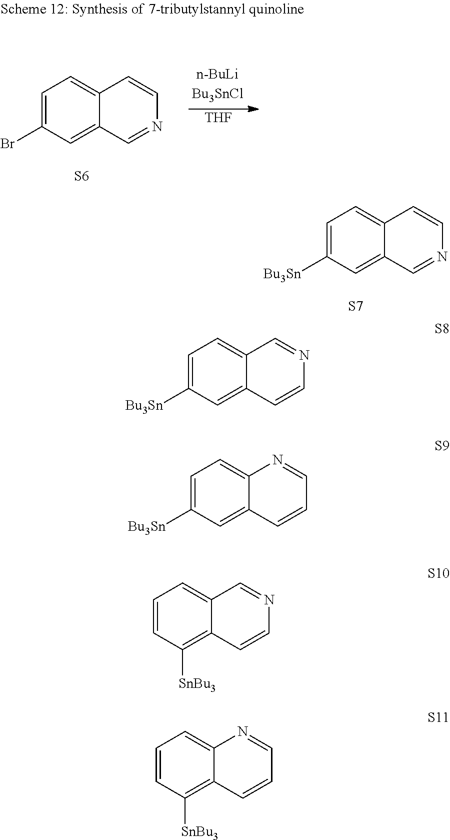

### Materials and Methods

Unless stated otherwise, reactions were performed in flame-dried glassware under a positive pressure of argon using freshly distilled dry solvents. Commercial grade reagents and solvents were used without further purification except as indicated below. MeOH was distilled over CaSO₄. Dichloromethane and 1,2-dichloroethane were distilled from calcium hydride. Toluene, Et₂O and THF were purified by Seco Solvent Systems. Thin-layer chromatography (TLC) was performed using E. Merck silica gel 60 F254 precoated plates (0.25 mm). Flash chromatography was performed using Baker silica gel (40 μm particle size). ¹H-NMR spectra were recorded on Varian Mercury 400 (400 MHz) or Unity/INOVA 500 (500 MHz) spectrometers and chemical shifts are reported in ppm from tetramethylsilane with the solvent resonance as internal standard (δ 7.26 ppm for CDCl₃). ¹³C NMR spectra were recorded on Varian Mercury 400 (100 MHz) or Unity/INOVA 500 (126 MHz) spectrometers with proton decoupling. Chemical shifts are reported in ppm from tetramethylsilane with the solvent as internal (δ 77.16 ppm for CDCl₃). IR spectra were recorded on Avatar 360 FT-IR spectrometer. Low-resolution and high-resolution mass spectral analyses were performed at the Harvard University Mass Spectrometry Center. Optical rotations were measured with a Perkin-Elmer 241 polarimeter at the indicated temperature with a sodium lamp (D line, 589 nM). Melting points (m.p.) are uncorrected and were recorded on a Büchi capillary melting point apparatus.

### Preparation of Enol Ether(+)-2.

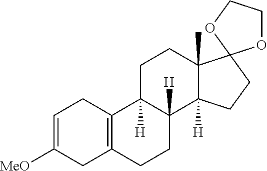

A round bottom three-necked flask was equipped with a dry ice condenser and a magnetic stir bar. The flask was flame-dried under Ar atmosphere and cooled to −78° C. The condenser was filled with dry ice and acetone, and ammonia (30 mL) was condensed at −78° C. To the NH₃(l) was added Li wire (5.21 cm, 234 mg, 33 mmol, 5 equiv) in five portions over 45 minutes. The Li/NH₃(l) was allowed to warm to −33° C. and stirred for another 35 min. Compound 1 (2.2 g, 6.7 mmol, 1 equiv) was dissolved in THF (15 mL), and t-BuOH (3.15 mL, 33.5 mmol, 5 equiv) was added. This solution was cannulated to the Li/NH₃(l) solution. The reaction mixture was stirred at −33° C. for 24 h. Subsequently, the reaction mixture was very carefully quenched with water at −33° C., and allowed to warm to room temperature. After evaporation of the NH₃(l), Et₂O was added (30 mL) followed by the addition of water (20 mL). The phases were separated and the aqueous phase was extracted with Et₂O (3×20 mL). The combined organic phases were dried (Na₂SO₄) and the solvent was evaporated to provide the product (2.1 g, 95%) that could be used in the next step without further purification. Compound 2 was previously reported in literature. ¹H NMR (500 MHz, CDCl₃) δ 4.64 (s, 1H), 4.04-3.79 (m, 4H), 3.55 (s, 3H), 3.00-2.77 (m, 1H), 2.72-2.57 (m, 2H), 2.52 (ddd, J=18.91, 9.35, 7.00 Hz, 1H), 2.13-2.04 (m, 1H), 2.00 (ddd, J=14.46, 11.60, 2.62 Hz, 1H), 1.92-1.84 (m, 2H), 1.82 (dd, J=8.90, 5.61 Hz, 1H), 1.79 (dd, J=8.82, 5.46 Hz, 1H), 1.76-1.66 (m, 2H), 1.65-1.58 (m, 1H), 1.56 (s, 1H), 1.55-1.51 (m, 1H), 1.47-1.40 (m, 1H), 1.39-1.26 (m, 2H), 1.26-1.14 (m, 1H), 0.87 (s, 3H); ¹³C NMR (125 MHz, CDCl₃) δ 152.8, 128.1, 125.1, 119.6, 90.8, 65.4, 64.7, 54.0, 49.4, 46.4, 45.3, 39.3, 34.5, 34.3, 31.2, 30.7, 28.5, 26.6, 25.4, 22.4, 14.6.

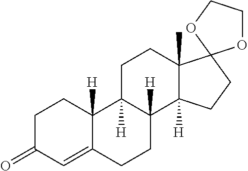

### Preparation of Enone (+)-3.

Compound 2 (1.8 g, 5.4 mmol, 1 equiv) was dissolved in CHCl₃ (27.5 mL) and (COOH)₂.2H₂O (824 mg, 6.5 mmol, 1.2 equiv) was added. The reaction mixture was stirred at room temperature for 12 h. Saturated NaHCO₃ solution (25 mL) was added, the phases were separated and the aqueous phase was extracted with CHCl₃ (3×15 mL). The combined organic phases were dried (Na₂SO₄) and the solvent was evaporated. The crude product was dissolved in MeOH (27 mL) and MeONa (1.45 g, 27 mmol, 5 equiv) was added. The reaction was stirred at room temperature for 4 h. Saturated NH₄Cl solution was added (5 mL) and the MeOH was evaporated. Et₂O was added (25 mL) and the solution was washed with saturated NH₄Cl solution (15 mL). The phases were separated and the aqueous phase was extracted with Et₂O (2×10 mL). The combined organic phases were dried (Na₂SO₄) and the solvent was evaporated. The residue was purified by flash chromatography (10 to 30% EtOAc in hexanes on SiO₂) to afford the product (1.72 g, 62%). Compound 3 was previously reported in literature. ¹H NMR (500 MHz, CDCl₃) δ 5.78 (s, 1H), 4.03-3.68 (m, 4H), 2.43 (ddd, J=14.65, 3.76, 2.46 Hz, 1H), 2.39-2.32 (m, 1H), 2.25 (dd, J=4.82, 3.85 Hz, 1H), 2.23-2.21 (m, 1H), 2.19 (t, J=5.42, 5.42 Hz, 1H), 2.04 (dd, J=11.52, 7.96 Hz, 1H), 1.96 (ddd, J=14.53, 11.68, 3.15 Hz, 1H), 1.88-1.72 (m, 3H), 1.65 (dddd, J=12.66, 9.91, 7.09, 3.14 Hz, 1H), 1.61-1.46 (m, 2H), 1.46-1.34 (m, 2H), 1.35-1.15 (m, 4H), 1.03 (ddd, J=25.08, 13.27, 4.04 Hz, 1H), 0.87 (s, 3H), 0.86-0.79 (m, 1H); ¹³C NMR (125 MHz, CDCl₃) δ 200.0, 166.8, 124.7, 119.3, 65.4, 64.7, 49.5, 49.2, 46.0, 42.7, 40.8, 36.7, 35.6, 34.2, 30.6, 30.5, 26.8, 26.1, 22.6, 14.5.

### Preparation of Alcohol (+)-5.

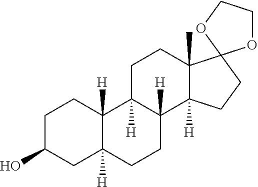

A round bottom three-necked flask was equipped with a dry ice condenser and a magnetic stir bar. The flask was flame-dried under Ar atmosphere and cooled to −78° C. The condenser was filled with dry ice and acetone, and ammonia (33 mL) was condensed at −78° C. To the NH₃(l) was added Li wire (5.6 cm, 254 mg, 36 mmol, 10 equiv) in five portions over 45 minutes and stirred for another 35 min. Compound 3 (1.2 g, 3.6 mmol, 1 equiv) was dissolved in THF (13 mL), and t-BuOH (3.44 mL, 36 mmol, 10 equiv) was added. This solution was cannulated to the Li/NH₃(l) solution. The reaction mixture was stirred at −78° C. for 4 h. Subsequently, EtOH (2.1 mL, 36 mmol, 10 equiv) was added very carefully and the reaction mixture was stirred for an additional hour. The reaction mixture quenched with water at −78° C., and allowed to warm to room temperature. After evaporation of the NH₃(l), Et₂O was added (30 mL) followed by the addition of excess water (20 mL). The phases were separated and the aqueous phase was extracted with Et₂O (3×20 mL). The combined organic phases were dried (Na₂SO₄) and the solvent was evaporated to provide the product (1.05 g, 87%) that could be used in the next step without further purification. NMR (500 MHz, CDCl₃) δ 4.11-3.69 (m, 4H), 3.62-3.52 (m, 1H), 2.11-1.85 (m, 4H), 1.88-1.81 (m, 1H), 1.80-1.72 (m, 2H), 1.68-1.56 (m, 4H), 1.56-1.47 (m, 1H), 1.45-1.31 (m, 2H), 1.29-1.11 (m, 3H), 1.10-0.94 (m, 4H), 0.94-0.87 (m, 1H), 0.87-0.85 (m, 1H), 0.83 (s, 3H), 0.75-0.54 (m, 1H); ¹³C NMR (125 MHz, CDCl₃) δ 119.7, 70.7, 65.3, 64.7, 49.7, 48.0, 46.4, 46.2, 43.6, 41.7, 41.3, 36.0, 34.3, 33.7, 30.9, 30.5, 28.6, 25.6, 22.6, 14.5. IR (neat, cm⁻¹) 3389 (bs), 2969 (w), 2909 (s), 2819 (m), 1447 (w), 1309 (w), 1165 (w), 1099 (w), 1051 (m), 1027 (w); [α]_{D}²⁰=+5.4 (c 1.00, CHCl₃); HRMS (ESI-MS) calcd. for C₂₀H₃₃NO₃ [(M+H)⁺] 321.2424, found 324.2440.

### Preparation of Azide (+)-6.

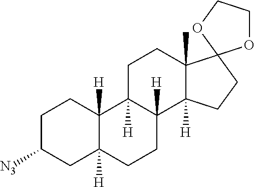

To a solution of PPh₃ (471 mg, 1.8 mmol, 1 equiv) in THF (7 mL) was added diethyl azodicarboxylate (306 pit, 1.95 mmol, 1.3 equiv) at 0° C. and the solution was stirred for 10 minutes. To this solution was added a compound 5 (500 mg, 1.5 mmol, 1 equiv) in THF (4 mL). After stirring for 10 minutes, a solution of diphenylphosphoryl azide (483 μL, 2.25 mmol, 1.5 equiv) was added. The reaction mixture was allowed to warm to room temperature and stirred for 10 h. Then the solvent was evaporated and the residue was purified by flash chromatography (2% EtOAc in hexanes on SiO₂) to afford the product (516 mg, 88%). ¹H NMR (500 MHz, CDCl₃) δ 4.08-3.63 (m, 4H), 1.97 (ddd, J=14.58, 11.69, 3.16 Hz, 1H), 1.94-1.85 (m, 1H), 1.83-1.60 (m, 5H), 1.61-1.40 (m, 5H), 1.37 (ddd, J=12.54, 4.03, 2.80 Hz, 1H), 1.34-1.26 (m, 1H), 1.26-1.17 (m, 4H), 1.16-0.89 (m, 5H), 0.84 (s, 3H), 0.78-0.65 (m, 1H); ¹³C NMR (125 MHz, CDCl₃) δ 119.7, 65.4, 64.7, 58.1, 49.6, 47.9, 47.0, 46.2, 41.7, 37.6, 36.9, 34.3, 33.5, 30.8, 30.3, 30.0, 25.2, 24.6, 22.6, 14.5; IR (neat, cm⁻¹) 2914 (s), 2862 (m), 2092 (s), 1298 (w), 1257 (w), 1170 (w), 1106 (w), 1035 (w); [α]_{D}²⁰=+5.4 (c 1.00, CHCl₃); HRMS (ESI-MS) calcd. for C₂₀H₃₂NO₃ [(M+H)⁺] 346.2489, found 346.2487.

### Preparation of Ketal of Dimethylamine (+)-7.

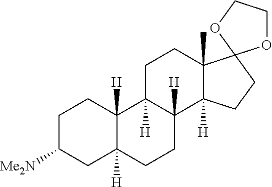

A round bottom flask was charged with 10% Pd/C (16 mg, 10%) and MeOH (2.2 mL). The slurry was purged with H₂ gas for 5 minutes. A solution of compound 6 (160 mg, 0.44 mmol) in EtOAc (2.2 mL) was added and the reaction mixture was stirred for 4 h under H₂ atmosphere at room temperature. Subsequently, 37% HCHO solution in water (660 μL, 20 equiv) was added and the reaction mixture was stirred for 48 h. The mixture was filtered through Celite and the solvent was evaporated. The crude product was used in the next step without further purification. ¹H NMR (500 MHz, CDCl₃) δ 4.05-3.67 (m, 4H), 3.06-2.85 (m, 1H), 2.37 (s, 6H), 2.09-2.00 (m, 1H), 1.94 (ddd, J=14.50, 11.70, 3.10 Hz, 1H), 1.87-1.80 (m, 1H), 1.79-1.70 (m, 2H), 1.68-1.56 (m, 3H), 1.55-1.47 (m, 2H), 1.47-1.36 (m, 2H), 1.36-1.27 (m, 2H), 1.27-1.07 (m, 3H), 1.07-0.92 (m, 4H), 0.89-0.83 (m, 1H), 0.81 (s, 3H), 0.80-0.71 (m, 1H); ¹³C NMR (125 MHz, CDCl₃) δ 119.7, 65.3, 64.7, 62.2, 49.5, 48.0, 47.5, 46.2, 46.2, 43.7, 41.9, 36.2, 34.4, 33.9, 30.8, 30.2, 29.0, 25.3, 24.2, 22.6, 14.5; IR (neat, cm⁻¹) 2908 (s), 2868 (m), 1677 (w), 1537 (w), 1549 (w), 1502 (w), 1450 (w), 1160 (w) 1030 (w); [α]_{D}²⁰=+1.6 (c 1.00, CHCl₃); HRMS (ESI-MS) calcd. for C₂₂H₃₈NO₂ [(M+H)⁺] 348.2897, found 348.2900.

Alternative method for dimethylation (Method B): The crude amine (0.26 mmol) was dissolved in MeCN (2.6 mL) and 37% aqueous formaldehyde (2.3 mL, 120 equiv) was added followed by the addition of 30% aqueous AcOH solution (390 μL, 7.7 equiv). NaBH₃CN (72 mg, 1.17 mmol, 4.5 equiv) was added in three portions over three hours and the reaction was stirred for 4 hours. The reaction mixture was quenched with NaHCO₃ solution, extracted with Et₂O and EtOAc, the combined organic phases were dried (Na₂SO₄), and the solvent was evaporated. The crude product was used in the next step without further purification.

### Preparation of Dimethylaminoketone (+)-7.

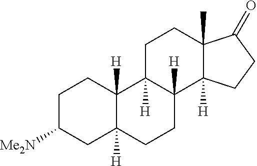

The crude product from the previous reaction (0.44 mmol) was dissolved in acetone (4.4 mL), and PTSA.H₂O (167 mg, 0.88 mmol, 2 equiv) was added. The reaction was stirred at room temperature for 30 minutes. The acetone was evaporated and residue was dissolved in water (5 mL). The aqueous phase was washed with Et₂O (2×2 mL). The aqueous phase was treated with 2 M NaOH solution and the pH was adjusted to 11. The aqueous phase was extracted with benzene (4×3 mL). The combined organic phases were dried (Na₂SO₄) and the solvent was evaporated. The residue was purified by flash chromatography (10% MeOH in CHCl₃ on SiO₂ that was pretreated with 1% Et₃N in CHCl₃) to afford the product (116 mg, 87% for three steps). ¹H NMR (500 MHz, CDCl₃) δ 2.42 (ddd, J=19.10, 8.83, 0.82 Hz, 1H), 2.22 (s, 6H), 2.11-1.98 (m, 3H), 1.92 (ddd, J=12.40, 9.06, 3.26 Hz, 1H), 1.87-1.80 (m, 2H), 1.80-1.71 (m, 2H), 1.64-1.53 (m, 2H), 1.49 (ddd, J=12.40, 9.06, 3.26 Hz, 1H), 1.36-1.13 (m, 4H), 1.13-0.99 (m, 5H), 0.86 (s, 4H), 0.91-0.78 (m, 2H); ¹³C NMR (125 MHz, CDCl₃) δ 221.7, 61.4, 50.9, 48.4, 48.1, 48.0, 44.0, 41.1, 37.4, 36.4, 36.0, 33.9, 31.8, 30.0, 29.6, 25.0, 24.4, 21.8, 14.0; IR (neat, cm⁻¹) 2914 (s), 2850 (m), 2763 (w), 1724 (s), 1648 (w), 1549 (w), 1456 (w), 1263 (w); [α]_{D}²⁰=+60.5 (c 1.00, CHCl₃); HRMS (ESI-MS) calcd. for C₂₀H₃₄NO₂ [(M+H)⁺] 304.2634, found 304.2617.

### Preparation of Triflate (+)-12.

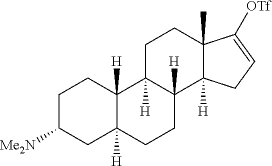

Ketone 7 (104 mg, 0.34 mmol, 1 equiv) was dissolved in THF (1.1 mL) and a 0.5 M solution of KHMDS (1.37 mL, 0.68 mmol, 2 equiv) in THF was added at 0° C. The reaction mixture was stirred for 30 minutes after which a 0.5 M solution of PhN(SO₂CF₃)₂ (1.3 mL, 0.51 mmol, 1.5 equiv) in THF was added. The reaction mixture was allowed to warm to room temperature and stirred for 10 h. Water (4 mL) was added and the phases were separated. The aqueous phase was extracted with Et₂O (2 mL) and EtOAc (2×2 mL), the combined organic phases were dried (Na₂SO₄) and the solvent was evaporated. The residue was purified by flash chromatography (2% MeOH in CHCl₃ on SiO₂ that was pretreated with 1% Et₃N in CHCl₃) to afford the product (111 mg, 75%). ¹H NMR (500 MHz, CDCl₃) δ 5.54 (dd, J=3.20, 1.63 Hz, 1H), 2.29 (s, 6H), 2.19 (ddd, J=14.95, 6.38, 3.35 Hz, 1H), 2.15 (s, 1H), 2.09-2.00 (m, 1H), 1.96 (ddd, J=14.93, 11.25, 1.59 Hz, 1H), 1.90-1.78 (m, 2H), 1.69 (ddd, J=12.56, 4.30, 2.32 Hz, 1H), 1.66-1.52 (m, 3H), 1.52-1.45 (m, 1H), 1.44-1.37 (m, 2H), 1.37-1.28 (m, 1H), 1.28-1.18 (m, 2H), 1.12 (dd, J=30.24, 13.36 Hz, 2H), 1.06-0.98 (m, 2H), 0.96 (s, 3H), 0.93-0.76 (m, 2H); ¹³C NMR (125 MHz, CDCl₃) δ 159.7, 118.7 (q), 114.5, 61.4, 53.9, 48.6, 48.1, 45.2, 44.0, 39.5, 37.4, 36.5, 33.8, 32.9, 30.0, 29.6, 28.5, 25.1, 24.2, 15.5; IR (neat, cm⁻¹) 2921 (s), 2853 (m), 1490 (w), 1416 (w), 1370 (w), 1262 (w), 1205 (s), 1142 (s), 1062 (w); [α]_{D}²⁰=+25.5 (c 1.00, CHCl₃); HRMS (ESI-MS) calcd. for C₂₁H₃₃F₃NO₃S [(M+H)⁺] 436.2127, found 436.2110.

### Preparation of (+)-EJC-01.

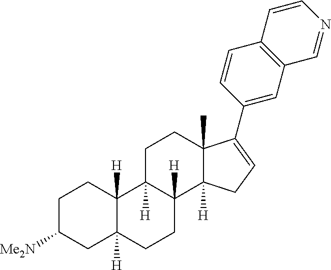

A Schlenk tube was fitted with a stir bar and flame-dried under N₂ atmosphere. It was charged with triflate 12 (21 mg, 0.048 mmol, 1 equiv), flame-dried LiCl (12 mg, 0.28 mmol, 6 equiv), CuCl (24 mg, 0.24 mmol, 5 equiv), and Pd(PPh₃)₄ (5.5 mg, 0.0048, 10 mol %). The Schlenk tube was evacuated and refilled with N₂ twice and DMSO (1.9 mL) was added followed by the addition of 7-tributylstannyl isoquinoline (40 mg, 0.096 mmol, 2 equiv). The reaction mixture was degassed by the freeze-pump thaw process (3 cycles, −78° C. to room temperature) and was stirred at room temperature for 1 h. Subsequently, it was warmed to 60° C. and kept at this temperature for 20 hours. The reaction mixture was poured on brine (8.6 mL) and aqueous NH₃ (5%, 1.4 mL). The mixture was extracted with Et₂O (4×5 mL). The combined organic phases were dried (Na₂SO₄) and the solvent was evaporated. The residue was purified on preparative TLC (500 μm, w/UV254; 1% MeOH in CHCl₃ containing 0.3% aqueous NH₃) to afford the product (13 mg, 67%). ¹H NMR (500 MHz, CDCl₃) δ 9.21 (s, 1H), 8.47 (s, 1H), 7.89 (s, 1H), 7.77-7.71 (m, 2H), 7.60 (d, J=5.27 Hz, 1H), 6.08 (dd, J=2.90, 1.62 Hz, 1H), 2.80 (s, 6H), 2.26 (ddd, J=15.70, 6.66, 3.40 Hz, 1H), 2.15-2.09 (m, 1H), 2.04 (ddd, J=15.60, 11.88, 1.33 Hz, 1H), 1.91 (dd, J=32.21, 25.98 Hz, 2H), 1.72 (dd, J=18.79, 8.05 Hz, 3H), 1.69-1.59 (m, 3H), 1.60-1.51 (m, 2H), 1.43-1.32 (m, 3H), 1.30-1.20 (m, 3H), 1.10 (s, 3H), 1.04 (m, 1H), 0.97-0.80 (m, 2H); ¹³C NMR (125 MHz, CDCl₃) δ 153.9, 151.5, 140.8, 137.2, 135.3, 131.4, 129.9, 128.5, 126.5, 124.2, 121.3, 64.0, 56.4, 47.8, 47.5, 46.9, 43.0, 42.7, 40.0, 35.5, 34.6, 33.6, 31.6, 30.2, 27.3, 25.8, 23.4, 16.9; IR (neat, cm⁻¹) 3028 (w), 2927 (s), 2856 (m), 1639 (w), 1593 (w), 1452 (m), 1396 (w), 1250 (w), 1209 (w), 1169 (w), 1113 (w), 1030 (w); [α]_{D}²⁰=+29.7 (c 1.00, CHCl₃); HRMS (ESI-MS) calcd. for C₂₉H₃₉N₂ [(M+H)⁺] 415.3107, found 415.3105.

### Preparation of Ketone (+)-8.

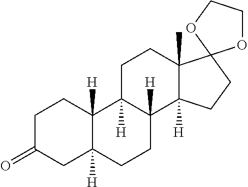

A round bottom three-necked flask was equipped with a dry ice condenser and a magnetic stir bar. The flask was flame-dried under Ar atmosphere and cooled to −78° C. The condenser was filled with dry ice and acetone, and ammonia (5.3 mL) was condensed at −78° C. To the NH₃(l) was added Li wire (0.17 cm, 7.8 mg, 1.1 mmol, 2.1 equiv) in 2 portions over 15 minutes. The Li/NH₃(l) stirred for another 30 min. Compound 3 (176 mg, 0.53 mmol, 1 equiv) was dissolved in THF (1.7 mL), and t-BuOH (106 μL, 1.1 mmol, 2.1 equiv) was added. This solution was cannulated to the Li/NH₃(l) solution. The reaction mixture was stirred at −78° C. for 30 min. Subsequently, the reaction mixture was carefully quenched with isoprene, and allowed to warm to room temperature. After evaporation of the NH₃(l), Et₂O was added (5 mL) followed by the addition of water (5 mL). The phases were separated and the aqueous phase was extracted with diethyl ether (3×3 mL). The combined organic phases were dried (Na₂SO₄) and the solvent was evaporated. The residue was purified by flash chromatography (2% EtOAc in hexanes on SiO₂) to afford the product (126 mg, 71%). ¹H NMR (500 MHz, CDCl₃) δ 4.08-3.64 (m, 4H), 2.49-2.31 (m, 1H), 2.31-2.21 (m, 3H), 2.06 (t, J=13.51, 13.51 Hz, 1H), 2.00-1.92 (m, 1H), 1.84-1.72 (m, 2H), 1.72-1.60 (m, 3H), 1.55 (dt, J=13.03, 13.00, 4.27 Hz, 1H), 1.49-1.31 (m, 3H), 1.31-1.02 (m, 6H), 0.93 (ddd, J=16.58, 13.31, 3.83 Hz, 1H), 0.85 (s, 3H), 0.72 (ddd, J=13.60, 10.08, 4.06 Hz, 1H); ¹³C NMR (125 MHz, CDCl₃) δ 211.9, 119.5, 65.4, 64.7, 49.5, 48.8, 47.7, 46.2, 46.0, 43.8, 41.5, 41.5, 34.3, 34.1, 30.8, 30.7, 30.1, 25.8, 22.6, 14.5. IR (neat, cm⁻¹) 2957 (s), 2921 (s), 2861 (m), 1711 (s), 1447 (w), 1375 (w), 1303 (w), 1171 (w), 1105 (w), 1051 (w); [α]_{D}²⁰=+17.8 (c 1.00, CHCl₃); HRMS (ESI-MS) calcd. for C₂₀H₃₄NO₃ [(M+NH₄)⁺] 336.2533, found 336.2535.

### Preparation of Alcohol (−)-9.

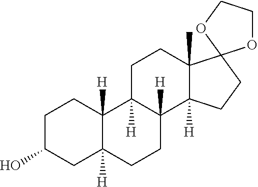

Compound 8 (116 mg, 0.34 mmol, 1 equiv) was dissolved in THF (3.4 mL) and the solution was cooled to −78° C. K-Selectride (1 M, 440 μL, 0.44 mmol, 1.3 equiv) was added and the reaction was stirred for 3 h. NaOH solution (2 M in water, 1 mL) was added followed by the addition of Et₂O (3 mL) and water (2 mL). The mixture was allowed to warm to room temperature and stirred for four hours. The phases were separated and the aqueous phase was extracted with EtOAc (2×3 mL). The combined organic phases were dissolved in Et₂O (5 mL) and NaOH solution (2 M in water) was added. The mixture was stirred for 12 hours. Subsequently the phases were separated, the aqueous phase was extracted with EtOAc (3×5 mL), the combined organic phases were dried (Na₂SO₄) and the solvent was evaporated. The product was purified using MPLC chromatography (20% EtOAc to 40% EtOAc) to provide the product (101 mg, 88%). ¹H NMR (500 MHz, CDCl₃) δ 4.19-4.01 (m, 1H), 3.96-3.76 (m, 4H), 2.48-2.10 (m, 1H), 1.96 (ddd, J=14.60, 11.70, 3.14 Hz, 1H), 1.84-1.72 (m, 2H), 1.74-1.59 (m, 4H), 1.58-1.31 (m, 5H), 1.30-1.08 (m, 4H), 1.10-0.86 (m, 4H), 0.84 (s, 3H), 0.75-0.68 (m, 1H); ¹³C NMR (125 MHz, CDCl₃) δ 119.7, 66.6, 65.3, 64.7, 49.7, 48.1, 47.3, 46.2, 41.7, 40.8, 36.2, 34.3, 33.8, 33.2, 30.9, 30.4, 25.3, 23.9, 22.6, 14.5; IR (neat, cm⁻¹) 3486, 2978 (m), 2908 (s), 2862 (m), 1380 (w), 1304 (w), 1246 (s), 1100 (s), 1047 (w); [α]_{D}²⁰=−1.5 (c 1.00, CHCl₃); HRMS (ESI-MS) calcd. for C₂₀H₃₃O₃ [(M+H)⁺] 321.2424, found 321.2433.

### Preparation of Azide (−)-10.

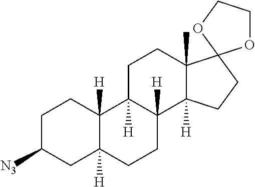

To a solution of PPh₃ (101 mg, 0.38 mmol, 1.3 equiv) in THF (1.4 mL) was added diethyl azodicarboxylate (70 μL, 0.44 mmol, 1.5 equiv) at 0° C., and the solution was stirred for 10 minutes. To this solution was added a compound 8 (100 mg, 0.30 mmol, 1 equiv) in THF (0.8 mL). After stirring for 10 minutes, a solution of diphenylphosphoryl azide (109 μL, 0.50 mmol, 1.7 equiv) was added. The reaction mixture was allowed to warm to room temperature and stirred for 10 h. Then the solvent was evaporated and the residue was purified by flash chromatography (2% EtOAc in hexanes on SiO₂) to afford the product (93 mg, 87%). ¹H NMR (500 MHz, CDCl₃) δ 4.14-3.59 (m, 4H), 3.32-3.14 (m, 1H), 1.98 (m, 3H), 1.89-1.81 (m, 1H), 1.80-1.70 (m, 2H), 1.65 (m, 3H), 1.52 (dt, J=13.16, 13.01, 4.26 Hz, 1H), 1.46-1.31 (m, 2H), 1.31-1.15 (m, 2H), 1.14-0.99 (m, 5H), 0.99-0.84 (m, 2H), 0.83 (s, 3H), 0.67 (m, 2H); ¹³C NMR (125 MHz, CDCl₃) δ 119.6, 65.3, 64.7, 60.0, 49.6, 47.9, 46.3, 46.2, 41.6, 41.6, 39.4, 34.3, 33.5, 32.1, 30.8, 30.3, 28.8, 25.5, 22.6, 14.5; IR (neat, cm⁻¹) 2926 (s), 2850 (s), 2092 (s), 1450 (w), 1304 (w), 1211 (w), 1158 (w), 1094 (w), 1030 (w); [α]_{D}²⁰=−7.8 (c 1.00, CHCl₃); HRMS (ESI-MS) calcd. for C₂₀H₃₂N₃O₂ [(M+H)⁺] 346.2489, found 346.2494.

### Preparation of Ketal of Dimethylaminoketone (+)-11.

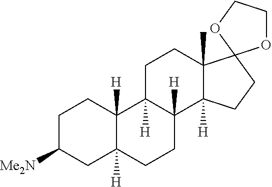

A round bottom flask was charged with 10% Pd/C (9.6 mg, 10%) and MeOH (1.3 mL). The slurry was purged with H₂ gas for 5 minutes. A solution of compound 10 (96 mg, 0.26 mmol, 1 equiv) in EtOAc (1.3 mL) was added and the reaction mixture was stirred for 4 h under H₂ atmosphere at room temperature. Subsequently, 37% HCHO solution in water (395 μL, 20 equiv) was added and the reaction mixture was stirred for 48 h. The reaction mixture was filtered through Celite and the solvent was evaporated. The crude product was used in the next step without further purification. ¹H NMR (500 MHz, CDCl₃) δ 4.05-3.67 (m, 4H), 2.38 (s, 6H), 2.06-1.89 (m, 3H), 1.87-1.70 (m, 3H), 1.70-1.58 (m, 3H), 1.53 (dt, J=13.06, 13.04, 4.27 Hz, 1H), 1.45-1.29 (m, 3H), 1.27-1.13 (m, 3H), 1.11-0.96 (m, 5H), 0.95-0.86 (m, 1H), 0.83 (s, 3H), 0.73-0.56 (m, 2H); ¹³C NMR (125 MHz, CDCl₃) δ 119.6, 65.3, 64.7, 63.6, 49.6, 48.0, 46.8, 46.2, 42.0, 41.6, 40.9, 35.6, 34.3, 33.8, 30.8, 30.1, 29.2, 28.3, 25.5, 22.6, 14.5; IR (neat, cm⁻¹) 2911 (w), 2854 (w), 1596 (w), 1442 (w), 1377 (w), 1307 (w), 1168 (w), 1108 (w), 1054 (w), 1034 (w); [α]_{D}²⁰=+17.8 (c 1.00, CHCl₃); HRMS (ESI-MS) calcd. for C₂₂H₃₈NO₂ [(M+H)⁺] 348.2897, found 348.2895.

Alternatively, ketal of compound 11 could be prepared via oxime formation from ketone 8. A solution of compound 8 (730 mg, 2.29 mmol, 1 equiv) and H₂NOH.HCl (264 mg, 3.8 mmol, 1.6 equiv) in pyridine (7.3 mL) was refluxed for 16 hours. Upon completion, the reaction mixture was filtered, washed with water and air dried to obtain the product (694 mg, 91%). A solution of the product in isopropanol (45 mL) was heated to reflux and Na metal (4.14 g, 180 mmol, 100 equiv) was added in portions over 4 hours. Water was added and the isopropanol was evaporated. The aqueous phase was extracted with CH₂Cl₂ (4×). The combined organic phases were dried (Na₂SO₄) and the solvent was evaporated to provide the product (yield: 90%). Subsequently, the crude product was dimethylated following the procedure (Method B) as described for compound 7 (yield: 82%).

### Preparation of Dimethylaminoketone (+)-11.

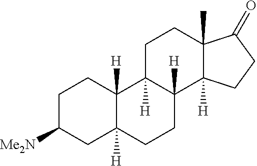

The crude product amine from the previous reaction (0.26 mmol) was dissolved in acetone (2.6 mL), and PTSA.H₂O (105 mg, 0.55 mmol, 2.1 equiv) was added. The reaction was stirred at room temperature for 30 minutes. The acetone was evaporated and residue was dissolved in water (5 mL). The aqueous phase was washed with Et₂O (2×2 mL). The aqueous phase was treated with 2 M NaOH solution and the pH was adjusted to 11-12. The aqueous phase was extracted with benzene (4×3 mL). The combined organic phases were dried (Na₂SO₄) and the solvent was evaporated. The residue was purified by flash chromatography (10% MeOH in CHCl₃ on SiO₂ that was pretreated with 1% Et₃N in CHCl₃) to afford the product (85 mg, 96% for three steps). ¹H NMR (500 MHz, CDCl₃) δ 2.42 (dd, J=19.23, 8.74 Hz, 1H), 2.28 (s, 6H), 2.25-2.11 (m, 1H), 2.10-2.00 (m, 1H), 1.98 (d, J=12.87 Hz, 1H), 1.96-1.87 (m, 2H), 1.83 (d, J=13.53 Hz, 1H), 1.80-1.70 (m, 3H), 1.65 (d, J=12.32 Hz, 1H), 1.49 (td, J=21.23, 10.53, 10.53 Hz, 1H), 1.33-1.18 (m, 5H), 1.17-1.04 (m, 2H), 0.96 (dd, J=41.78, 11.29 Hz, 2H), 0.85 (s, 3H), 0.84-0.76 (m, 1H), 0.75-0.53 (m, 2H); ¹³C NMR (125 MHz, CDCl₃) δ 221.6, 63.5, 50.8, 48.5, 48.1, 47.0, 42.2, 41.8, 40.9, 36.5, 36.0, 33.8, 31.7, 30.1, 29.4, 28.9, 25.3, 21.8, 14.0; IR (neat, cm⁻¹) 2916 (s), 2855 (m), 1737 (s), 1711 (w), 1694 (w), 1650 (w), 1555 (w), 1537 (w), 1468 (w), 1450 (w), 1333 (w); [α]_{D}²⁰=+36.1 (c 0.26, CHCl₃); HRMS (ESI-MS) calcd. for C₂₀H₃₄NO [(M+H)⁺] 304.2634, found 304.2648.

### Preparation of Dimethylaminotriflate of Ketone (+)-13.

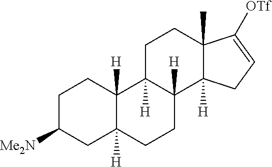

Ketone 11 (80 mg, 0.26 mmol, 1 equiv) was dissolved in THF (1 mL) and a 0.5 M solution of KHMDS (1.05 mL, 0.52 mmol, 2 equiv) in THF was added at 0° C. The reaction mixture was stirred for 30 minutes after which a 0.5 M solution of PhN(SO₂CF₃)₂ (0.78 mL, 0.39 mmol, 1.5 equiv) in THF was added. The reaction mixture was allowed to warm to room temperature and stirred for 10 h. Water (3 mL) was added and the phases were separated. The aqueous phase was extracted with Et₂O (2 mL) and EtOAc (2×2 mL), the combined organic phases were dried (Na₂SO₄) and the solvent was evaporated. The residue was purified by flash chromatography (2% MeOH in CHCl₃ on SiO₂ that was pretreated with 1% Et₃N in CHCl₃) to afford the product (98 mg, 87%). ¹H NMR (500 MHz, CDCl₃) δ 5.56 (dd, J=3.16, 1.64 Hz, 1H), 2.90-2.66 (m, 1H), 2.57 (s, 6H), 2.19 (ddd, J=15.02, 6.38, 3.50 Hz, 1H), 2.06-1.91 (m, 3H), 1.88-1.73 (m, 2H), 1.74-1.63 (m, 2H), 1.64-1.51 (m, 3H), 1.42 (dt, J=12.79, 12.77, 4.39 Hz, 1H), 1.23 (m, 3H), 1.17-1.07 (m, 1H), 1.02 (m, 2H), 0.96 (s, 3H), 0.88-0.75 (m, 1H), 0.75-0.57 (m, 2H); ¹³C NMR (125 MHz, CDCl₃) δ 159.4, 118.6 (q), 114.6, 64.3, 53.6, 48.3, 46.3, 45.1, 41.7, 40.2, 39.2, 34.4, 33.2, 32.8, 31.1, 29.7, 28.5, 26.9, 25.2, 15.4; IR (neat, cm⁻¹) 2910 (s), 2860 (m), 2768 (w), 1625 (w), 1454 (w), 1413 (w), 1208 (s), 1143 (s), 1067 (w), 1043 (w); [α]_{D}²⁰=+10.6 (c 0.63, CHCl₃); HRMS (ESI-MS) calcd. for C₂₁H₃₃F₃NO₃S [(M+H)⁺] 436.2127, found 436.2130.

### Preparation of (+)-EJC-02.

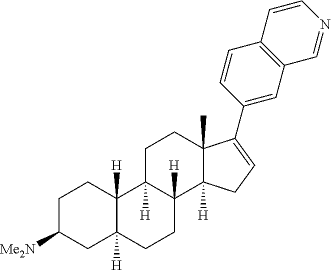

EJC-02 was prepared following the general procedure described for EJC-01. (Yield=68%). ¹H NMR (500 MHz, CDCl₃) δ 9.21 (s, 1H), 8.48 (d, J=4.47 Hz, 1H), 7.90 (s, 1H), 7.74 (s, 2H), 7.61 (d, J=5.45 Hz, 1H), 6.10 (s, 1H), 3.14-2.90 (m, 1H), 2.72 (s, 6H), 2.33-2.21 (m, 2H), 2.21-2.02 (m, 4H), 1.91-1.70 (m, 3H), 1.71-1.58 (m, 2H), 1.57-1.43 (m, 2H), 1.38 (dd, J=22.49, 10.22 Hz, 1H), 1.34-1.13 (m, 4H), 1.11 (s, 3H), 1.10-1.01 (m, 1H), 1.00-0.72 (m, 2H); ¹³C NMR (125 MHz, CDCl₃) δ 154.0, 152.2, 143.6, 141.7, 136.6, 135.6, 130.9, 129.6, 126.5, 124.2, 120.8, 65.0, 56.9, 48.1, 47.8, 46.3, 41.8, 39.8, 39.6, 35.6, 34.0, 33.3, 31.7, 30.7, 28.4, 26.6, 26.0, 16.9; IR (neat, cm⁻¹) 3027 (w), 2926 (s), 2865 (m), 1729 (w), 1674 (w), 1593 (w), 1466 (m), 1451 (m), 1411 (w), 1375 (w), 1254 (w), 1213 (w), 1092 (w); [α]_{D}²⁰=+18.8 (c 0.8, CHCl₃); HRMS (ESI-MS) calcd. for C₂₉H₃₉N₂ [(M+H)⁺] 415.3107, found 415.3120.

### Preparation of Compound (+)-EJC-07.

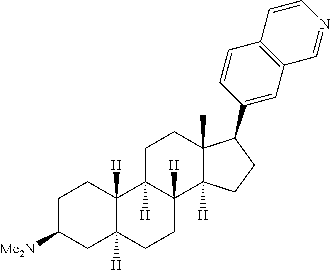

Compound EJC-02 (10 mg, 0.024 mmol) was dissolved in DMSO/THF (1:1, 480 μL) and KOOCN═NCOOK (80 mg, 0.48 mmol, 20 equiv) and AcOH (54 μL, 0.94 mmol, 40 equiv) was added at 0° C. in three portions over 2 hrs. The mixture was allowed to warm to room temperature and stirred for 24 hours. Brine was added and the mixture was extracted with Et₂O (5×1 mL). The combined organic layers were dried (Na₂SO₄) and the solvent was evaporated. The residue was purified on preparative TLC (500 μm, w/UV254; 5% MeOH in CHCl₃ containing 0.5% aqueous NH₃, then 2% MeOH in CHCl₃ containing 0.5% aqueous NH₃) to afford the product (13 mg, 65%). ¹H NMR (500 MHz, CDCl₃) δ 9.22 (s, 1H), 8.48 (d, 1H, J=5.3 Hz), 7.78 (s, 1H), 7.74 (d, 1H, J=8.4 Hz), 7.62 (d, 1H, J=5.5 Hz), 7.59 (d, 1H, J=8.5 Hz), 2.90 (t, 1H, J=9.7 Hz), 2.30 (s, 6H), 2.22 (m, 2H), 2.06 (m, 1H), 1.99 (m, 1H), 1.93 (d, 1H, J=12.6 Hz), 1.86 (m, 1H), 1.78 (m, 3H), 1.67 (dd, 1H, J=2.3 Hz, J=12.6 Hz), 1.60 (m, 1H), 1.43 (m, 1H), 1.35 (m, 3H), 1.13 (m, 3H), 1.02 (m, 2H), 0.95 (m, 1H), 0.86 (m, 1H), 0.69 (m, 2H), 0.51 (s, 3H); ¹³C NMR (125 MHz, CDCl₃) δ 152.5, 142.5, 141.0, 134.7, 132.6, 128.8, 126.2, 125.6, 120.3, 63.6, 57.5, 55.7, 48.5, 47.1, 45.1, 42.3, 42.0, 41.7, 38.1, 36.5, 34.1, 31.3, 29.9, 29.5, 29.0, 26.3, 25.8, 24.6, 13.1; IR (neat, cm⁻¹) 2919 (w), 2852 (s), 2770 (w), 1733 (w), 1683 (w), 1592 (w), 1448 (m), 1378 (m), 1335 (w), 1261 (w), 1208 (w), 1104 (w), 1033 (w); [α]_{D}²⁰=+13.8 (c 1, CHCl₃); HRMS (ESI-MS) calcd. for C₂₉H₄₁N₂ [(M+H)⁺] 417.3264, found 417.3258.

### Preparation of Compound (+)-EJC-09.

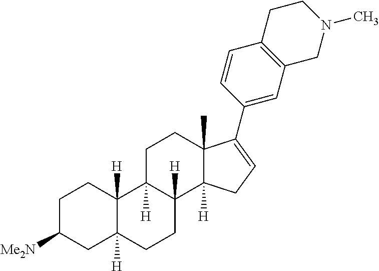

Compound EJC-02 (8 mg, 0.019 mmol) was dissolved in MeCN (250 μL) and 37% aqueous formaldehyde (170 μL, 120 equiv) was added followed by the addition of aqueous 30% AcOH solution (41 μL, 7.7 equiv). NaBH₃CN (6 mg, 0.095 mmol, 5 equiv) was added in three portions at room temperature over 1 h. The reaction was stirred for 4 hours. Upon completion, saturated aqueous NaHCO₃ was added. The aqueous phase was extracted with Et₂O (5 mL) and EtOAc (3×5 mL), the combined organic phases were dried (Na₂SO₄) and the solvent was evaporated to provide the crude product (74%). ¹H NMR (500 MHz, CDCl₃) 7.10 (d, 1H, J=7.5 Hz), 7.01 (m, 2H), 5.82 (s, 1H), 5.54 (s, 2H), 2.88 (t, 2H, J=5.7 Hz), 2.67 (m, 2H), 2.51 (s, 6H), 2.44 (s, 3H), 2.16 (ddd, 1H, J=3.4 Hz, J=6.6 Hz, J=15.5 Hz), 1.99 (m, 4H), 1.86 (d, 1H, J=8.4 Hz), 1.78 (d, 1H, J=11.2 Hz), 1.72 (dd, 1H, J=2.9 Hz, J=12.3 Hz), 1.65 (m, 2H), 1.54 (m, 1H), 1.41 (m, 1H), 1.26 (m, 3H), 1.07 (m, 3H), 0.99 (s, 3H), 0.85 (m, 2H), 0.71 (m, 2H); ¹³C NMR (125 MHz, CDCl₃) δ 154.9, 134.9, 134.5, 132.5, 129.0, 128.5, 124.8, 124.7, 64.2, 58.3, 56.9, 53.1, 48.3, 47.6, 46.7, 46.2, 42.0, 40.6, 39.9, 35.7, 35.0, 33.7, 31.4, 30.9, 29.9, 28.8, 27.7, 26.1, 16.9. IR (neat, cm⁻¹) 2923 (w), 2849 (s), 2770 (w), 1651 (m), 1488 (w), 1480 (w), 1375 (w), 1290 (m), 1256 (w), 1199 (w), 1156 (w), [α]_{D}²⁰=+7.8 (c 1, CHCl₃); HRMS (ESI-MS) calcd. for C₃₀H₄₅N₂ [(M+H)⁺] 433.3577, found 433.3579.

### Preparation of Compound (+)-EJC-08.

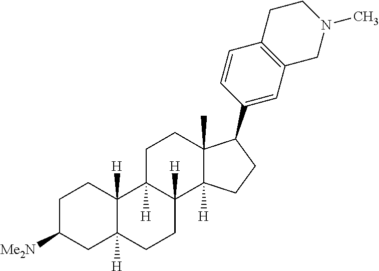

Compound EJC-08 was prepared from EJC-7 following the general procedure described for compound EJC-09. ¹H NMR (500 MHz) δ 7.01 (dd, 2H, J=7.7 Hz, J=18.5 Hz), 6.86 (s, 1H), 3.59 (s, 2H), 2.91 (m, 2H), 2.71 (m, 2H), 2.64 (s, 3H), 2.39 (s, 6H), 2.23 (m, 1H), 2.00 (m, 4H), 1.78 (m, 4H), 1.60 (m, 4H), 1.32 (m, 2H), 1.27 (s, 3H), 1.02 (m, 4H), 0.88 (m, 4H), 0.71 (m, 2H); ¹³C NMR (125 MHz, CDCl₃) δ 138.7, 133.8, 131.3, 128.5, 126.9, 126.7, 63.8, 58.2, 57.0, 55.4, 53.2, 48.8, 48.3, 46.9, 46.2, 43.8, 42.1, 41.9, 37.9, 35.8, 34.0, 32.1, 29.3, 28.3, 26.3, 25.8, 24.5, 22.9, 12.9; IR (neat, cm⁻¹) 2959 (s), 2920 (s), 2855 (w), 1731 (w) 1669 (w), 1507 (w), 1466 (w), 1259 (m), 1086 (m); 1256 (w), 1199 (w), 1156 (w), [α]_{D}²⁰=+6 (c 0.3, CHCl₃); HRMS (ESI-MS) calcd. for C₃₀H₄₇N₂ [(M+H)⁺] 435.3733, found 435.3728.

### Preparation of Isoquinoline Coupled Product (+)-EJC-10.

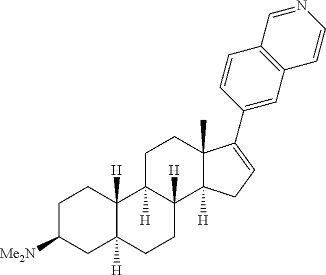

EJC-10 was prepared following the general procedure described for EJC-01. (Yield=53%). ¹H NMR (500 MHz, CDCl₃) δ 9.18-9.15 (s, 1H), 8.47 (d, J=5.51 Hz, 1H), 7.86 (d, J=8.56 Hz, 1H), 7.73 (s, 1H), 7.63 (d, J=8.50 Hz, 1H), 7.58 (d, J=5.71 Hz, 1H), 6.12 (s, 1H), 2.80 (m, 1H), 2.59 (s, 6H), 2.42 (dd, J=19.26, 8.81 Hz, 1H), 2.26 (ddd, J=15.89, 6.46, 3.30 Hz, 1H), 2.20-2.11 (m, 2H), 2.11-2.04 (m, 2H), 2.01 (m Hz, 1H), 1.87-1.67 (m, 3H), 1.63 (dt, J=11.52, 11.45, 6.53 Hz, 1H), 1.49 (dt, J=12.77, 12.60, 3.87 Hz, 1H), 1.45-1.12 (m, 5H), 1.09 (s, 3H), 0.96-0.66 (m, 3H); ¹³C NMR (125 MHz, CDCl₃) δ 154.3, 152.2, 143.4, 139.3, 136.1, 130.4, 127.7, 127.4, 127.2, 123.0, 120.7, 64.5, 56.9, 50.7, 48.2, 47.7, 46.5, 41.9, 40.2, 39.8, 35.6, 33.5, 31.7, 30.8, 28.6, 27.3, 26.0, 16.9; IR (neat, cm⁻¹) 2922 (s), 2855 (s), 1734 (m), 1635 (w), 1625 (w), 1558 (w), 1489 (w), 1199 (w); [α]_{D}²⁰=+26.9 (c 1.0, CHCl₃); HRMS (ESI-MS) calcd. for C₂₉H₃₉N₂ [(M+H)⁺] 415.3107, found 415.3106.

### Preparation of (+)-EJC-11.

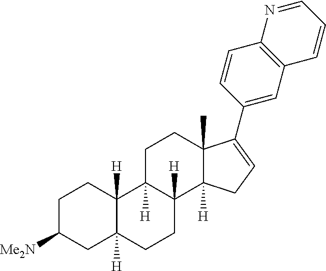

EJC-11 was prepared following the general procedure described for EJC-01. (Yield=60%). ¹H NMR (500 MHz, CDCl₃) δ 8.86 (dd, J=4.08, 1.35 Hz, 1H), 8.12 (d, J=7.83 Hz, 1H), 8.02 (d, J=8.62 Hz, 1H), 7.79 (d, J=1.74 Hz, 1H), 7.77 (s, 1H), 7.38 (dd, J=8.24, 4.21 Hz, 1H), 7.28 (s, 1H), 2.38 (s, 6H), 2.28 (ddd, J=15.75, 6.51, 3.30 Hz, 1H), 2.17 (ddd, J=12.34, 3.65, 2.44 Hz, 1H), 2.13-1.95 (m, 3H), 1.92-1.81 (m, 2H), 1.77 (dd, J=12.34, 2.99 Hz, 1H), 1.67 (m, 2H), 1.53 (dt, J=12.99, 12.82, 4.44 Hz, 1H), 1.38 (ddd, J=15.41, 12.31, 3.55 Hz, 1H), 1.34-1.14 (m, 3H), 1.12 (s, 3H), 1.10-0.99 (m, 3H), 0.94-0.81 (m, 2H), 0.82-0.70 (m, 2H); ¹³C NMR (125 MHz, CDCl₃) δ 154.4, 150.0, 147.7, 136.2, 135.7, 129.5, 129.2, 129.2, 128.4, 124.4, 121.4, 63.8, 57.1, 48.6, 47.9, 47.1, 42.3, 41.3, 40.0, 36.0, 35.8, 33.9, 31.7, 31.1, 29.2, 28.6, 26.2, 17.0; IR 2921, 2852, 1736, 1497, 1454, 1375, 1290, 1261, 1249, 1202, 1155, 1115, 1030 (neat, cm⁻¹); [α]_{D}²⁰=+33.5 (c 0.7, CHCl₃); HRMS (ESI-MS) calcd. for C₂₉H₃₉N₂ [(M+H)⁺] 415.3107, found 415.3104.

### Preparation of (+)-EJC-12.

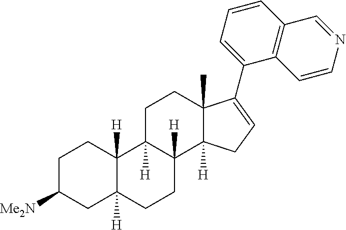

EJC-12 was prepared following the general procedure described for EJC-01. (Yield=64%). ¹H NMR (500 MHz, CDCl₃) δ 9.23 (s, 1H), 8.48 (s, 1H), 7.88 (d, J=8.12 Hz, 1H), 7.85 (d, J=5.69 Hz, 1H), 7.56 (dd, J=7.95, 7.31 Hz, 1H), 7.48 (dd, J=7.11, 0.93 Hz, 1H), 7.28 (s, 1H), 2.53 (s, 1H), 2.45 (s, 6H), 2.38 (s, 1H), 2.25-2.18 (m, 1H), 2.03 (dd, J=16.66, 7.48 Hz, 2H), 1.91 (d, J=8.72 Hz, 1H), 1.86-1.63 (m, 4H), 1.50 (dt, J=12.73, 12.63, 4.27 Hz, 1H), 1.45-1.34 (m, 1H), 1.33-1.02 (m, 7H), 0.97 (s, 3H), 0.94-0.73 (m, 3H); ¹³C NMR (125 MHz, CDCl₃) δ 152.8, 151.5, 143.0, 135.6, 135.3, 130.9, 129.6, 129.2, 129.2, 126.7, 126.5, 119.4, 64.1, 56.9, 50.0, 48.7, 46.9, 42.2, 40.9, 40.3, 35.3, 33.8, 32.3, 31.1, 29.0, 28.1, 26.0, 16.7; IR (neat, cm⁻¹) 2921 (s), 2851 (s), 1735 (w), 1460 (m), 1446 (m), 1377 (m), 1030; [α]_{D}²⁰=+21.2 (c 1.0, CHCl₃); HRMS (ESI-MS) calcd. for C₂₉H₃₉N₂ [(M+H)⁺] 415.3107, found 415.3110.

### Preparation of (+)-EJC-13.

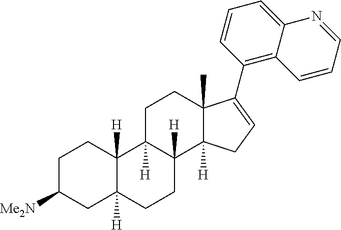

EJC-13 was prepared following the general procedure described for EJC-01. (Yield=60%). ¹H NMR (500 MHz, CDCl₃) δ 8.88 (d, J=3.79 Hz, 1H), 8.40 (d, J=8.49 Hz, 1H), 8.02 (d, J=8.46 Hz, 1H), 7.66 (t, J=7.79, 7.79 Hz, 1H), 7.39-7.29 (m, 2H), 5.75 (s, 1H), 2.38 (s, 6H), 2.19 (dd, J=14.98, 11.75 Hz, 1H), 2.05-1.91 (m, 2H), 1.89-1.66 (m, 6H), 1.47 (dt, J=12.58, 12.47, 4.34 Hz, 1H), 1.43-1.34 (m, 2H), 1.30-0.99 (m, 6H), 0.97 (s, 3H), 0.92-0.70 (m, 4H); ¹³C NMR (125 MHz, CDCl₃) δ 151.9, 150.2, 148.7, 136.5, 135.2, 130.8, 128.7, 128.4, 128.1, 126.0, 120.8, 63.9, 57.0, 50.0, 48.8, 47.0, 42.3, 41.2, 40.4, 35.9, 35.3, 33.9, 32.3, 31.2, 29.1, 28.4, 26.0, 16.6; IR (neat, cm⁻¹) 2921 (s), 2852 (s), 2771 (s), 1734 (m), 1570 (w), 1494 (w), 1456 (s), 1373 (w), 1261 (w), 1156 (w); [α]_{D}²⁰=+34.3 (c 0.6, CHCl₃); HRMS (ESI-MS) calcd. for C₂₉H₃₉N₂ [(M+H)⁺] 415.3107, found 415.3103.

### Preparation of (+)-EJC-14.

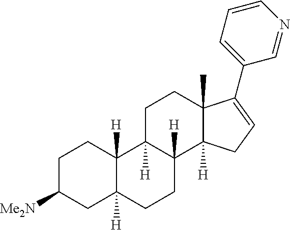

EJC-14 was prepared following the general procedure described for EJC-01. (Yield=64%). ¹H NMR (500 MHz, CDCl₃) δ 8.63 (s, 1H), 8.47 (d, J=3.53 Hz, 1H), 7.65 (dd, J=7.93, 1.73 Hz, 1H), 7.22 (dd, J=7.76, 4.79 Hz, 1H), 5.99 (s, 1H), 2.43 (s, 6H), 2.25 (ddd, J=15.76, 6.47, 3.25 Hz, 1H), 2.12-1.95 (m, 3H), 1.93-1.79 (m, 2H), 1.79-1.66 (m, 2H), 1.62 (dt, J=11.49, 11.46, 6.52 Hz, 1H), 1.47 (m, 1H), 1.41-1.19 (m, 5H), 1.21-1.04 (m, 4H), 1.02 (s, 3H), 0.96-0.81 (m, 2H), 0.82-0.70 (m, 1H); ¹³C NMR (125 MHz, CDCl₃) δ 152.0, 148.1, 148.0, 133.8, 129.3, 123.2, 110.9, 70.8, 64.0, 57.0, 48.5, 47.8, 46.9, 42.2, 41.1, 40.0, 35.7, 35.6, 33.8, 31.7, 31.0, 29.1, 28.3, 26.0, 16.8; IR (neat, cm⁻¹) 2928 (s), 2821 (s), 1733 (w), 1651 (m), 1556 (m), 1456 (m), 1372 (w), 1260 (w), 1104 (w), 1020; [α]_{D}²⁰=+12.0 (c 0.5, CHCl₃); HRMS (ESI-MS) calcd. for C₂₅H₃₇N₂ [(M+H)⁺] 365.2951, found 365.2943.

### Preparation of (+)-EJC-15.

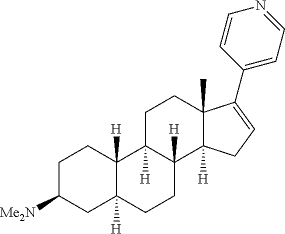

EJC-15 was prepared following the general procedure described for EJC-01. (Yield=71%). ¹H NMR (500 MHz, CDCl₃) δ 8.51 (d, J=4.79 Hz, 1H), 7.28 (d, J=0.97 Hz, 1H), 7.26 (d, J=5.19 Hz, 1H), 6.17 (s, 1H), 2.67 (m, 1H), 2.54 (s, 6H), 2.25 (ddd, J=16.18, 6.57, 3.34 Hz, 1H), 2.16-2.03 (m, 4H), 1.97 (d, J=10.10 Hz, 1H), 1.84 (m, 1H), 1.79-1.67 (m, 2H), 1.60 (dt, J=11.51, 11.46, 6.55 Hz, 1H), 1.45 (dt, J=12.71, 12.57, 4.09 Hz, 1H), 1.41-1.22 (m, 4H), 1.22-1.07 (m, 3H), 1.05 (s, 3H), 0.99-0.70 (m, 4H); ¹³C NMR (125 MHz, CDCl₃) δ 152.8, 149.9, 144.7, 131.5, 121.3, 64.3, 56.9, 48.3, 47.6, 46.7, 42.0, 40.6, 39.8, 35.4, 35.1, 33.6, 31.7, 30.9, 28.8, 27.7, 26.0, 16.9; IR (neat, cm⁻¹) 2958 (s), 2916 (s), 2831 (s), 1734 (s), 1585 (s), 1537 (m), 1478 (m), 1446 (m), 1413 (m), 1371 (m), 1104 (w); [α]_{D}²⁰=+7.0 (c 0.6, CHCl₃); HRMS (ESI-MS) calcd. for C₂₅H₃₇N₂ [(M+H)⁺] 365.29513, found 365.2943.

### Preparation of Compound (+)-17.

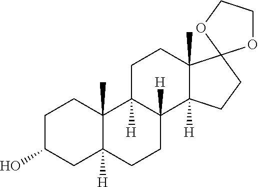

Compound 17 was prepared following the general procedure described for 9. (Yield=65%). The spectroscopic data of the compound were in agreement with the reported data.

### Preparation of Compound (+)-18.

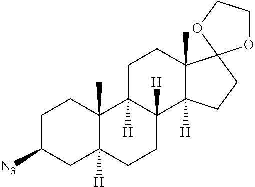

Compound 18 was prepared following the general procedure described for compound 10. (Yield=87%). ¹H NMR (500 MHz, CDCl₃) δ 4.03-3.74 (m, 4H), 3.27 (tt, J=11.91, 11.91, 4.65, 4.65 Hz, 1H), 1.98 (ddd, J=14.55, 11.67, 3.14 Hz, 1H), 1.87-1.74 (m, 3H), 1.74-1.62 (m, 3H), 1.62-1.43 (m, 3H), 1.38 (m, 4H), 1.34-1.18 (m, 3H), 1.13 (m, 1H), 0.99 (dt, J=13.78, 13.46, 3.81 Hz, 1H), 0.92 (ddd, J=24.39, 12.79, 5.19 Hz, 1H), 0.85 (s, 3H), 0.82 (s, 3H), 0.77 (dd, J=18.41, 10.06 Hz, 1H), 0.70 (ddd, J=13.85, 10.01, 3.85 Hz, 1H); ¹³C NMR (125 MHz, CDCl₃) δ 119.6, 65.4, 64.7, 60.7, 54.3, 50.5, 46.1, 45.4, 37.4, 35.9, 35.7, 34.4, 34.2, 31.4, 30.8, 28.6, 27.8, 22.8, 20.7, 14.6, 12.4; IR (neat, cm⁻¹) 2937 (s), 2856 (s), 2163 (m), 2083 (s), 1639 (br), 1454 (m), 1378 (w), 1303 (s), 1263 (m), 1208 (m), 1167 (s), 1102 (m), 1064 (w), 1036 (w), 963 (m); [α]_{D}²⁰=+6.8 (c 1, CHCl₃); HRMS (ESI-MS) calcd. for C₂₁H₃₄N₃O₂ [(M+H)⁺] 360.2645, found 360.2640.

### Preparation of Compound (+)-20.

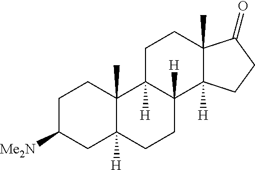

Compound 20 was prepared following the general procedure described for compound 11. (Yield=79% for three steps). ¹H NMR (500 MHz, CDCl₃) δ 2.44 (dd, J=19.31, 8.10 Hz, 1H), 2.30 (s, 6H), 2.17 (tt, J=11.71, 11.71, 3.97, 3.97 Hz, 1H), 2.07 (td, J=19.19, 9.08, 9.08 Hz, 1H), 1.97-1.89 (m, 1H), 1.85-1.70 (m, 4H), 1.70-1.64 (m, 1H), 1.61-1.44 (m, 3H), 1.43-1.18 (m, 7H), 1.11 (tt, J=12.25, 12.25, 3.30, 3.30 Hz, 1H), 1.04-0.89 (m, 2H), 0.87 (s, 3H), 0.81 (s, 3H), 0.70 (dt, J=12.00, 11.76, 3.90 Hz, 1H); ¹³C NMR (125 MHz, CDCl₃) δ 221.0, 64.0, 54.5, 51.5, 48.6, 48.1, 45.7, 41.4, 37.7, 36.1, 36.0, 35.2, 31.7, 31.0, 30.6, 28.7, 24.0, 21.9, 20.6, 14.0, 12.5; IR (neat, cm⁻¹) 2927 (s), 2855 (m), 2767 (w), 1740 (s), 1545 (m), 1376 (w), 1256 (w), 1198 (w), 1162 (w), 1118 (w), 1100 (w), 1061 (m), 1032 (m); [α]_{D}²⁰=+6.2 (c 1, CHCl₃); HRMS (ESI-MS) calcd. for C₂₁H₃₆NO [(M+H)⁺] 318.2791, found 318.2789.

Alternatively, the ketal of dimethylamine 20 could be prepared via oxime formation as described for compound 11.

### Preparation of Compound (+)-21.

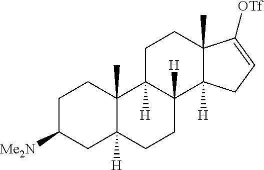

Ketone 20 (100 mg, 0.315 mmol, 1 equiv) was dissolved in THF (2.15 mL) and a 1 M solution of LHMDS (630 μL, 0.63 mmol, 2 equiv) in THF was added at 0° C. The reaction mixture was stirred for 15 minutes after which a 1 M solution of PhN(SO₂CF₃)₂ (472 μL, 0.47 mmol, 1.5 equiv) in THF was added. The reaction mixture was allowed to warm to room temperature and stirred for 1 h. Water (4 mL) was added and the phases were separated. The aqueous phase was extracted with Et₂O (2 mL) and EtOAc (2×2 mL), the combined organic phases were dried (Na₂SO₄) and the solvent was evaporated. The residue was purified by flash chromatography (2% MeOH in CHCl₃ on SiO₂ that was pretreated with 1% Et₃N in CHCl₃) to afford the product (128 mg, 74%). ¹H NMR (500 MHz, CDCl₃) δ 5.56 (s, 1H), 2.87 (ddd, J=12.28, 8.08, 3.42 Hz, 1H), 2.69 (s, 6H), 2.20 (ddd, J=14.82, 5.38, 3.46 Hz, 1H), 2.03-1.92 (m, 1H), 1.91-1.83 (m, 1H), 1.77 (td, J=13.43, 3.47, 3.47 Hz, 1H), 1.75-1.67 (m, 2H), 1.64-1.56 (m, 2H), 1.56-1.45 (m, 3H), 1.46-1.15 (m, 6H), 1.10 (m, 1H), 0.95 (s, 3H), 0.87-0.70 (m, 2H), 0.68 (s, 3H); ¹³C NMR (125 MHz, CDCl₃) δ 159.3, 117.2 (q), 114.7, 65.3, 54.4, 54.2, 45.5, 45.0, 40.2, 36.7, 35.8, 33.5, 32.7, 30.6, 29.0, 28.6, 22.4, 21.2, 20.6, 15.4, 12.1; IR (neat, cm⁻¹) 3031 (w), 2938 (s), 2858 (m), 1737 (w), 1627 (m), 1592 (m), 1489 (s), 1419 (s), 1297 (s), 1202 (s), 1144 (s), 1047 (m), 1006 (m); [α]_{D}²⁰=15.3 (c 1, CHCl₃); HRMS (ESI-MS) calcd. for C₂₂H₃₅F₃NO₃S [(M+H)⁺] 450.2284, found 450.2301.

### Preparation of (+)-EJC-16.

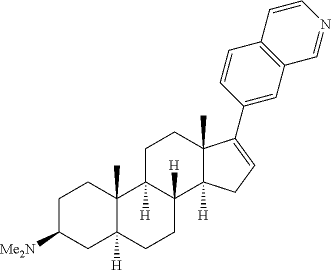

EJC-16 was prepared following the general procedure described for EJC-01. (Yield=53%). ¹H NMR (500 MHz, CDCl₃) δ 9.22 (s, 1H), 8.49 (s, 1H), 7.91 (s, 1H), 7.75 (s, 1H), 7.61 (d, J=5.28 Hz, 1H), 7.28 (d, J=1.38 Hz, 1H), 6.10 (s, 1H), 2.51 (s, 6H), 2.29 (ddd, J=15.63, 5.03, 2.98 Hz, 1H), 2.18 (d, J=7.62 Hz, 1H), 2.13-2.01 (m, 2H), 1.83 (ddd, J=26.77, 17.55, 8.76 Hz, 3H), 1.67 (m, 4H), 1.51 (m, 2H), 1.42-1.22 (m, 5H), 1.18 (m, 1H), 1.12 (s, 3H), 1.03 (m, 1H), 0.88 (s, 3H), 0.82 (t, J=11.14 Hz, 1H); ¹³C NMR (125 MHz, CDCl₃) δ 154.2, 152.8, 142.9, 142.9, 136.5, 134.9, 130.4, 129.4, 129.0, 126.4, 124.1, 64.8, 57.7, 54.7, 47.8, 45.9, 41.2, 41.0, 37.4, 36.1, 36.0, 35.8, 34.2, 32.0, 31.9, 28.9, 21.4, 17.0, 12.5; IR (neat, cm⁻¹) 2919 (s), 2848 (m), 1731 (m), 1590 (w), 1466 (m), 1446 (m), 1378 (w), 1296 (w); 1260 (m), 1196 (w), 1155 (m), 1102 (m), 1020 (w); [α]_{D}²⁰=+12.75 (c 0.8, CHCl₃); HRMS (ESI-MS) calcd. for C₃₀H₄₁N₂ [(M+H)⁺] 429.3264, found 429.3262.

### Preparation of (+)-EJC-17.

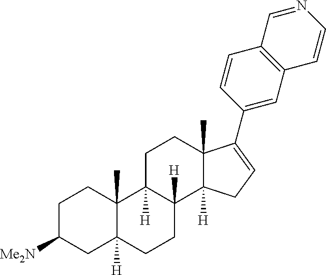

EJC-17 was prepared following the general procedure described for EJC-01. (Yield=73%). NMR (500 MHz, CDCl₃) δ 9.20 (s, 1H), 8.51 (s, 1H), 7.89 (d, J=8.57 Hz, 1H), 7.76 (s, 1H), 7.66 (dd, J=8.57, 1.51 Hz, 1H), 7.61 (d, J=5.65 Hz, 1H), 6.15 (dd, J=3.10, 1.75 Hz, 1H), 2.30 (ddd, J=15.76, 6.35, 3.33 Hz, 1H), 2.17 (dd, J=8.17, 2.34 Hz, 1H), 2.08 (m, 1H), 1.80 (ddd, J=11.69, 10.81, 5.82 Hz, 2H), 2.43 (s, 6H), 2.41 (s, 2H), 1.66 (m, 4H), 1.49 (dd, J=12.94, 5.03 Hz, 2H), 1.33 (m, 4H), 1.12 (s, 3H), 1.09-0.94 (m, 2H), 0.88 (s, 3H), 0.82 (m, 2H); ¹³C NMR (125 MHz, CDCl₃) δ 154.4, 152.2, 143.4, 139.5, 136.1, 130.5, 127.4, 127.3, 123.0, 120.8, 64.6, 57.8, 54.8, 47.9, 46.0, 41.3, 37.5, 36.1, 35.7, 34.2, 32.0, 32.0, 30.6, 29.0, 24.1, 21.3, 17.0, 14.0, 12.5; IR (neat, cm⁻¹) 2925 (s), 2584 (m), 2769 (w), 1736 (m), 1674 (w), 1626 (m), 1596 (w), 1488 (w), 1451 (m), 1406 (w), 1378 (m), 1261 (w), 1213 (w), 1148 (w), 1102 (w), 1034 (m), 830 (m), 751 (m), 687 (w); [α]_{D}²⁰=+10.12 (c 0.08, CHCl₃); HRMS (ESI-MS) calcd. for C₃₀H₄₁N₂ [(M+H)⁺] 429.3264, found 429.3262.

### Preparation of (+)-EJC-18.

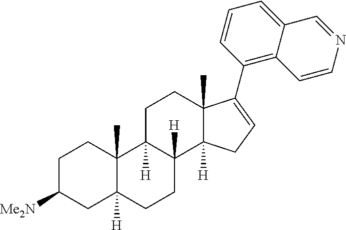

EJC-18 was prepared following the general procedure described for EJC-01. (Yield=64%). ¹H NMR (500 MHz, CDCl₃) δ 9.23 (s, 1H), 8.49 (d, J=5.73 Hz, 1H), 7.87 (dd, J=16.40, 6.97 Hz, 2H), 7.56 (t, J=7.63 Hz, 1H), 7.48 (d, J=7.14 Hz, 1H), 5.79 (m, 1H), 2.47 (s, 6H), 2.40 (ddd, J=15.36, 6.13, 3.06 Hz, 1H), 2.19 (dd, J=15.24, 11.18 Hz, 1H), 1.75 (m, 6H), 1.58 (m, 3H), 1.47 (m, 3H), 1.35 (m, 4H), 1.14 (m, 3H), 0.96 (s, 3H), 0.83 (s, 3H); ¹³C NMR (125 MHz, CDCl₃) δ 152.8, 151.4, 143.1, 135.6, 135.3, 130.9, 129.6, 126.7, 126.5, 119.4, 110.9, 64.8, 57.6, 55.0, 49.9, 46.0, 41.2, 37.4, 36.1, 35.3, 34.6, 32.4, 32.1, 30.4, 29.0, 23.9, 21.2, 16.7, 12.5; IR (neat, cm⁻¹) 2928 (s), 2852 (m), 2769 (w), 1735 (m), 1613 (w), 1579 (w), 1485 (m), 1452 (m), 1380 (m), 1298 (w), 1264 (m), 1200 (m), 1155 (m), 1097 (w), 1034 (m), 957 (w), 832 (m), 808 (w), 756 (s), 672 (m); [α]_{D}²⁰=+16.6 (c 1.8, CHCl₃); HRMS (ESI-MS) calcd. for C₃₀H₄₁N₂ [(M+H)⁺] 429.3264, found 429.3268.

### Preparation of (+)-EJC-19.

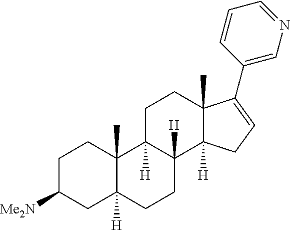

EJC-19 was prepared following the general procedure described for EJC-01. (Yield=60%). ¹H NMR (500 MHz, CDCl₃) δ 8.62 (s, 1H), 8.47 (d, J=4.39 Hz, 1H), 7.64 (td, J=7.86, 1.82 Hz, 1H), 7.22 (dd, J=7.84, 4.94 Hz, 1H), 5.98 (dd, J=3.07, 1.69 Hz, 1H), 2.46 (s, 6H), 2.25 (ddd, J=15.72, 6.36, 3.27 Hz, 2H), 2.03 (m, 4H), 1.79 (m, 4H), 1.63 (m, 4H), 1.39 (m, 4H), 1.17 (ddd, J=12.05, 10.08, 3.00 Hz, 1H), 1.02 (s, 3H), 0.86 (s, 3H), 0.80 (m, 2H); ¹³C NMR (125 MHz, CDCl₃) δ 152.0, 148.1, 148.0, 133.8, 129.3, 123.2, 64.7, 57.6, 54.7, 47.7, 46.0, 41.2, 37.4, 37.4, 36.1, 35.5, 34.2, 32.0, 31.9, 30.4, 28.9, 23.9, 21.3, 16.9, 12.5; IR (neat, cm⁻¹) 3036 (w), 2927 (s), 2852 (m), 2769 (w), 1736 (w), 1599 (w), 1473 (m), 1449 (m), 1409 (w), 1377 (m), 1296 (w), 1157 (m), 1024 (w), 926 (w), 798 (m), 755 (w), 708 (m); [α]_{D}²⁰=+13.4 (c 1.0, CHCl₃); HRMS (ESI-MS) calcd. for C₂₆H₃₉N₂ [(M+H)⁺] 379.3108, found 379.3113.

### Preparation of (+)-EJC-20.

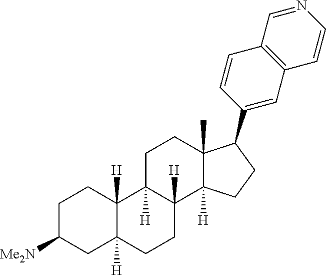

EJC-20 was prepared following the general procedure described for EJC-07. (Yield=63%). ¹H NMR (500 MHz, CDCl₃) δ 9.21 (s, 1H), 8.50 (d, J=4.03 Hz, 1H), 7.88 (d, J=8.49 Hz, 1H), 7.64 (s, 1H), 7.61 (d, J=5.62 Hz, 1H), 7.49 (d, J=8.87 Hz, 1H), 2.90 (t, J=9.65, 9.65 Hz, 1H), 2.65 (s, 1H), 2.52 (s, 6H), 2.25 (ddd, J=24.75, 11.30, 2.81 Hz, 1H), 2.14-1.99 (m, 2H), 1.98-1.90 (m, 1H), 1.90-1.81 (m, 1H), 1.81-1.73 (m, 1H), 1.72-1.67 (m, 1H), 1.65-1.55 (m, 1H), 1.35 (td, J=34.52, 11.43, 11.43 Hz, 5H), 1.21-0.96 (m, 5H), 0.96-0.77 (m, 3H), 0.79-0.66 (m, 2H), 0.51 (s, 3H); ¹³C NMR (125 MHz, CDCl₃) δ 152.2, 144.4, 143.1, 135.9, 129.5, 127.8, 126.8, 125.2, 120.5, 64.2, 57.7, 55.7, 48.2, 46.7, 45.3, 42.0, 41.8, 40.6, 38.0, 35.2, 33.8, 31.1, 29.0, 27.8, 26.3, 25.7, 24.5, 13.1; [α]_{D}²⁰=14.0 (c 0.5, CHCl₃) HRMS (ESI-MS) calcd. for C₂₉H₄₁N₂ [(M+H)⁺] 417.3264, found 417.3264.

### Preparation of (+)-S2.

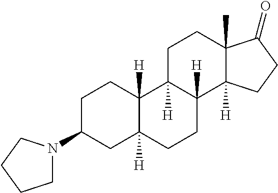

To a solution of S1 (120 mg, 0.37 mmol) in toluene (2.5 mL) was added 1,4-dibromobutane (53 μL, 97 mg, 0.45 mmol) and NaHCO₃ (63 mg, 0.75 mmol). The reaction mixture was refluxed under Dien-Stark conditions for 24 h. After cooling, the reaction mixture was filtered and concentrated. The crude product was dissolved in acetone (1.5 mL), and p-toluenesulfonic acid was added. The mixture was stirred for 20 minutes. After completion, the solvent was evaporated, water and the solution was washed with ether (2×1 mL). The aqueous phase was treated with 2 M NaOH solution and the pH was adjusted to 12. The aqueous phase was extracted with benzene (4×3 mL). The combined organic phases were dried (Na₂SO₄) and the solvent was evaporated to afford the product (101 mg, 82% for two steps). ¹H NMR (500 MHz, CDCl₃) □□δ 2.58 (m, 2H), 2.45 (m, 2H), 2.07 (m, 2H), 1.91 (m, 5H), 1.79 (m, 6H), 1.67 (m, 1H), 1.51 (m, 2H), 1.27 (m, 5H), 1.12 (m, 2H), 1.00 (m, 2H), 0.88 (s, 3H), 0.83 (m, 1H), 0.69 (m, 2H); ¹³C NMR (125 MHz, CDCl₃) □□221.8, 63.6, 52.4, 50.7, 48.5, 46.6, 41.7, 40.9, 40.1, 36.0, 33.7, 32.4, 31.8, 30.1, 29.1, 25.3, 23.8, 23.4, 21.8, 14.0, 8; IR (neat, cm⁻¹) 2920 (s), 2855 (m), 2779 (w), 1738 (s), 1451 (w), 1406 (w), 1374 (w), 1259 (w); [α]_{D}²⁰=+49.3 (c 1.00, CHCl₃); HRMS (ESI-MS) calcd. for C₂₂H₃₆NO [(M+H)⁺] 330.2791, found 330.2815.

### Preparation of Triflate of (+)-S2.

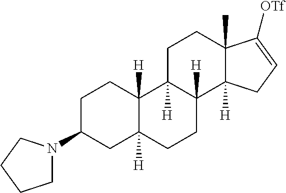

Triflate of S2 was prepared following the general procedure described for compound 13 (Yield=51%). NMR (500 MHz, CDCl₃) δ 5.57 (dd, 1H, J=1.5 Hz, J=3.1 Hz), 2.66 (bs, 4H), 2.48 (m, 1H), 2.21 (ddd, 1H, J=3.3 Hz, J=6.3 Hz, J=14.9 Hz), 2.07 (m, 1H), 1.96 (m, 3H), 1.84 (m, 5H), 1.69 (m, 4H), 1.60 (m, 1H), 1.43 (dt, 1H, J=4.4 Hz, J=12.7 Hz), 1.27 (m, 2H), 1.15 (m, 2H), 1.04 (m, 2H), 0.98 (s, 3H), 0.76 (m, 3H); ¹³C NMR (125 MHz, CDCl₃) δ 159.7, 120.2 (q), 114.5, 63.7, 53.9, 51.8, 48.8, 46.7, 45.2, 41.8, 39.5, 39.3, 33.5, 32.9, 30.0, 28.8, 28.6, 25.3, 23.8, 23.4, 15.5; IR (neat, cm⁻¹) 2925 (s), 2858 (m), 2778 (w), 1717 (w), 1628 (w), 1593 (w), 1489 (w), 1212 (s) 1142 (s); [α]_{D}²⁰=+20.7 (c 1.00, CHCl₃); HRMS (ESI-MS) calcd. for C₂₃H₃₅NO₃F₃S [(M+H)⁺] 462.2284, found 462.2295.

### Preparation of (+)-EJC-21.

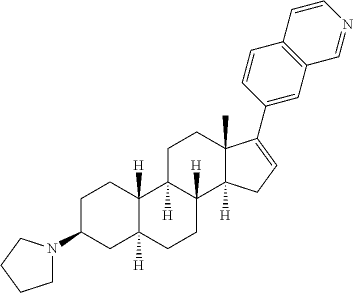

EJC-21 was prepared following the general procedure described for EJC-02. (Yield=55%). ¹H NMR (500 MHz, CDCl₃) δ 9.22 (s, 1H), 8.48 (d, 1H, J=5.4 Hz), 7.91 (s, 1H), 7.75 (s, 2H), 7.61 (d, 1H, J=5.6 Hz), 6.11 (dd, 1H, J=1.7 Hz, J=3.1 Hz), 3.17 (bs, 4H), 2.74 (m, 1H), 2.27 (ddd, 1H, J=3.3 Hz, J=6.5 Hz, J=15.8 Hz), 2.17 (m, 2H), 2.08 (m, 3H), 2.00 (dd, 1H, J=2.1 Hz, J=12.3 Hz), 1.85 (m, 1H), 1.78 (ddd, 1H, J=3.1 Hz, J=6.1 Hz, J=12.6 Hz), 1.66 (m, 3H), 1.52 (m, 3H), 1.33 (m, 5H), 1.12 (s, 3H), 1.07 (m, 1H), 0.91 (m, 3H), 0.73 (m, 1H); ¹³C NMR (125 MHz, CDCl₃) δ 154.2, 152.8, 142.9, 136.4, 134.9, 130.3, 129.3, 129.0, 126.4, 124.1, 120.3, 64.2, 57.0, 56.9, 51.2, 48.3, 47.9, 47.9, 46.0, 41.7, 39.7, 35.7, 33.4, 31.7, 30.9, 28.6, 26.0, 23.5, 17.0; IR (neat, cm⁻¹) 2923 (s), 2854 (m), 2675 (w), 2578 (w), 2481 (w), 1732 (w), 1592 (w), 1451 (w), 1374 (w), 1247 (w) 1149 (w); [α]_{D}²⁰=12.0 (c 0.5, CHCl₃) HRMS (ESI-MS) calcd. for C₃₁H₄₁N₂ [(M+H)⁺] 441.3264, found 441.3265.

### Preparation of (+)-EJC-22.

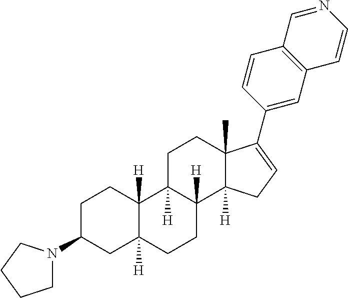

EJC-22 was prepared following the general procedure described for compound EJC-02. (Yield=53%). ¹H NMR (500 MHz, CDCl₃) δ 9.19 (s, 1H), 8.50 (s, 1H), 7.88 (d, 1H, J=8.5 Hz), 7.76 (s, 1H), 7.65 (dd, 1H, J=1.4 Hz, J=8.6 Hz), 7.60 (d, 1H, J=5.5 Hz), 6.14 (s, 1H), 3.16 (m, 4H), 2.75 (m, 1H), 2.27 (ddd, 1H, J=3.2 Hz, J=6.3 Hz, J=15.8 Hz), 2.08 (m, 7H), 1.63 (m, 8H), 1.31 (m, 4H), 1.12 (s, 3H), 1.06 (m, 1H), 0.89 (m, 3H), 0.73 (m, 1H); ¹³C NMR (125 MHz, CDCl₃) δ 154.4, 152.2, 143.5, 139.4, 136.2, 131.8, 130.4, 127.4, 127.2, 123.0, 120.8, 64.2, 57.0, 51.2, 48.3, 47.8, 46.0, 41.8, 39.8, 36.7, 35.6, 33.3, 31.8, 30.8, 29.7, 28.5, 26.0, 23.4, 17.0; IR (neat, cm⁻¹) 2925 (s), 2855 (m), 2669 (w), 2575 (w), 2483 (w), 2366 (w), 1734 (w), 1625 (w), 1487 (w), 1452 (w), 1376 (w), 1275 (w), 1159 (w); [α]_{D}²⁰=+13.0 (c 0.7, CHCl₃) HRMS (ESI-MS) calcd. for C₃₁H₄₁N₂ [(M+H)⁺] 441.3264, found 441.3266.

### Preparation of (+)-S3.

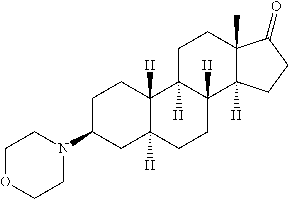

To a solution of S1 (100 mg, 0.31 mmol) in toluene (2 mL) was added 2-bromoethyl ether (52 μL, 96 mg, 0.42 mmol) and NaHCO₃ (55 mg, 0.65 mmol). The reaction mixture was refluxed under Dien-Stark conditions for 24 h. After cooling, the reaction mixture was filtered and concentrated. The crude product was dissolved in acetone (1.5 mL), and p-toluenesulfonic acid was added. The mixture was stirred for 20 minutes. After completion, the solvent was evaporated, water and the solution was washed with ether (2×1 mL). The aqueous phase was treated with 2 M NaOH solution and the pH was adjusted to 12. The aqueous phase was extracted with benzene (4×3 mL). The combined organic phases were dried (Na₂SO₄) and the solvent was evaporated to afford the product (90 mg, 85% for two steps). ¹H NMR (500 MHz, CDCl₃) δ 3.72 (m, 4H), 2.56 (m, 4H), 2.44 (dd, 1H, J=8.9 Hz, J=19.2 Hz) ppm 2.22 (tt, 1H, J=3.2 Hz, J=11.0 Hz) ppm 2.07 (td, 1H, J=9.1 Hz, J=19.3 Hz) ppm 1.96 (m, 2H), 1.82 (m, 4H), 1.68 (m, 1H), 1.51 (m, 1H), 1.27 (m, 3H), 1.13 (m, 3H), 0.99 (m, 3H), 0.88 (m, 3H), 0.82 (m, 2H), 0.68 (td, 2H, J=15.4 Hz, J=30.8 Hz); ¹³C NMR (125 MHz, CDCl₃) δ 221.6, 67.6, 63.5, 50.8, 50.1, 48.5, 48.1, 47.1, 42.2, 40.9, 36.6, 36.0, 33.8, 31.8, 30.1, 29.5, 28.9, 25.3, 21.8, 14.0; IR (neat, cm⁻¹) 2918 (s), 2852 (m), 1738 (s), 1449 (w), 1406 (w), 1266 (w), 1117 (m); [α]_{D}²⁰=+56.6 (c 1.00, CHCl₃); HRMS (ESI-MS) calcd. for C₂₂H₃₆NO₂ [(M+H)⁺] 346.2740, found 346.2763.

### Preparation of Triflate of (+)-S3.

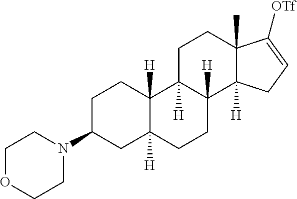

Triflate of S3 was prepared following the general procedure described for compound 13 (Yield=69%). ¹H NMR (500 MHz, CDCl₃) δ 5.56 (dd, 1H, J=1.6 Hz, J=3.1 Hz), 3.73 (m, 4H), 2.57 (s, 4H), 2.41 (m, 1H), 2.21 (m, 2H), 1.98 (m, 3H), 1.83 (m, 2H), 1.68 (m, 3H), 1.59 (dt, 1H, J=6.5 Hz, J=11.4 Hz), 1.43 (dt, 1H, J=4.5 Hz, J=12.8 Hz), 1.26 (m, 1H), 1.16 (m, 3H), 1.02 (m, 2H), 0.98 (s, 3H), 0.83 (m, 1H), 0.73 (m, 2H); ¹³C NMR (125 MHz, CDCl₃) δ 159.6, 118.7 (q), 114.6, 67.6, 63.5, 53.9, 50.0, 48.8, 47.1, 45.2, 42.3, 39.3, 36.5, 33.7, 33.0, 30.0, 29.2, 28.8, 28.6, 25.4, 15.5; IR (neat, cm⁻¹) 2921 (s), 2808 (m), 1738 (s), 1627 (w), 1449 (w), 1420 (m), 1212 (s) 1142 (m); [α]_{D}²⁰=+22.0 (c 1.00, CHCl₃); HRMS (ESI-MS) calcd. for C₂₃H₃₅F₃NO₃S [(M+H)⁺] 478.2233, found 478.2238.

### Preparation of (+)-EJC-23.

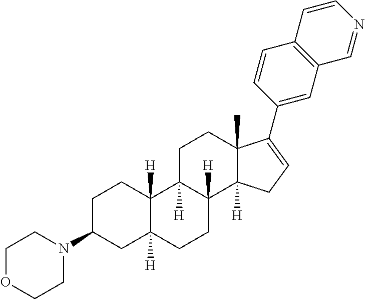

EJC-23 was prepared following the general procedure described for EJC-02. (Yield=55%). ¹H NMR (500 MHz, CDCl₃) δ 9.26 (m, 1H), 8.53 (s, 1H), 7.91 (s, 1H), 7.75 (s, 2H), 7.64 (s, 1H), 6.11 (s, 1H), 3.90 (s, 4H), 2.79 (s, 4H), 2.51 (s, 1H), 2.28 (ddd, 1H, J=3.3 Hz, J=6.4 Hz, J=15.8 Hz), 2.17 (d, 1H, J=12.2 Hz), 2.08 (m, 3H), 1.96 (s, 1H), 1.87 (d, 1H, J=11.2 Hz), 1.77 (dd, 1H, J=2.8 Hz, J=12.5 Hz), 1.70 (m, 1H), 1.65 (m, 1H), 1.53 (dt, 1H, J=3.7 Hz, J=12.6 Hz), 1.33 (m, 4H), 1.19 (m, 3H), 1.12 (s, 3H), 0.82 (m, 3H); ¹³C NMR (125 MHz, CDCl₃) δ 161.4, 158.6, 154.3, 152.7, 142.8, 136.6, 134.9, 130.3, 129.3, 126.4, 124.1, 66.3, 64.4, 57.1, 49.6, 48.5, 47.8, 46.9, 42.2, 39.9, 35.8, 35.7, 33.8, 31.7, 31.0, 29.0, 28.0, 26.1, 17.0; IR (neat, cm⁻¹) 2921 (s), 2851 (m), 2533.3 (w), 1733 (s), 1625 (w), 1594 (w), 1448 (m), 1267 (w), 1117 (s); [α]_{D}²⁰=25.6 (c 0.6, CHCl₃) HRMS (ESI-MS) calcd. for C₃₁H₄₁N₂O [(M+H)⁺] 457.3213, found 457.3214.

### Preparation of EJC-24.

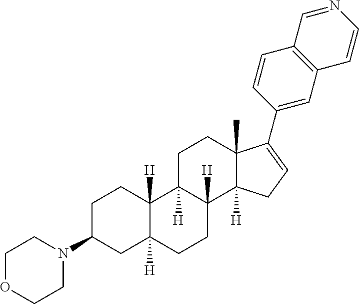

EJC-24 was prepared following the general procedure described for EJC-02. (Yield=44%). ¹H NMR (500 MHz, CDCl₃) δ 9.20 (m, 1H), 8.51 (s, 1H), 7.89 (d, 1H, J=8.6 Hz), 7.76 (s, 1H), 7.66 (dd, 1H, J=1.2 Hz, J=8.5 Hz), 7.61 (d, 1H, J=5.4 Hz), 6.15 (s, 1H), 3.94 (s, 4H), 2.85 (m, 5H), 2.29 (ddd, 1H, J=3.3 Hz, J=6.4 Hz, J=16.0 Hz), 2.13 (m, 5H), 1.86 (d, 1H, J=13.7 Hz), 1.76 (m, 2H), 1.66 (m, 1H), 1.58 (d, 1H, J=29.5 Hz), 1.51 (m, 1H) 1.30 (m, 5H), 1.13 (s, 3H), 1.06 (m, 1H), 0.90 (m, 2H), 0.76 (m, 1H); ¹³C NMR (125 MHz, CDCl₃) δ 154.4, 152.2, 143.4, 139.4, 136.1, 132.0, 130.4, 127.4, 127.3, 123.0, 120.8, 68.2, 65.0, 57.0, 49.3, 48.3, 47.9, 46.6, 42.1, 39.8, 35.7, 33.6, 31.7, 30.7, 29.5, 28.9, 27.1, 26.1, 17.0; [α]_{D}²⁰=+5.3 (c 0.6, CHCl₃) HRMS (ESI-MS) calcd. for C₃₁H₄₀N₂O [(M+H)⁺] 457.3213, found 457.3212.

### Preparation of 7-Bromoisoquinoline S6.

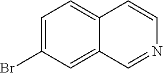

S6 was synthesized using a slightly modified procedure describing the preparation of 7-chloroisoquinoline; see Brown, E. V. J. Org. Chem. 1977, 42, 3208-3209, the teachings of which are incorporated herein by reference.

A 1 L round bottom flask, outfitted with Dean-Stark adapter, a reflux condenser, and a PTFE-coated stirbar, was charged with 3-bromobenzaldehyde (50.0 g, 270 mmol, 1 equiv), aminoacetaldehyde dimethyl acetal (28.42 g, 1 equiv) and benzene (270 mL). The resulting solution became cloudy within 2 minutes and was heated at reflux until approximately 5 mL of water is collected in the Dean-Stark adapter (˜7 hours). The solvent was then evaporated to afford the m-bromobenzalaminoacetal as a light yellow viscous liquid (˜75 g) which was directly used in the subsequent cyclization step.

In a 500 mL round bottom three-necked flask, outfitted with a mechanical stirrer and an addition funnel, phosphorous pentoxide (60.0 g) and concentrated sulfuric acid (15 mL) were mixed and stirred until a thick beige colored gum was formed. Next, m-bromobenzalaminoacetal (30 g, 110 mmol, 1 equiv) was dissolved in cold (5° C.) concentrated sulfuric acid (150 mL) and added slowly to the mixture of P₂O₅ and H₂SO₄ prepared above. The resulting dark colored reaction mixture was vigorously stirred and heated at 160° C. for 30 minutes. After cooling to room temperature, the dark brown viscous reaction mixture was carefully poured into ice water (1.5 L) while vigorously stirring. The pH was adjusted to 7 using 10N NaOH and the black tarry precipitate was filtered. The pH was then further increased to 9 using 10N NaOH. This basic aqueous phase was extracted with Et₂O (3×400 mL) and EtOAc (3×250 mL). The combined organic layers were washed with brine (3×200 mL), dried over MgSO₄ and evaporated to afford 11.5 g of brown oil. The crude product was then subjected to column chromatography (hexanes:EtOAc=3:1→2:1) to yield 8.5 g (37%) of pale yellow solid that is the 3:2 mixture of 7-bromoisoquinoline and 5-bromoisoquinoline, respectively.

### Separation of Regioisomers:

In a 1 L round bottom flask, equipped with a PTFE-coated stirbar, was charged with the mixture of 7-bromoisoquinoline and 5-bromoisoquinoline (8.4 g, 40 mmol), CH₂Cl₂ (400 mL), TMSBr (12.2 g, 80.6 mmol, 2 equiv) and MeOH (2.3 mL, 80.6 mmol, 2 equiv). The reaction mixture was stirred for 10 minutes at room temperature. Subsequently the solvent was evaporated yielding a crude mixture of isoquinoline HBr salts (11.5 g) as brownish yellow crystals. The crude HBr salt was dissolved in a minimum amount of EtOH (210 mL, 200 proof) at reflux. Next, Et₂O (10 mL) was added and reflux was continued until the solution became clear (˜10 min). The solution was then allowed to cool to room temperature over 12 h. The resulting small crystals were filtered to afford pure 7-bromoisoquinoline HBr salt (4.5 g). This HBr salt was dissolved in water (150 mL) and the pH was set to 10 using 2N NaOH. The aqueous phase was extracted with CHCl₃ (3×60 mL). The combined organic layers were washed with brine (2×50 mL), dried over Na₂SO₄ and evaporated to afford pure 7-bromoisoquinoline as a yellow solid (3.2 g). m.p.=78-79° C. ¹H NMR (500 MHz, CDCl₃) δ 9.19 (s, 1H), 8.56 (d, J=5.22 Hz, 1H), 8.14 (s, 1H), 7.77 (d, J=8.75 Hz, 1H), 7.71 (dd, J=8.71, 2.00 Hz, 1H), 7.63 (d, J=4.79 Hz, 1H); ¹³C NMR (125 MHz, CDCl₃) δ 151.6, 143.7, 134.4, 134.1, 129.9, 129.7, 128.4, 121.0, 120.4. IR (neat, cm⁻¹) 3017 (very weak), 1706 (s), 1627 (m), 1435 (m), 1356 (w), 1331 (m), 1295 (s), 1236 (m), 1156 (m), 1095 (m), 1032 (w), 966 (w); HRMS (ESI-MS) calcd. for C₉H₇BrN [(M+H)⁺] 207.97564, found 207.97588.

### Preparation of 7-Tributylstannyl Isoquinoline (S7).

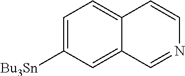

A 25 mL round bottom flask, equipped with a PTFE-coated stirbar, was charged with 7-bromoisoquinoline (300 mg, 1.44 mmol, 1 equiv) and THF (10 mL). The resulting solution was cooled to −78° C. and the 2.5 M solution of n-BuLi in THF (692 μL, 1.2 equiv) was added over 1 minute. The brownish-yellow reaction mixture was then stirred for 25 minutes. Next, Bu₃SnCl (587 μL, 1.5 equiv) was added via a syringe over 1 minute. Upon the addition of Bu₃SnCl, the brown color of the reaction mixture changed into light yellow. The reaction mixture was then allowed to warm to room temperature (1.5 h) and poured into a mixture of Et₂O/deionized water (50 mL/50 mL). The aqueous phase was extracted with Et₂O (2×25 mL) and the combined organic layers were washed with brine, dried over MgSO₄ and evaporated. The crude product (˜800 mg) was subjected to column chromatography (CHCl₃:EtOAc=15:1) to afford the product (510 mg, 85%) as a colorless viscous liquid. ¹H NMR (500 MHz, CDCl₃) δ 9.23 (s, 1H), 8.50 (d, J=5.70 Hz, 1H), 8.07 (s, 1H), 7.82-7.72 (m, 2H), 7.61 (d, J=5.72 Hz, 1H), 1.63-1.49 (m, 6H), 1.42-1.29 (m, 6H), 1.16 (m, 6H), 0.90 (t, J=7.33, 7.33 Hz, 9H); ¹³C NMR (125 MHz, CDCl₃) δ 152.5, 143.0, 142.3, 137.7, 136.3, 135.7, 128.6, 125.4, 120.5, 29.3, 27.5, 13.8, 9.9; IR (neat, cm⁻¹) 3048 (m), 2922 (br, s), 2636 (m), 2362 (w), 1926 (w), 1732 (w), 1699 (w), 1619 (s), 1576 (s), 1463 (s), 1417 (m), 1376 (s), 1337 (m), 1273 (m), 1145 (m), 1074 (s), 1031 (s), 960 (m); HRMS (ESI-MS) calcd. for C₂₁H₃₄NSn [(M+H)⁺] 420.17077, found 420.17090.

### Preparation of 6-Tributylstannyl Quinoline S9.

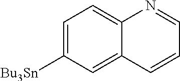

6-tributylstannyl quinoline was prepared as described for 7-tributylstannyl quinoline (Yield=70%). ¹H NMR (500 MHz, CDCl₃) δ 8.87 (dd, 1H, J=1.7 Hz, J=4.2 Hz), 8.10 (d, 1H, J=8.3 Hz), 8.06 (d, 1H, J=8.2 Hz), 7.91 (s, 1H), 7.81 (dd, 1H, J=1.0 Hz, J=8.2 Hz), 7.35 (dd, 1H, J=4.2 Hz, J=8.2 Hz), 1.58 (m, 6H), 1.35 (m, 6H), 1.15 (m, 6H), 0.89 (t, 9H, J=7.3 Hz); ¹³C NMR (125 MHz, CDCl₃) δ 150.4, 148.5, 141.3, 136.9, 136.4, 135.8, 128.4, 128.3, 121.1, 29.3, 27.5, 13.8, 9.9; IR (neat, cm⁻¹) 3048 (w), 2927 (s), 2852 (m), (w), 1608 (w), 1564 (w), 1488 (w), 1463 (w), 1417 (w), 1376 (w), 1339 (w), 1291 (w), 1148 (w), 1058 (w), 1031 (w), 960 (w); HRMS (ESI-MS) calcd. for C₂₁H₃₄NSn [(M+H)⁺] 420.1707, found 420.1718.

### Preparation of 5-Tributylstannyl Quinoline S10.

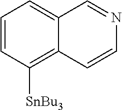

5-tributylstannyl quinoline was prepared as described for 7-tributylstannyl quinoline (Yield=96%). ¹H NMR (500 MHz, CDCl₃) δ 8.91 (dd, 1H, J=1.5 Hz, J=4.2 Hz), 8.08 (m, 2H), 7.68 (m, 2H), 7.40 (dd, 1H, J=4.2 Hz, J=8.4 Hz), 1.54 (m, 6H), 1.33 (m, 6H), 1.21 (m, 6H), 0.86 (t, 9H, J=7.3 Hz); ¹³C NMR (125 MHz, CDCl₃) δ 149.9, 149.1, 144.0, 138.2, 135.9, 134.0, 130.0, 129.0, 120.8, 29.3, 27.5, 13.8, 10.7; IR (neat, cm⁻¹) 3065 (w), 2956 (s), 2870 (s), 2852 (s), 1942 (w), 1880 (w), 1554 (m), 1489 (s), 1463 (s), 1418 (w), 1376 (w), 1376 (w), 1303 (w), 1123 (w), 1072 (w); HRMS (ESI-MS) calcd. for C₂₁H₃₄NSn [(M+H)⁺] 420.1707, found 420.1738.

### Preparation of 5-Tributylstannyl Isoquinoline S11.

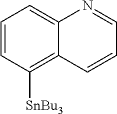

5-tributylstannylisoquinoline was prepared as described for 7-tributylstannyl quinoline (Yield=88%). ¹H NMR (500 MHz, CDCl₃) δ 9.23 (s, 1H), 8.54 (d, 1H, J=5.8 Hz), 7.91 (d, 1H, J=8.1 Hz), 7.83 (dd, 1H, J=1.3 Hz, J=6.7 Hz), 7.55 (m, 2H), 1.55 (m, 6H), 1.34 (m, 6H), 1.22 (m, 6H), 0.87 (t, 9H, J=7.3 Hz); ¹³C NMR (125 MHz, CDCl₃) δ 153.7, 143.1, 142.2, 141.7, 139.6, 129.1, 128.0, 126.9, 122.9, 29.3, 27.5, 13.8, 10.6; IR (neat, cm⁻¹) 3055 (w), 2959 (s), 2925 (s), 2870 (s), 2852 (w), 1613 (m), 1575 (w), 1463 (m), 1373 (m), 1259 (w); HRMS (ESI-MS) calcd. for C₂₁H₃₄NSn [(M+H)⁺] 420.1707, found 420.1709.

### Methods for In Vitro and In Vivo Functional Experiments

The compounds were tested in a double blind manner using in vitro human umbilical vascular endothelial (HUVE) cell growth, migration, and angiogenesis assays known to those of skill in the art.

### Compound Toxicity in HUVE Cells

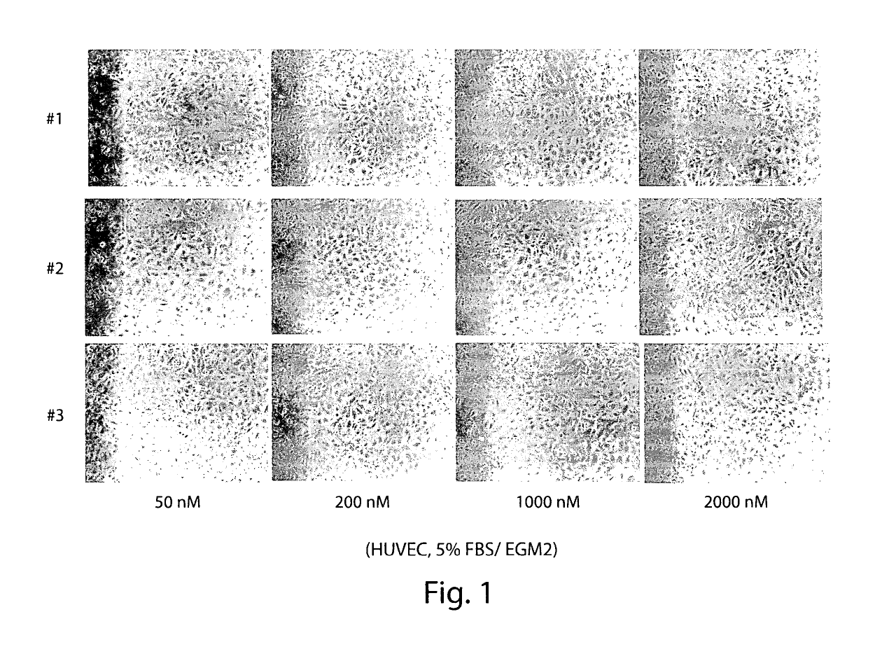

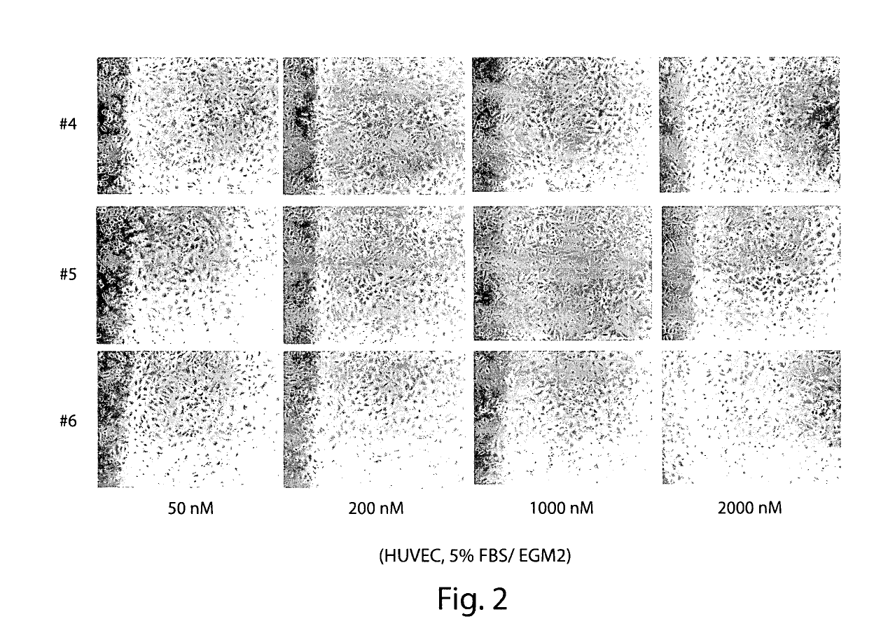

HUVE cells were cultured for 24 hours on standard tissue culture dishes in the presence of EJC 1-6 over a range of concentration (50-2000 nM). None of the six sets of HUVE cells showed any morphological change after 24 hours, suggesting that these compounds do not produce cell toxicity. (FIGS. 1 and 2).

### HUVE Cell Growth

Incorporation of 5-bromo-2′-deoxyuridibe (BrdU) is a common method used to detect the DNA replication in actively proliferating cells. Inhibition of cell growth is inferred by the decrease of BrdU incorporation into the cells. The effect of EJC-1, EJC-2, EJC-10, EJC-14 and EJC-16-20 on the growth of HUVE cells was measured by determining the percentage of cells that exhibited nuclear incorporation of BrdU into DNA, as detected by a procedure known in the art. The assay can be preformed in the presence or absence of growth factors such as VEGF, bFGF and PDGF. This assay was performed by a procedure described in Huang S, Chen C S, Ingber D E. “Control of cyclin D1, p 27(Kip1), and cell cycle progression in human capillary endothelial cells by cell shape and cytoskeletal tension.” Mol Biol Cell. 1998, 9:3179-93, the teachings of which are incorporated herein by reference.

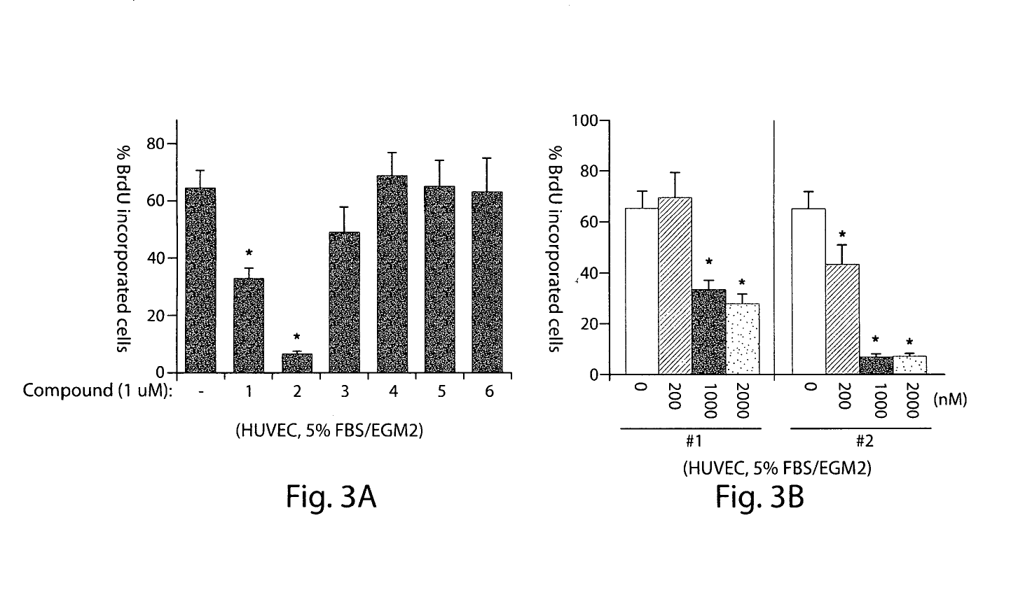

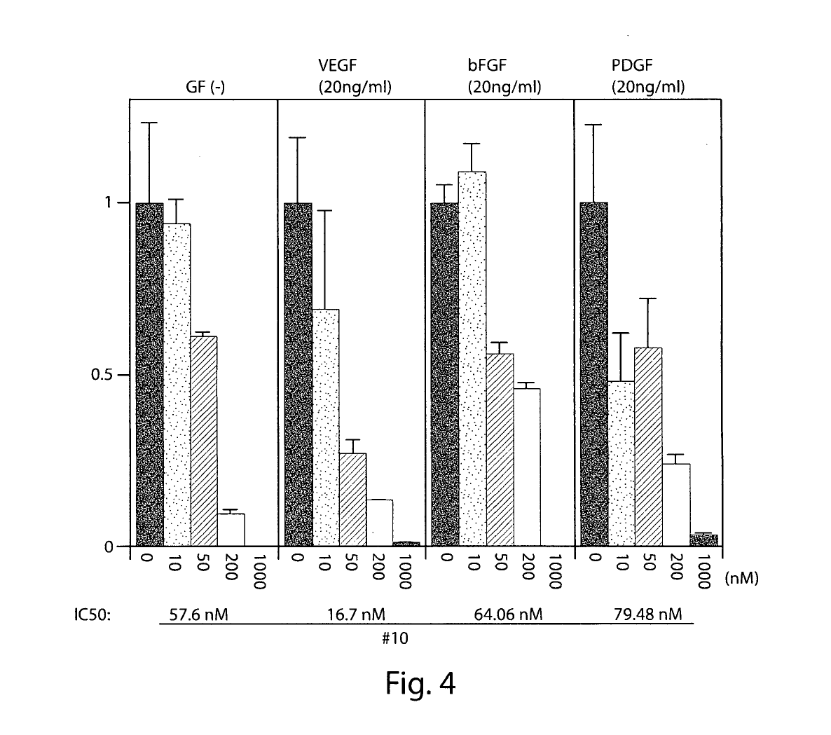

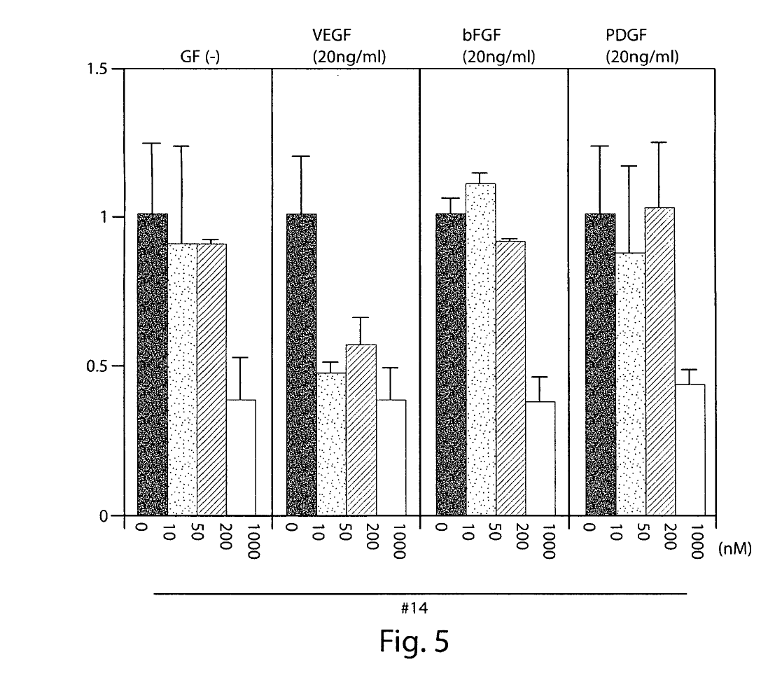

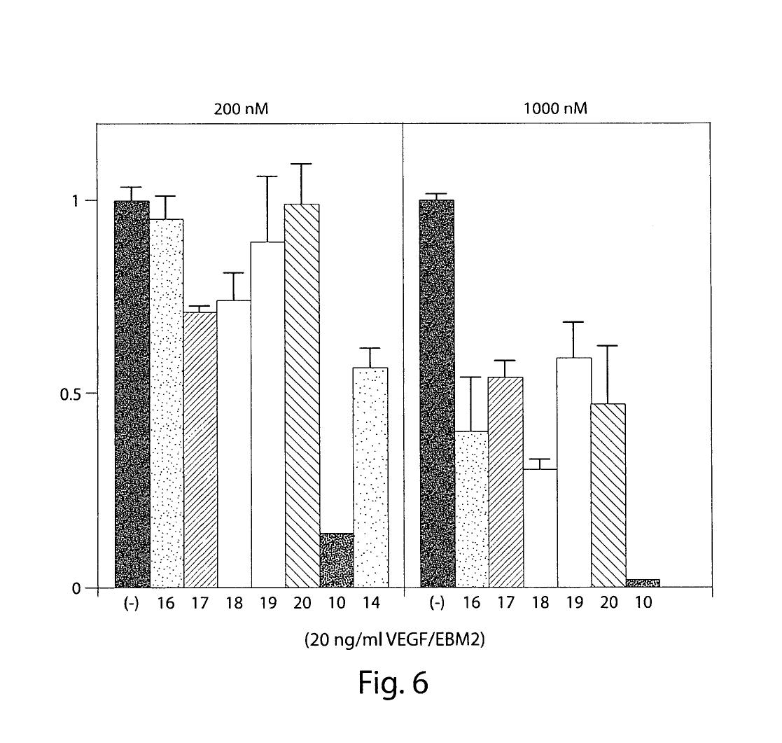

EJC-1 and EJC-2 inhibited the BrdU incorporation at doses greater than 1 μM and 200 nM, respectively (FIGS. 3A & 3B). EJC-10 inhibited cell growth induced by VEGF, bFGF and PDGF. The IC₅₀ of EJC-10 for cell growth induced by VEGF, bFGF and PDGF was 16.7, 64.1, and 79.5 nM, respectively, when tested individually against each of these growth factors (FIG. 4). EJC-14 inhibited cell growth induced by VEGF at 50 nM concentration, but inhibited growth induced by bFGF and PDGF only at higher concentrations (FIG. 5). EJC-16-20 did not inhibit cell growth induced by VEGF (20 ng/ml) at 200 nM. EJC-16-20 inhibited cell growth by half at 1000 nM (FIG. 6).

### Inhibition of HUVE Cell Migration Using Transwell Migration Assay

Cell migration is an important aspect of angiogenesis. The effect of the test compounds on HUVE cell motility as stimulated by VEGF was examined using a transwell migration assay. This assay was performed by a similar procedure described in Shimizu A, Mammoto A, Italiano J E Jr, Pravda E, Dudley A C, Ingber D E, Klagsbrun M. “ABL2/ARG tyrosine kinase mediates SEMA3F-induced RhoA inactivation and cytoskeleton collapse in human glioma cells.” J. Biol. Chem. 2008, 283:27230-8, the teaching of which are incorporated by reference.

Briefly, transwell membranes (Coster, N.Y.) were coated with 0.5% gelatin, and cells were seeded (10⁵ cells/100 μl) with 0.3% FBS/EBM2. The test compound was added to the both sides of the chamber. Cells were stained with Giemsa solution 16 hours later, and counted in 10 random fields (×400).

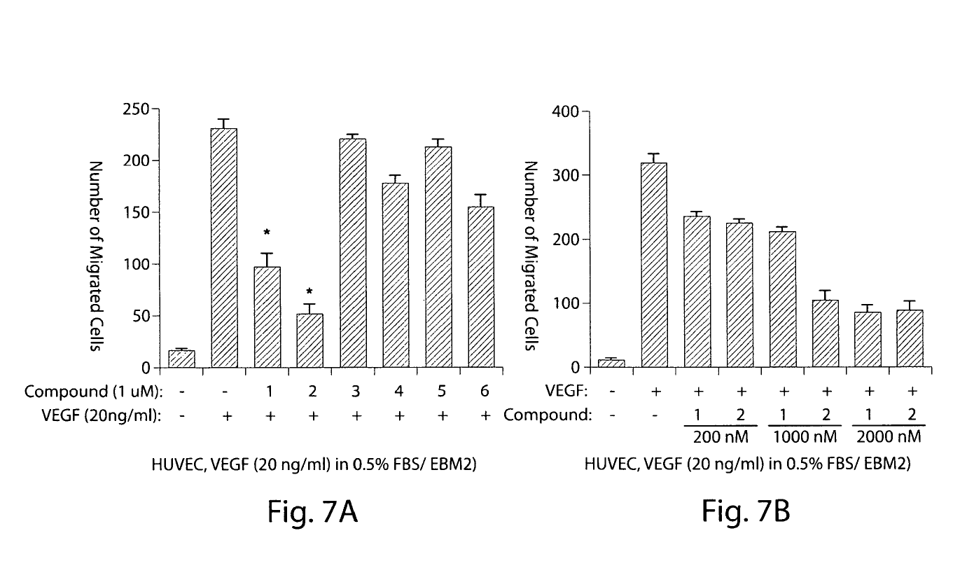

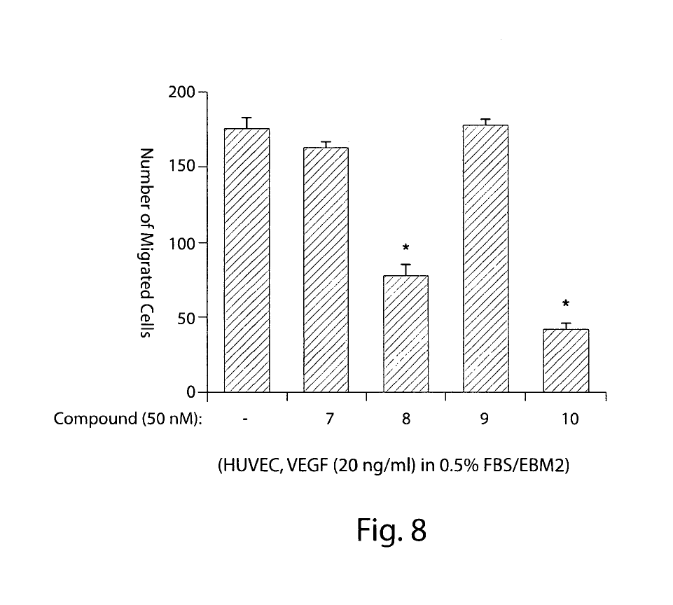

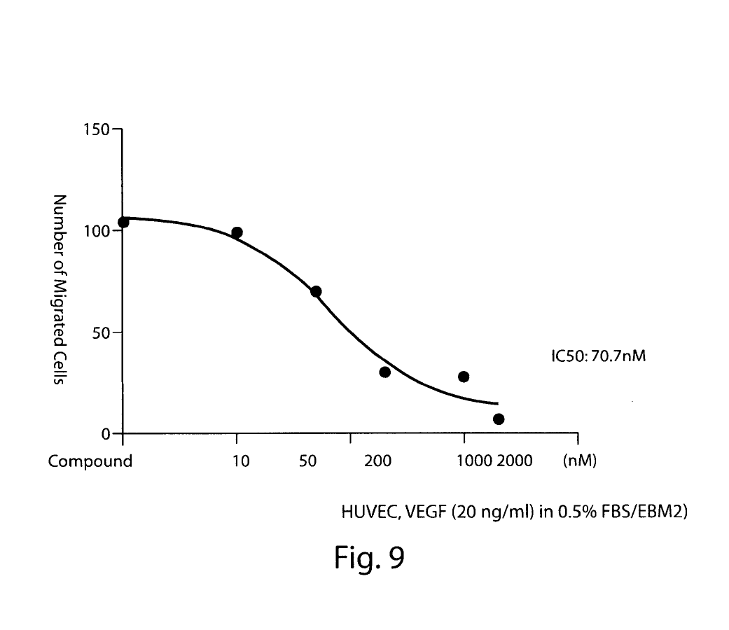

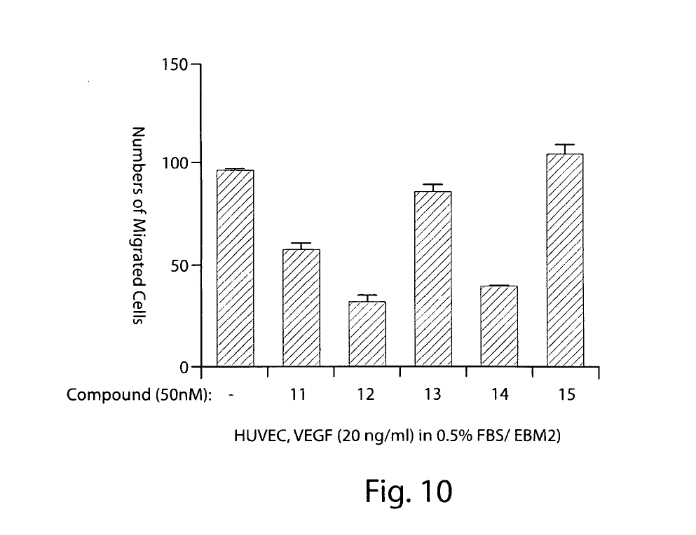

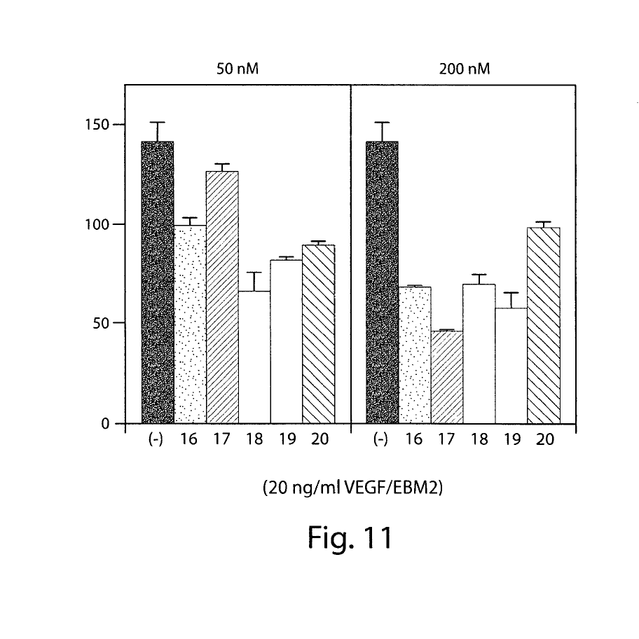

The effect of EJC-1, EJC-2 and EJC-7-20 on the migration of HUVE cells stimulated by VEGF using transwell migration assay was examined. EJC-1 and EJC-2 inhibited the HUVE cell migration at 1 μM. (FIGS. 7A and 7B). EJC-8 and 10 inhibited the HUVE cell migration at 50 nM (FIG. 8). The IC 50 of EJC-10 was 70.7 nM (FIG. 9). EJC-12 and EJC-14 inhibited the HUVE cell migration at 50 nM (FIG. 10). EJC-16-20 inhibited cell migration at 200 nM (FIG. 11). These cell migration assays revealed that EJC-10, EJC-12 and EJC-14 exhibit inhibitory activity at a concentration of 50 nM while EJC-11 and EJC-13 were moderately active at this concentration and EJC-15 was inactive. EJC-01 and EJC-02 exhibited inhibitory activity at 200 nM concentration. EJC-07 was active at 200 nM. EJC-09 was not active at 50 or 200 nM concentration. Pyrrolidine and morpholine derivatives (EJC-21, EJC-22, EJC-23 and EJC-24) did not inhibit cell migration at 50 nM concentration.

### Inhibition of Tube Formation

The effect of an agent on tube formation of HUVE cells was determined by a procedure described in Grant D S, Kinsella J L, Kibbey M C, LaFlamme S, Burbelo P D, Goldstein A L, Kleinman H K “Matrigel induces thymosin beta 4 gene in differentiating endothelial cells.” J Cell Sci. 1995, 108:3685-94, the teachings of which are incorporated by reference.

Briefly, HUVE cells (10⁴ cells/150 μl of EBM-2) were plated on Matrigel™ (BD biosciences) and incubated for 12-16 hrs in the presence of VEGF (20 ng/ml) and the test compound. Tube formation was assessed in 10 random fields (4×).

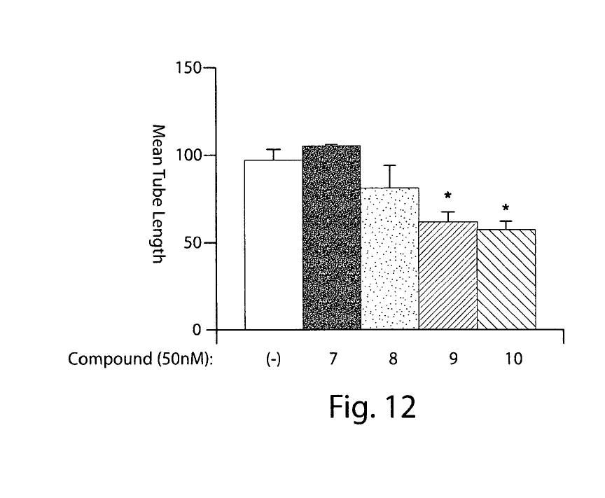

EJC-1-10 did not inhibit the tube formation in the 5% FBS/EGM2 media which includes growth factors (e.g., bFGF, VEGF, EGF, PDGF) at a concentration of 200 nM. EJC-2 inhibited tube formation when cultured with VEGF in the basal EBM2 medium at 200 nM. EJC-1 and EJC-2 inhibited the tube formation at 2 μM. EJC-9 and EJC-10 produced significant inhibition of tube formation when cultured with VEGF in the basal EBM2 medium at 50 nM. The mean tube length formed was measured in the micrographs taken of the HUVE cultures. Quantitative analysis of the mean tube length formed confirmed that EJC-9 and EJC-10 at 50 nM inhibited the tube formation when cultured with VEGF in the basal EBM2 medium (FIG. 12).

### In Vivo Screening Assay for Inhibitors of Retinal Angiogenesis

Blood vessel formation is critical for organ formation in the body. Compounds that inhibit angiogenesis within specific tissue types have therapeutic potential. For example, compounds that are effective in the retina may be used for the treatment of many types of blindness and other opthalmological diseases. Therefore, an in vivo method to screen for compounds that inhibit the growing vascular networks within the developing whole retina in eyes of living newborn mice was developed.

The developing vasculature of the newborn mouse retina has been shown to be an excellent model for analysis of the molecular and genetic mechanism of capillary development because the vascular network pattern is easily evaluated by lectin-staining and microscopic analysis. To screen in vivo in the eye for inhibitors of retinal angiogenesis, we adapted and modified the system described in Mammoto, A., Connor, K. M., Mammoto, T., Yung, C. W., Huh, D., Aderman, C. M., Mostoslaysky, G., Smith, L. E. H., & Ingber, D. E. A mechanosensitive transcriptional mechanism that controls angiogenesis. Nature 457, 1103-1108 (2009); and Shih, S. C., Ju, M., Liu, N. & Smith, L. E. Selective stimulation of VEGFR-1 prevents oxygen-induced retinal vascular degeneration in retinopathy of prematurity. J. Clin. Invest. 112, 50-7 (2003), the teachings of both are incorporated herein by reference. This assay was performed by a procedure described in Chen J, Connor K M, Aderman C M, Smith L E. “Erythropoietin deficiency decreases vascular stability in mice.” J. Clin. Invest. 2008, 118:526-33, the teachings of which are incorporated herein by reference.

Briefly, compounds that were found to have angiogenesis inhibitory activities in the in vitro models with cultured endothelial cells were injected at different doses (500 pmol-10 nmol/0.3-0.5 μl) into the eye in neonatal mice at 6 or 14 days post birth (P6 and P14, respectively).

After intraperitoneal injection of Avertin (125-240 mg/kg), the eyelid of mice was opened in a gentle manner by using tiny sterile blunt forceps. Test compound was injected into the vitreous of one eye of the mouse and a vehicle was injected into the other eye of the same mouse at either P6 or P14. Injections were performed by inserting an Exmire microsyringe (MS-NE05, ITO Corp. Fuji, Japan) into the vitreous 1 mm posterior to the corneal limbus. Pupils were dilated with 1% tropicamide. Insertion and the injection (0.3-0.5 μL solution) were directly viewed through an operating microscope, taking care not to injure the lens or the retina. The time points for intravitreous injection were set at P6 and P14 because these stages represent distinct developmental stages during vascular network formation. Blood vessels start spreading over the surface of retinal tissue at early times (P4-P6), then these distributed blood vessels start migrating into the deeper layer of the retina to make a three-dimensional vascular network at later times (P6-P14). Thus, using this approach, screening for inhibitors of initiation of angiogenesis at early times, and agents that induce regression of preexisting capillary vessels at the later time point can be identified.

After injection, the eyelid was treated with bacitracin eye antibiotic ointment, and eyelids naturally reformed after the procedure. Vascular network formation in the retina was assessed two days after injection using flat-mounted, fluorescein-conjugated isolectin-staining and immunohistochemical analysis. Quantification of vessel density was performed with Adobe photoshop.

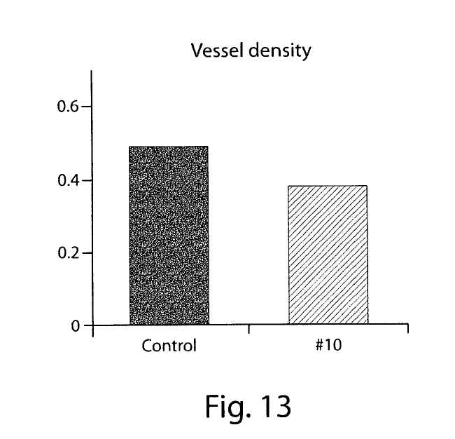

EJC-2 appeared to inhibit retinal angiogenesis based on morphological analysis, but the sampling numbers were low and significant effects were not observed at this dose (5-10 nmol in a single injection). A single injection of 500 pmol of EJC-10 inhibited retinal angiogenesis in p6 mice. A single injection of 5 nmol of EJC-10 did not inhibit retinal vessel formation completely in either p6 or p14 mice. Quantitative analysis of micrographs taken of the eye of p6 mice treated with EJC-10 indicated a decrease in the vessel density as compared to the control (FIG. 13). EJC-14 did not inhibit retinal vascular formation either in p6 or p14 mice (5 nmol).

While this invention has been particularly shown and described with references to example embodiments thereof, it will be understood by those skilled in the art that various changes in form and details may be made therein without departing from the scope of the invention encompassed by the appended claims.

1. A compound represented by the following structural formula: 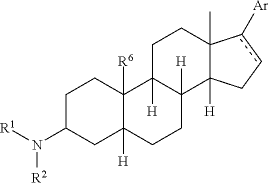
wherein
Ar is a heterocyclyl or heteroaryl, wherein each can be monocyclic or bicyclic and wherein each is optionally substituted by one to three groups represented by R³, or a phenyl or cycloalkyl, wherein the phenyl and cycloalkyl represented by Ar are substituted with —[(CH₂)₀₋₆]—N(R⁴)₂ and optionally substituted by one or two groups represented by R³; [symbol]
is a single or double bond;
R¹ and R² are each independently (a) hydrogen; or (b) (C₁-C₁₀)alkyl, (C₂-C₁₀)alkenyl, (C₂-C₁₀)alkynyl, aryl(C₀-C₃)alkyl, heteroaryl(C₀-C₃)alkyl, cycloalkyl(C₀-C₃)alkyl, heterocyclyl(C₀-C₃)alkyl, heteroaryl(C₀-C₃)alkyl, each optionally substituted with one or more groups represented by R³; or
R¹ and R², along with the nitrogen to which they are attached, form a monocyclic heterocyclyl optionally substituted by one or more groups selected from halogen, hydroxy, (C₁-C₃)alkyl, halo(C₁-C₃)alkyl, hydroxy(C₁-C₃)alkyl, (C₁-C₃)alkoxy, halo(C₁-C₃)alkoxy, —OC(O)R⁴, —C(O)R⁴, —C(O)OR⁴, —OC(═O)N(R⁴)₂, and oxo; and
each R³ is independently selected from halogen, nitro, cyano, hydroxy, (C₁-C₃)alkyl, halo(C₁-C₃)alkyl, hydroxy(C₁-C₃)alkyl, (C₁-C₃)alkoxy, halo(C₁-C₃)alkoxy, —(CH₂)_{y}—N(R⁴)₂, —(CH₂)_{y}—NR⁴CON(R⁴)₂, —(CH₂)_{y}—CON(R⁴)₂, —(CH₂)_{y}—N(R⁴)COR⁴, —(CH₂)_{y}—CO₂R⁴, —(CH₂)_{y}—OC(O)R⁴, —(CH₂)_{y}—SO₂N(R⁴)₂, —(CH₂)_{y}—SO₂R⁵, —(CH₂)_{y}—NR⁴CO₂R⁴, —(CH₂)_{y}—NR⁴SO₂R⁵ and —(CH₂)_{y}—OC(═O)N(R⁴)₂;
each R⁴ is independently selected from hydrogen and (C₁-C₅)alkyl optionally substituted with halogen, hydroxy or (C₁-C₃)alkoxy;
each R⁵ is independently selected from hydrogen, (C₁-C₅)alkyl and (C₁-C₅)alkoxy, wherein the alkyl is optionally substituted with halogen, hydroxy or (C₁-C₃)alkoxy;
R⁶ is hydrogen, methyl, or ethyl; and
y is 0, 1, 2, or 3;
or a pharmaceutically acceptable salt thereof.

2. The compound of claim 1, wherein the compound is represented by the following structural formula: 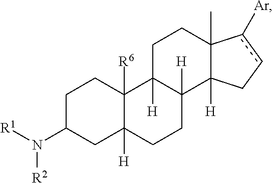
wherein R⁶ is H or methyl, or a pharmaceutically acceptable salt thereof.

3. The compound of claim 2, wherein the compound is represented by a structural formula selected from: 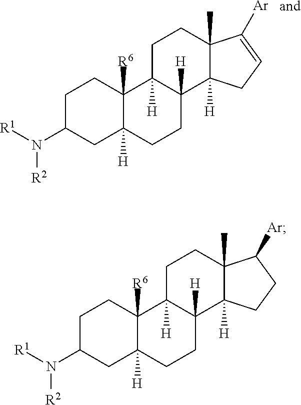
or a pharmaceutically acceptable salt thereof.

4. The compound of claim 3, wherein
R¹ and R² are each independently hydrogen or (C₁-C₁₀)alkyl, each optionally substituted with one or more groups represented by R³; or
R¹ and R², along with the nitrogen to which they are attached, form a monocyclic heterocyclyl optionally substituted by one or more groups selected from halogen, hydroxy, (C₁-C₃)alkyl, halo(C₁-C₃)alkyl, hydroxy(C₁-C₃)alkyl, (C₁-C₃)alkoxy, halo(C₁-C₃)alkoxy, —OC(O)R⁴, —C(O)R⁴, —C(O)OR⁴, —OC(═O)N(R⁴)₂, and oxo; and
Ar is selected from the group consisting of pyrrolidinyl, pyrrolyl, pyrazolyl, triazolyl, tetrazolyl, imidazolyl, piperidinyl, piperazinyl, pyridinyl, pyridazinyl, pyrimidinyl, pyrazinyl, triazinyl, indolyl, isoindolyl, indolinyl, benzoimidazolyl, purinyl, benzotriazolyl, quinolinyl, tetrahydroquinolinyl, isoquinolinyl, or tetrahydroisoquinolinyl, wherein the heterocyclyl or heteroaryl is optionally substituted by one to three groups represented by R³.

5. The compound of claim 4, wherein:
Ar is pyridinyl, quinolinyl, tetrahydroquinolinyl, isoquinolinyl or tetrahydroisoquinolinyl, each optionally substituted by one to three groups represented by R³, or wherein Ar is Ph-(CH₂)ₓN(R⁴)₂;
Ph is phenyl which in addition to (CH₂)ₓN(R⁴)₂ is optionally substituted with one or two groups represented by R³; and
x is an integer from 0 to 3, inclusive.

6. The compound of claim 5, wherein the compound is represented by a structural formula selected from: 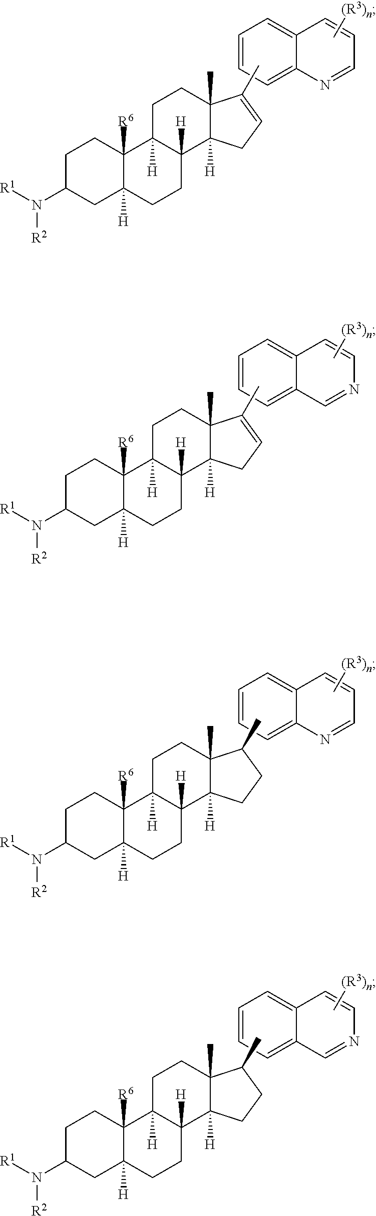 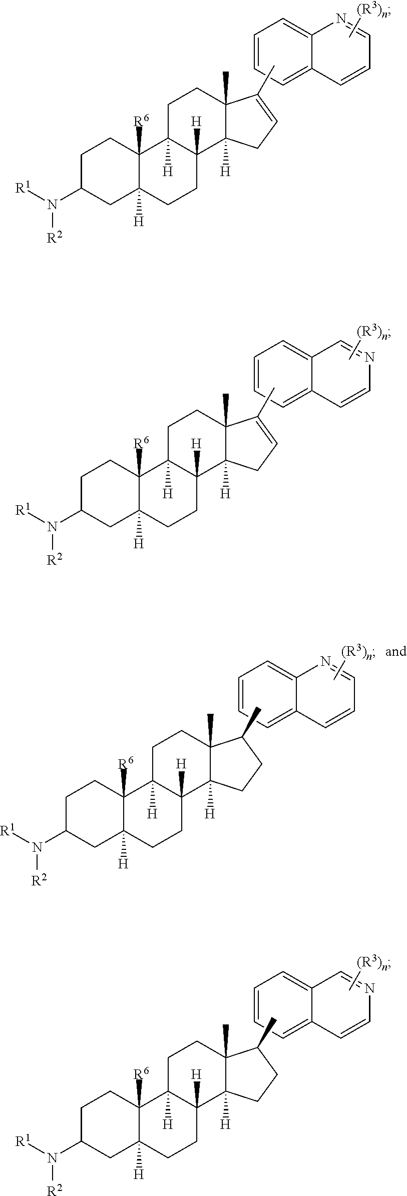
wherein n is an integer from 0 to 3; or a pharmaceutically acceptable salt thereof.

7. The compound of claim 6 wherein:
R¹ and R² are independently hydrogen or (C₁-C₃)alkyl, hydroxy(C₁-C₃)alkyl or (C₁-C₃)alkoxy(C₁-C₃)alkyl; and
each R³ is independently (C₁-C₃)alkyl, hydroxy, (C₁-C₃)alkoxy, halo(C₁-C₃)alkyl, halo(C₁-C₃)alkoxy or hydroxy(C₁-C₃)alkyl.

8. The compound of claim 7 wherein the compound is represented by a structural formula selected from: 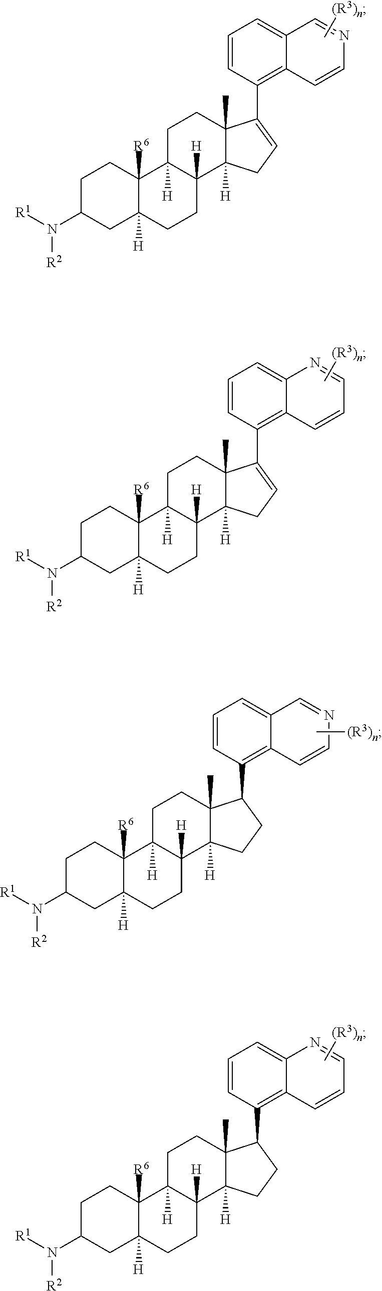 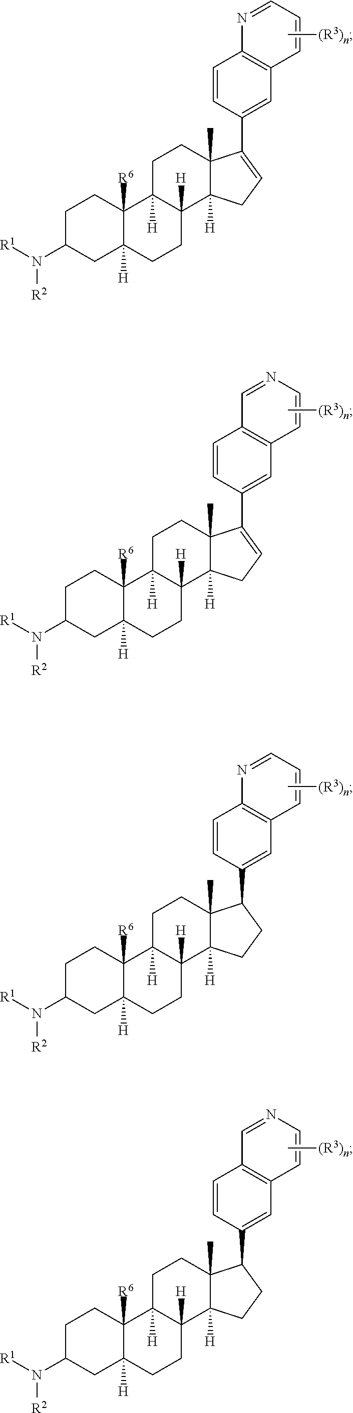 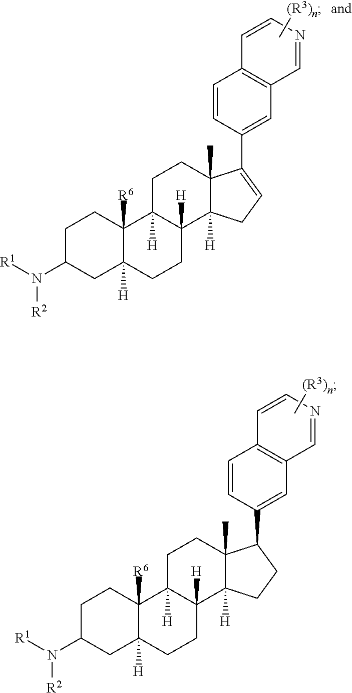
or a pharmaceutically acceptable salt thereof.

9. The compound of claim 1 wherein R¹ and R² are methyl; and n is 0 or 1.

10. The compound of claim 5 wherein the compound is represented by a structural formula selected from: 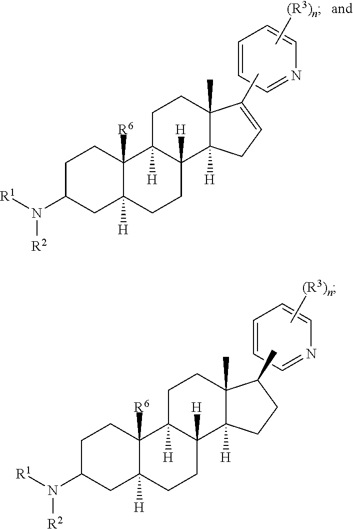
or a pharmaceutically acceptable salt thereof.

11. The compound of claim 10, wherein:
R¹ and R² are independently hydrogen or (C₁-C₃)alkyl, hydroxy(C₁-C₃)alkyl or (C₁-C₃)alkoxy(C₁-C₃)alkyl; and
each R³ is independently (C₁-C₃)alkyl, hydroxy, (C₁-C₃)alkoxy, halo(C₁-C₃)alkyl, halo(C₁-C₃)alkoxy or hydroxy(C₁-C₃)alkyl.

12. The compound of claim 11, wherein the compound is selected from a structural formula selected from: 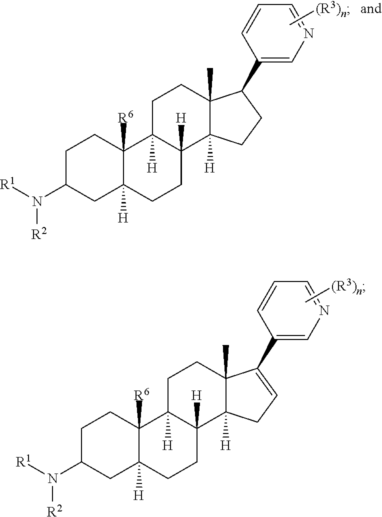
or a pharmaceutically acceptable salt thereof.

13. The compound of claim 12 wherein R¹ and R² are methyl; and n is 0 or 1.

14. The compound of claim 5 wherein the compound is selected from a structural formula selected from: 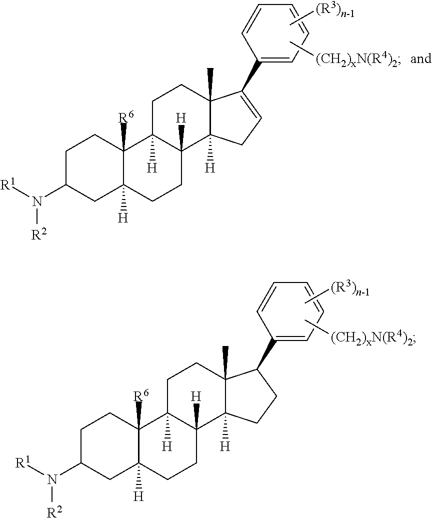
or a pharmaceutically acceptable salt thereof, wherein (CH₂)_{X}N(R⁴)₂ is meta or para to the ring carbon atom that is bonded to the cyclopentane or cyclopentene ring; and n is 1, 2, or 3.

15. The compound of claim 14, wherein:
R¹ and R² are independently hydrogen or (C₁-C₃)alkyl, hydroxy(C₁-C₃)alkyl or (C₁-C₃)alkoxy(C₁-C₃)alkyl;
each R³ is independently (C₁-C₃)alkyl, hydroxy, (C₁-C₃)alkoxy, halo(C₁-C₃)alkyl, halo(C₁-C₃)alkoxy or hydroxy(C₁-C₃)alkyl.

16. The compound of claim 15 wherein R¹ and R² are each methyl; each R⁴ is independently hydrogen or (C₁-C₃)alkyl; x is 1; and n is 1 or 2.

17. The compound of claim 1 wherein:
R¹ and R² are independently hydrogen, (C₁-C₃)alkyl, hydroxy(C₁-C₃)alkyl or (C₁-C₃)alkoxy(C₁-C₃)alkyl; and
each R³ is independently (C₁-C₃)alkyl, hydroxy, (C₁-C₃)alkoxy, halo(C₁-C₃)alkyl, halo(C₁-C₃)alkoxy, or hydroxy(C₁-C₃)alkyl.

18. The compound of claim 17 wherein: R¹ and R² are each methyl; and n is 0 or 1.

19. The compound of claim 1, wherein the compound is represented by the following structural formula: 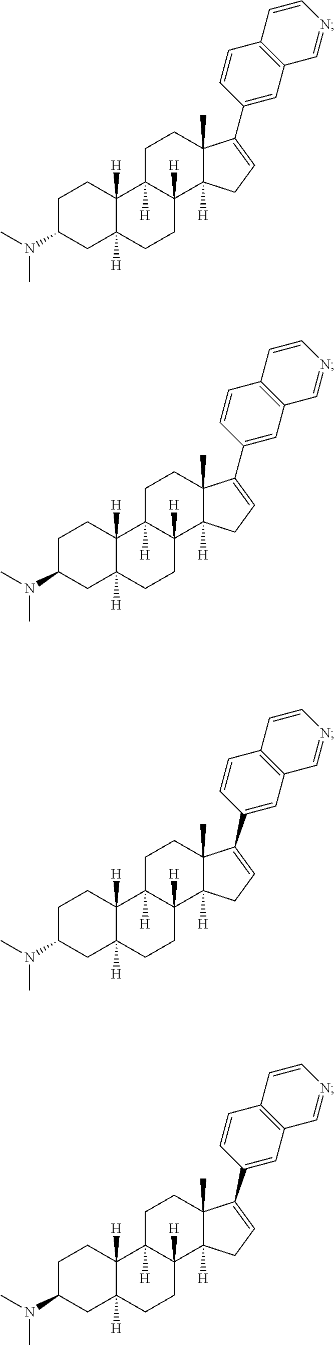 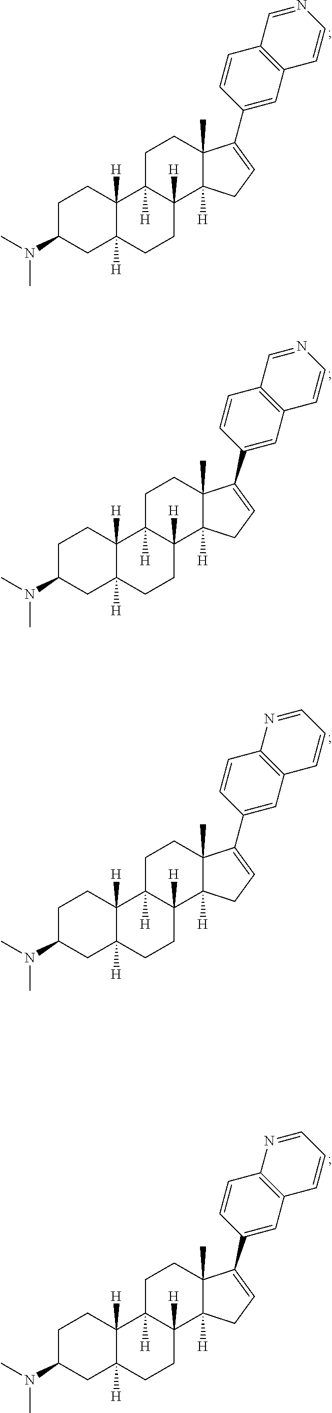 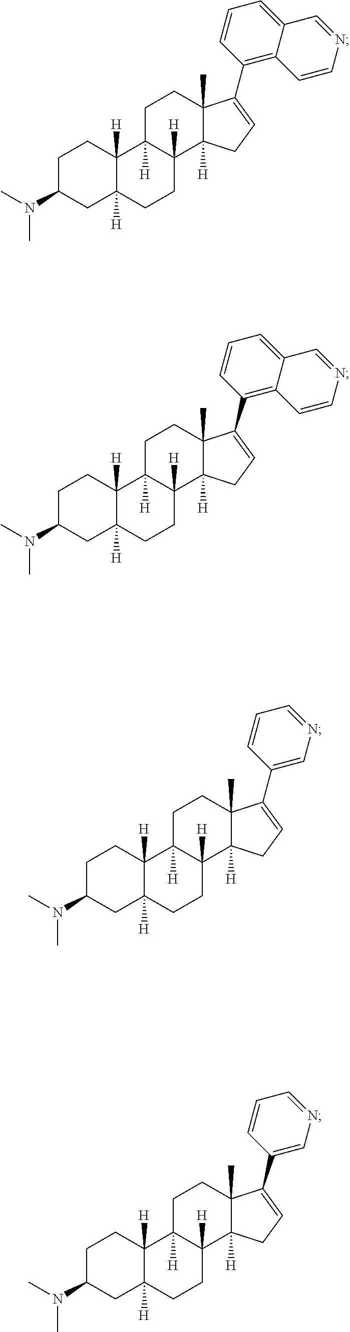 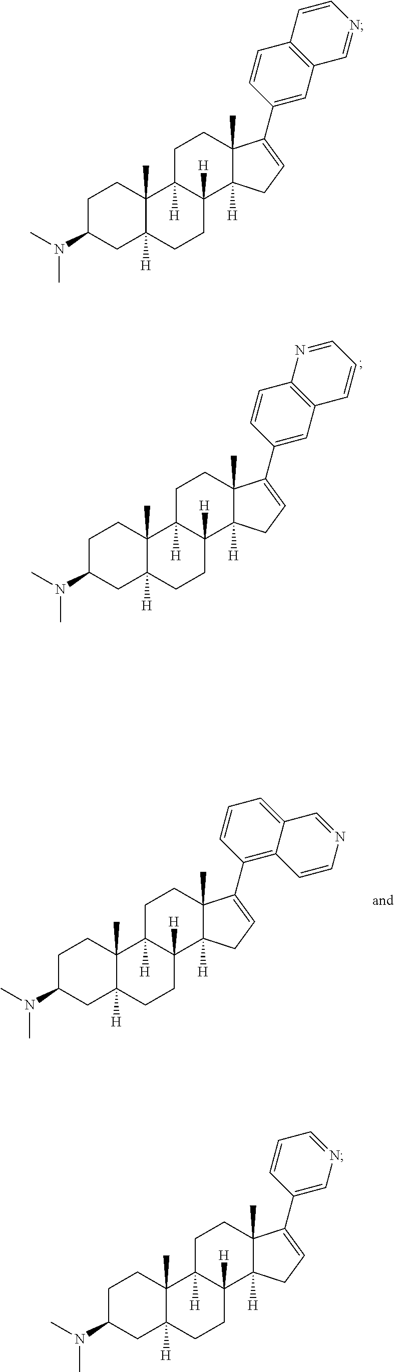
or a pharmaceutically acceptable salt thereof.

20. A pharmaceutical composition comprising: i) a pharmaceutically acceptable carrier or diluent; and ii) a compound of claim 1.

21. A method of inhibiting angiogenesis in a mammalian subject in need thereof, comprising administering to the subject an effective amount of a compound of claim 1.

22-24. (canceled)

25. A method of treating an angiogenesis-related disease or disorder in a mammalian subject, comprising administering to the subject an effective amount of a compound of claim 1.

26. A method of treating macular degeneration in a mammalian subject, comprising administering to the subject an effective amount of a compound of claim 1.
